(19) United States
(12) Patent Application Publication
(10) Pub. No.: US 2012/0196884 A1
(43) Pub. Date: Aug. 2, 2012

(54) UREA SUBSTITUTED SULPHONAMIDE DERIVATIVES

(75) Inventors: Jarkko Tapani Koivunen, Oulu (FI); Jani Korhonen, Turku (FI); Anne Marjamäki, Littoinen (FI); Liisa Nissinen, Turku (FI); Marjo Pihlavisto, Kaarina (FI); Olli Taneli Pentikäinen, Vaajakoski (FI)

(73) Assignee: BIOTIE THERAPIES CORPORATION, Turku (FI)

(21) Appl. No.: 13/378,503

(22) PCT Filed: Jun. 14, 2010
(86) PCT No.: PCT/FI10/50495

§ 371 Date: Feb. 15, 2012

(30) Foreign Application Priority Data
Jun. 16, 2009 (FI) 20095678

(51) Int. Cl. A61K 31/18; C07D 209/42; A61K 31/404; C07D 231/18; A61K 31/415; C07D 409/04; A61K 31/4155; C07D 413/04; A61K 31/422; A61K 31/4436; C07D 401/04; A61K 31/4439; C07D 417/04; A61K 31/427; A61K 31/4245; C07D 333/34; A61K 31/381; C07D 233/84; A61K 31/4164; C07D 409/14; C07D 277/36; C07D 319/18; A61K 31/357; C07D 213/71; A61K 31/4418; C07D 213/54; A61K 31/506; C07D 231/12; A61K 31/4535; A61P 7/02; A61P 29/00; A61P 35/00; A61P 9/00; A61P 1/00; A61P 17/06; A61P 19/02; A61P 25/00; A61P 11/06; A61P 37/08; C07C 303/40; C07C 303/38; C07C 311/44; C07C 311/29; A61K 31/277; C07C 311/21

(52) U.S. Cl. 514/274; 564/84; 514/602; 548/491; 514/419; 548/370.1; 514/407; 548/365.7; 548/236; 514/374; 546/280.4; 514/336; 546/276.1; 514/341; 548/204; 514/365; 548/143; 514/364; 549/65; 514/445; 548/323.5; 514/398; 548/364.7; 548/187; 514/369; 549/362; 514/452; 546/291; 514/346; 546/332; 514/357; 544/333; 514/256; 548/249; 514/378; 548/377.1; 546/213; 514/326; 544/316; 564/86; 514/603; 564/89; 564/92; 514/604; 558/417; 514/522

(57) ABSTRACT

The present invention relates to sulphonamide derivatives, whith a urea moiety. The invention also relates to the use of the derivatives as inhibitors of collagen receptor integrins, especially α2β1 integrin inhibitors e.g. in connection with diseases and medical conditions that involve the action of cells and platelets expressing collagen receptors, their use as a medicament, e.g. for the treatment of thrombosis, inflammation, cancer and vascular diseases, pharmaceutical compositions containing them and a process for preparing them. The sulphonamide derivatives have the general formula (I) or (I′).

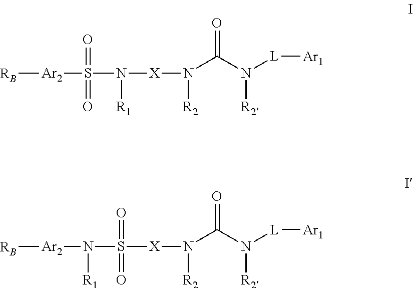

### FIELD OF THE INVENTION

The present invention relates to sulphonamide derivatives, with a urea moiety. The invention also relates to the use of the derivatives as inhibitors of collagen receptor integrins, especially α2β1 integrin inhibitors and more precisely α2β1 integrin I-domain inhibitors, e.g. in connection with diseases and medical conditions that involve the action of cells and platelets expressing collagen receptors, their use as a medicament, e.g. for the treatment of thrombosis, inflammation, cancer and vascular diseases, pharmaceutical compositions containing them and a process for preparing them.

### BACKGROUND OF THE INVENTION

The integrins are a large family of cell adhesion receptors, which mediate anchoring of all human cells to the surrounding extracellular matrix. In addition integrins participate in various other cellular functions, including cell division, differentiation, migration and survival. The human integrin gene family contains 18 alpha integrin genes and 8 beta integrin genes, which encode the corresponding alpha and beta subunits. One alpha and one beta subunit is needed for each functional cell surface receptor. Thus, 24 different alpha-beta combinations exist in human cells. Nine of the alpha subunits contain a specific “inserted” I-domain, which is responsible for ligand recognition and binding. Four of the α I-domain containing integrin subunits, namely α1, α2, α10 and α11, are the main cellular receptors of collagens. Each one of these four alpha subunits forms a heterodimer with beta1 subunit. Thus the collagen receptor integrins are α1β1, α2β1, α10β1 and α11β1 (Reviewed in White et al., Int J Biochem Cell Biol, 2004, 36:1405-1410). Collagens are the largest family of extracellular matrix proteins, composed of at least 27 different collagen subtypes (collagens I-XXVII).

Integrin α2β1 is expressed on epithelial cells, platelets, inflammatory cells and many mesenchymal cells, including endothelial cell, fibroblasts, osteoblasts and chondroblasts (Reviewed in White et al., supra). Epidemiological evidence connect high expression levels of α2β1 on platelets to increased risk of myocardial infarction and cerebrovascular stroke (Santoso et al., Blood, 1999, Carlsson et al., Blood. 1999, 93:3583-3586), diabetic retinopathy (Matsubara et al., Blood. 2000, 95:1560-1564) and retinal vein occlusion (Dodson et al., Eye. 2003, 17:772-777). Evidence from animal models supports the proposed role of α2β1 in thrombosis. Integrin α2β1 is also overexpressed in cancers such as invasive prostate cancer, melanoma, gastric cancer and ovary cancer. These observations connect α2β1 integrin to cancer invasion and metastasis. Moreover, cancer-related angiogenesis can be partially inhibited by anti-α2 function blocking antibodies (Senger et al., Proc. Natl. Acad. Sci. U.S.A., 1997, 94:13612-13617). In addition inflammatory cells are partially dependent on α2β1 function during inflammatory process (de Fougerolles et al., J. Clin. Invest., 2000, 105:721-729; Edelson et al., Blood, 2004, 103:2214-2220). Based on the tissue distribution and experimental evidence α2β1 integrin may be important in inflammation, fibrosis, bone fracture healing and cancer angiogenesis (White et al., supra), while all four collagen receptor integrins may participate in the regulation of bone and cartilage metabolism.

The strong evidence indicating the involvement of collagen receptors in various pathological processes has made them potential targets of drug development. Function blocking antibodies against α1 or α2 subunits have been effective in several animal models including models for inflammatory diseases and cancer angiogenesis. Synthetic peptide inhibitors as well as snake venom peptides blocking the function of α1β1 and α2β1 have been described. (Eble, Curr Pharm Design 2005, 11:867-880). International Patent Publication WO 99/02551 discloses one small molecule drug candidate that regulates the expression of α2β1 but does not actually bind to the integrin.

Publication EP 1 258 252 A1 describes certain N-indolyl-, N-quinolinyl-, N-isoquinolinyl- and N-coumarinyl-arylsulphonamides, which are stated to be integrin expression inhibitors. Said publication does not specifically disclose the compounds of the present invention. Further, said known compounds differ from the compounds now described with respect to their properties and the mechanism of function. The compounds of the present invention are not integrin expression suppressors.

Publication EP 0 472 053 B1 discloses sulphonamides having anti-tumor activity. The compounds specifically described in said publication do not fall within the definition of the compound group of the present invention.

Publication Izvestiya Aakademii Nauk SSSR, Seriya Khimicheskaya (1981), (6), Kravtsov, D. N. et al., pp. 1259-1264 discloses sulphonamides, which are structurally closely related to the compounds now described but which do not fall within the definition of the compound group of the present invention. The field of use of the known compounds is totally different from that of the present invention.

Publication WO 2004/005278 discloses bisarylsulphonamides and their use in cancer therapy.

Publication WO 2007/034035 discloses a group of sulphonamide derivatives, which are useful as inhibitors of collagen receptor integrins.

Also publications WO 00/17159 A1, U.S. Pat. No. 5,939,431 A and U.S. Pat. No. 5,780,483 A disclose amylsulphonamides, which, however, are not described to be useful as inhibitors of collagen receptor integrins.

There is a constant need to develop new compounds, which are potentially useful in treatment of thrombosis, cancer, fibrosis and inflammation through inhibiting collagen receptor integrins. It is essential that collagen receptor integrin inhibitors have high activity, excellent bioactivity and good solubility and/or pharmacological properties.

It has now surprisingly been found that the sulphonamide derivatives with a urea moiety of the present invention are potent inhibitors for collagen receptor integrins, especially α2β1 integrin, and may be used in the treatment of human diseases, such as thrombosis, cancer, fibrosis, inflammation and vascular diseases. The derivatives according to the invention may also be used in diagnostic methods both in vitro and in vivo.

### SUMMARY OF THE INVENTION

The present invention relates to a sulphonamide derivative of formula (I) or (I′) or a physiologically acceptable salt thereof,

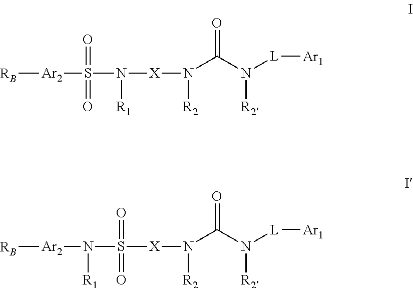

where

R₁ is H, C₁₋₆-alkyl optionally substituted with one or two hydroxyl groups, C₂₋₆-alkenyl optionally substituted with one or two hydroxyl groups, R′R″N—C₁₋₆-alkyl-, C₁₋₆-alkanoyl, R′OOC—C₁₋₆-alkyl-, R′OOC—C₁₋₆-alkoxy- or C₁₋₆-alkoxy-C₁₋₆-alkyl-;

R₂ and R_{2′} are independently selected from H and C₁₋₆-alkyl;

L is absent or a linker, which is a linear or a branched hydrocarbon chain with 1-6 carbon atoms;

X is a 5- or 6-membered aromatic ring with 0-2 heteroatoms selected from N, O and S and optionally substituted with R₃;

R₃ is OH, C₁₋₆-alkyl optionally substituted with one or two hydroxyl groups, C₂₋₆-alkenyl optionally substituted with one or two hydroxyl groups, halo-C₁₋₆-alkyl, halo-C₁₋₆-alkoxy, cyclo-C₃₋₆-alkyl, C₁₋₆-alkoxy, C₁₋₆-alkanoyl, R′OOC—C₁₋₆-alkyl-, R′OOC—C₁₋₆-alkoxy-, —NO₂, —CN, NC—C₁₋₆-alkyl-, halogen, R″R′N—C₁₋₆-alkyl-, R″R′N—C₁₋₆-alkoxy-, R″-C(O)—NR′—C₁₋₆-alkyl-, R″R′N—C(O)—C₁₋₆-alkyl, R″-C(O)—NR′—C₁₋₆-alkoxy-, R″R′N—C(O)—C₁₋₆-alkoxy, —NR′R″, —NR′—C(O)—R″, —C(O)—NHR′, C₁₋₆-alkoxy-C₁₋₆-alkyl- or C₁₋₆-alkoxy-C₁₋₆-alkoxy-;

alternatively R₂ and R₃ form together a moiety selected from one of the following:

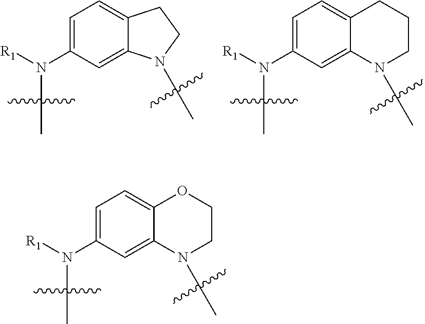

Ar₁ is a 5- or 6-membered saturated or unsaturated ring with 0 to 2 heteroatoms selected independently from N, O and S and optionally substituted with one or more groups selected from C₁₋₆-alkyl optionally substituted with one or two hydroxyl groups, C₂₋₆-alkenyl optionally substituted with one or two hydroxyl groups, halo-C₁₋₆-alkyl, cyclo-C₃₋₆-alkyl, C₁₋₆-alkoxy, halo-C₁₋₆-alkoxy, C₁₋₆-alkanoyl, R′OOC—C₁₋₆-alkyl-, R′OOC—C₁₋₆-alkoxy-, —NO₂, —CN, NC—C₁₋₆-alkyl-, halogen, R″R′N—C₁₋₆-alkyl-, R″R′N—C₁₋₆-alkoxy-, R″-C(O)—NR′—C₁₋₆-alkyl-, R″R′N—C(O)—C₁₋₆-alkyl-, R″-C(O)—NR′—C₁₋₆-alkoxy-, R″R′N—C(O)—C₁₋₆-alkoxy, —NR′R″, —NR′—C(O)—R″, —C(O)—NR″R′, C₁₋₆-alkoxy-C₁₋₆-alkyl- and C₁₋₆-alkoxyC₁₋₆-alkoxy-;

Ar₂ is a ring or a fused ring system, in which the ring or the ring system is unsaturated or saturated, includes 5-12 atoms of which 0-4 are heteroatoms selected from N, O, and S, and is optionally substituted with one or more groups selected from C₁₋₆-alkyl optionally substituted with one or more hydroxyl groups, C₂₋₆-alkenyl optionally substituted with one or two hydroxyl groups, C₁₋₆-alkanoyl, C₁₋₆-alkoxy, C₁₋₆-alkoxy-C₁₋₆-alkyl- and halogen;

R_{B} is a 3-membered hydrocarbon ring or a 4-, 5-, or 6-membered saturated or unsaturated ring with 0 to 3 heteroatoms independently selected from N, O and S and optionally substituted with one or more groups selected from C₁₋₆-alkyl optionally substituted with one or two hydroxyl groups, C₂₋₆-alkenyl optionally substituted with one or two hydroxyl groups, halo-C₁₋₆-alkyl, cyklo-C₃₋₆-alkyl, C₁₋₆-alkoxy, C₁₋₆-alkanoyl, R′OOC—C₁₋₆-alkyl-, R′OOC—C₁₋₆-alkoxy-, R″R′N—C₁₋₆-alkyl-, R″R′N—C₁₋₆-alkoxy-, —NR′R″, pyrrolidyl and halogen;

alternatively R_{B} is selected from H, C₁₋₆-alkyl optionally substituted with one or two hydroxyl groups, C₂₋₆-alkenyl optionally substituted with one or two hydroxyl groups, halo-C₁₋₆-alkyl, halogen, halo-C₁₋₆-alkoxy, —NR′R″, C₁₋₆-alkoxy and —CN;

R′ and R″ are independently selected from H, C₁₋₆-alkyl optionally substituted with one or more hydroxyl groups, C₂₋₆-alkenyl optionally substituted with one or two hydroxyl groups, halo-C₁₋₆-alkyl, C₁₋₆-alkanoyl and C₁₋₆-alkoxyC₁₋₆-alkyl;

provided that

(i) the sulphonamide derivative is not a compound of formula (I) where (a) X is methoxy-substituted phenyl and Ar₂ is pentafluorophenyl, or (b) R₁ is hydrogen and Ar₁ is substituted phenyl; and

(ii) the sulphonamide derivative is not a compound of formula (I′), where L is —CH₂— and Ar₁ is phenyl.

Further the invention relates to derivatives of formula (I) and (I′) for use as inhibitors for collagen receptor integrins specifically α2β1 integrin inhibitors.

The invention also relates to derivatives of formula (I) and (I′) and physiologically acceptable salts thereof for use as a medicament.

Further the invention relates to the use of a derivative of formula (I) and (I′) for preparing a pharmaceutical composition for treating disorders relating to thrombosis, inflammation, cancer and vascular diseases.

The present invention also relates to a pharmaceutical composition comprising an effective amount of a derivative of formula (I) and (I′) or a physiologically acceptable salt thereof and one or more suitable adjuvant.

Further the invention relates to a process for preparing a sulphonamide derivative according to the invention, comprising
reacting a compound of formula (III)

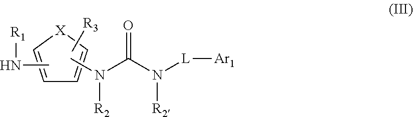

where R₁, R₂, R_{2′}, R₃, X, L, and Ar₁ are as defined in claim 1, with a compound of formula (IV)

R_{B}—Ar₂—SO₂-G (IV)

where R_{B} and Ar₂ is as defined in claim 1 and G is a leaving group, preferably a halogen;

reacting a compound of formula (V)

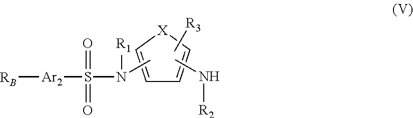

where R₁, R₂, R₃, X, R_{B}, and Ar₂ are as defined in claim 1, with a compound of formula (VI)

G-C(O)NR₂-L-Ar₁ (VI)

where R_{2′}, L and Ar₁ are as defined in claim 1 and G is a leaving group, preferably a halogen; or

reacting a compound of formula (VII)

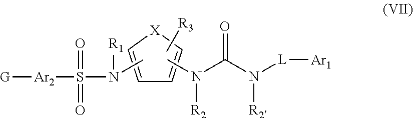

where Ar₂, R₁, R₂, R_{2′}, R₃, X, L and Ar₁ are as defined in claim 1 and G is a leaving group, preferably a halogen, with a compound of formula (VIII)

R_{B}-M (VIII)

where R_{B} is as defined above and M is a leaving group such as a metal.

### FIGURES

FIGS. 1A and B demonstrate that the compound B1 inhibits the platelet adhesion to collagen coated capillary under mimicking physiological flow conditions dose dependently in Cellix analysis.

FIG. 2 demonstrates that compound B94 increases the closure time (Ct) of the whole blood in PFA-100 analysis.

FIG. 3 demonstrates that compound B6 decreases the release of cytokines like IL-6 as an example after induction with LPS.

### DETAILED DESCRIPTION OF THE INVENTION

The present invention relates to sulphonamide derivatives, with a urea moiety, having the general formula (I) or (I′). In the sulphonamide derivative of the present invention the sulphonamide moiety and the urea moiety is separated with a central aromatic ring as defined in claim 1. The sulphonamide moiety is either attached to the central aryl via the nitrogen atom (formula I) or sulphur atom (formula I′).

The central aromatic ring is preferably phenyl, pyrrolyl, furanyl, thiophenyl, pyridinyl or pyrimidinyl. More preferably the sulphonamide derivatives according to the current invention have the general structure Ia or Ia′

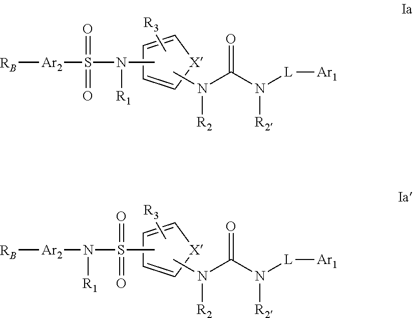

in which x′ is selected from —CH═CH—, —CH═N— and NR′.

In the formulas I and I′ as well as in Ia and Ia′ Ar₁ is preferably an optionally substituted phenyl, Ar₂ is preferably thiophenyl, pyrazolyl or phenyl and further R₁ is preferably H, CH₃, hydroxyethyl or hydroxypropyl. A sulphonamide derivative where R₁ is CH₃, X is —CH═CH—, R₂ and R_{2′} are both H, L is absent and Ar₁ is phenyl is also preferred.

In a preferred embodiment of the present invention the sulphonamide derivative is selected from the group consisting of:

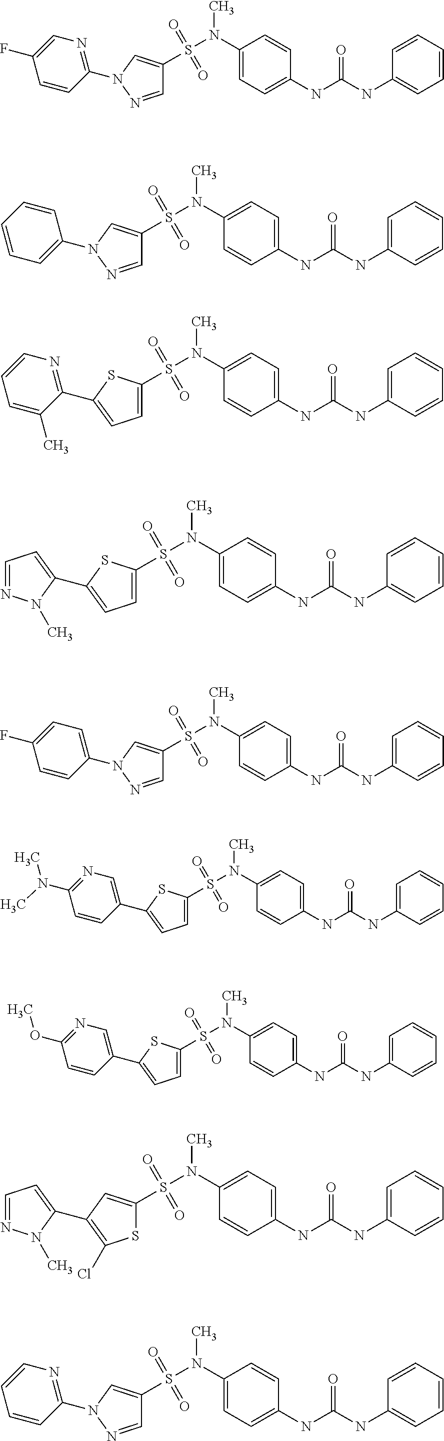

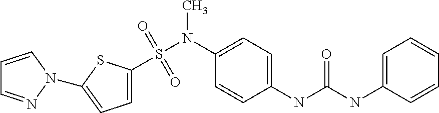

Other typical sulphonamide derivatives of the present invention are presented in table 1.

The term “alkyl” used herein refers to a linear chain alkyl, such as methyl, ethyl, propyl and butyl groups, or branched alkyl group, such as isopropyl and isobutyl groups. Alkyl groups of the current invention typically have from 1 to 6 carbon atoms and preferably 1 to 3 carbon atoms.

The term “alkenyl” used herein refers to a linear or branched hydrocarbon group having at least one carbon-carbon double bond. Alkenyl groups of the current invention typically have from 2 to 6 carbon atoms and preferable 2 to 4 carbon atoms.

The term “alkanoyl” refers to branched or straight chain alkylcarbonyl groups, i.e. alkyl groups with a C═O group, having typically from 1 to 6 carbon atoms, preferably 1 to 3 carbon atoms.

The term “alkoxy” refers to branched or straight chain alkyloxy groups (—O-alkyl) having typically from 1 to 6 carbon atoms, preferably 1 to 3 carbon atoms, in the alkyl moiety.

The term “halo” or “halogen” refers to the non-metal elements of group 17 (IUPAC Style) and is selected from F, Cl, Br and I.

The term “saturated ring” refers to a cyclic hydrocarbon ring or a heterocyclyl with only divalent carbon atoms, i.e. a CH₂ or a substituted CH₂ group, in the ring.

The term “unsaturated ring” refers to aromatic rings or rings with at least one double bond, e.g. a carbon-carbon double bond or a carbon-nitrogen double bond.

The term “fused ring system” refers to a moiety where two or more hydrocarbon rings or heterocycles are fused together, e.g.

The term “linker” refers to a hydrocarbon chain linking two parts of a molecule together, the hydrocarbon chain typically contain 1 to 6 carbon atoms and can be linear, such as —CH₂— and —CH₂CH₂—, or branched such as —CH₂CH(CH₃)CH₂— and CH₂CH(CH₂CH3)CH₂CH₂—.

The term “cycloalkyl” refers to a cyclic hydrocarbon group having typically from 3 to 6 carbon atom, e.g. cyclopropyl and cyclobutyl.

Typical physiologically acceptable salts are e.g. acid addition salts (e.g. HCl, HBr, mesylate, etc.) and alkalimetal and alkaline earth metal salts (Na, K, Ca, Mg, etc.) conventionally used in the pharmaceutical field. Other suitable salts are e.g. ammonium, glucamine, amino acid etc. salts.

The pharmaceutical compositions can contain one or more of the sulphonamides of the invention. The administration can be parenteral, subcutaneous, intravenous, intraarticular, intrathecal, intramuscular, intraperitoneal or intradermal injections, or intravenous infusion, or by transdermal, rectal, buccal, oromucosal, nasal, ocular routes or via inhalation or via implant. Alternatively or concurrently, administration can be by the oral route. The required dosage will depend upon the severity of the condition of the patient, for example, and such criteria as the patient's weight, sex, age, and medical history. The dose can also vary depending upon whether it is to be administered in a veterinary setting to an animal or to a human patient.

For the purposes of parenteral administration, compositions containing the sulphonamides of the invention are preferably dissolved in sterile water for injection and the pH preferably adjusted to about 6 to 8 and the solution is preferably adjusted to be isotonic. If the sulphonamide is to be provided in a lyophilized form, lactose or mannitol can be added as a bulking agent and, if necessary, buffers, salts, cryoprotectants and stabilizers can also be added to the composition to facilitate the lyophilization process, the solution is then filtered, introduced into vials and lyophilized.

Useful excipients for the compositions of the invention for parenteral administration also include sterile aqueous and non-aqueous solvents. The compounds of the invention may also be administered parenterally by using suspensions and emulsions as pharmaceutical forms. Examples of useful non-aqueous solvents include propylene glycol, polyethylene glycol, vegetable oil, fish oil, and injectable organic esters. Examples of aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Examples of solubilizers and co-solvents to improve the aqueous properties of the active compounds to form aqueous solutions to form parenteral pharmaceutical dosage forms are propylene glycol, polyethylene glycols and cyclodextrins. Examples of intravenous infusion vehicles include fluid and nutrient replenishers, electrolyte replenishers, such as those based upon Ringer's dextrose and the like.

Injectable preparations, such as solutions, suspensions or emulsions, may be formulated according to known art, using suitable dispersing or wetting agents and suspending agents, as needed. When the active compounds are in water-soluble form, for example, in the form of water soluble salts, the sterile injectable preparation may employ a non-toxic parenterally acceptable diluent or solvent as, for example, water for injection (USP). Among the other acceptable vehicles and solvents that may be employed are 5% dextrose solution, Ringer's solution and isotonic sodium chloride solution (as described in the Ph. Eur/USP). When the active compounds are in a non-water soluble form, sterile, appropriate lipophilic solvents or vehicles, such as fatty oil, for example, sesame oil, or synthetic fatty acid esters, for example, ethyl oleate or triglycerides, are used. Alternatively, aqueous injection suspensions which contain substances which increase the viscosity, for example, sodium carboxymethyl cellulose, sorbitol, and/or dextran, and optionally also contain stabilizers may be used.

Pharmaceutical preparations for oral (but systemic) administration can be obtained by combining the active compounds with solid excipients, optionally granulating a resulting mixture and processing the mixture or granules or solid mixture without granulating, after adding suitable auxiliaries, if desired or necessary, to give tablets or capsules after filling into hard capsules.

Suitable excipients are, in particular, fillers such as sugars, for example lactose or sucrose, mannitol or sorbitol, cellulose and/or starch preparations and/or calcium phosphates, for example tricalcium phosphate or calcium hydrogen phosphate, as well as binders, such as starches and their derivatives, pastes, using, for example, maize starch, wheat starch, rice starch, or potato starch, gelatine, tragacanth, methyl cellulose, hydroxypropylmethyl cellulose, sodium carboxymethyl cellulose, and/or polyvinyl pyrrolidone, derivatives, and/or, if desired, disintegrating agents, such as the above-mentioned starches, and also carboxymethyl-starch, cross-linked polyvinyl pyrrolidone, agar or alginic acid or a salt thereof, such as sodium alginate. Auxiliaries are, above all, flow-regulating agents and lubricants, for example, silica, talc, stearic acid or salts thereof, such as magnesium stearate or calcium stearate, with suitable coating, which if desired, are resistant to gastric juices and for this purpose, inter alia concentrated sugar solutions, which optionally contain gum arabic, talc, polyvinyl pyrrolidone, polyethylene glycol and/or titanium dioxide, lacquer solutions and suitable organic solvents or solvent mixtures, but also film coating using e.g. cellulose derivatives, polyethylene glycols and/or PVP derivatives may be used. In order to produce coatings resistant to gastric juices, solutions of suitable cellulose preparations such as acetyl cellulose phthalate or hydroxypropylmethyl cellulose phthalate, are used for coating. Dyestuffs or pigments may be added to the tablets or dragee coatings or to coatings for example, for identification or in order to characterize different combinations of active compound doses.

Solid dosage forms for oral administration include capsules, tablets, pills, troches, lozenges, powders and granules. In such solid dosage forms, the active compound may be admixed with at least one inert diluent such as sucrose, lactose or starch. Such dosage forms may also comprise, as is normal practice, pharmaceutical adjuvant substances, e.g., stearate lubricating agents or flavouring agents. Solid oral preparations can also be prepared with enteric or other coatings which modulate release of the active ingredients.

Liquid dosage forms for oral administration include pharmaceutically acceptable emulsions, solutions, suspensions, syrups and elixirs containing inert non-toxic diluents commonly used in the art, such as water and alcohol. Such compositions may also comprise adjuvants, such as wetting agents, buffers, emulsifying, suspending, sweetening and flavouring agents.

The compositions of the invention may also be administered by means of pumps, or in sustained-release form. The compounds of the invention may also be delivered to specific organs in high concentration by means of suitably inserted catheters, or by providing such molecules as a part of a chimeric molecule (or complex) which is designed to target specific organs.

Administration in a sustained-release form is more convenient for the patient when repeated injections for prolonged periods of time are indicated so as to maximize the comfort of the patient. Controlled release preparation can be achieved by the use of polymers to complex or adsorb the cornpounds of the invention. Controlled delivery can be achieved by selecting appropriate macromolecules (for example, polyesters, polyamino acids, polyvinyl pyrrolidone, ethylenevinylacetate, methylcellulose, carboxymethylcelluloase protamine zinc and protamine sulfate) as well as the method of incorporation in order to control release. Another possible method to control the duration of action by controlled release preparations is to incorporate the desired compounds into particles of a polymeric material such as polyesters, polyamino acids, hydrogels, poly (lactic acid) or ethylene vinylacetate copolymers. Alternatively, instead of incorporating the sulphonamide into these polymeric particles, the sulphonamide can be entrapped into microparticles, prepared, for example, by coacervation techniques or by interfacial polymerization, for example, hydroxymethylcellulose or gelatin-microcapsules and poly (methylmethacrylate) microcapsules, respectively, or in colloidal drug delivery systems, for example liposomes, albumin microspheres, microemulsions, nanoparticles, and nanocapsules or in macroemulsions. The above-mentioned technique may be applied to both parenteral and oral administration of the pharmaceutical formulation.

The pharmaceutical compositions of the present invention can be manufactured in a manner which is in itself known, for example, by means of conventional mixing, granulating, dragee-making, dissolving, lyophilizing or similar processes.

The compounds of the invention are potent collagen receptor inhibitors and useful for inhibiting or preventing the adhesion of cells on collagen or the migration and invasion of cells through collagen containing matrices, in vivo or in vitro. The now described compounds inhibit the migration of malignant cells and are thus for treating diseases such as cancers, including prostate, and melanoma, especially where α2β1 integrin dependent cell adhesion/invasion/migration may contribute to the malignant mechanism.

The compounds of the invention also inhibit inflammatory responses that require α2β1 integrin. The now described compounds can inhibit pathological inflammatory processes and can thus be used for treating diseases like inflammatory bowel disease, psoriasis, arthritis, multiple sclerosis, asthma, and allergy.

The compounds of the invention also inhibit adhesion of platelets to collagen and collagen-induced platelet aggregation. Thus, the compounds of the invention are useful for treating patients in need of preventative or ameliorative treatment for thromboembolic conditions i.e. diseases that are characterized by a need to prevent adhesion of platelets to collagen and collagen-induced platelet aggregation, for example treatment and prevention of stroke, myocardial infraction unstable angina pectoris diabetic rethinopathy or retinal vein occlusion.

### Pharmacological Tests

### Adhesion Assay Method

Chinese Hamster Ovary (CHO) cell clone expressing wild type α2 integrin was used in cell adhesion assay. Cells were suspended in serum free medium containing 0.1 mg/mL cycloheximide (Sigma) and the compounds were preincubated with the cells prior to transfer to the wells. Cells (150000/well) were allowed to attach on collagen type I coated wells (in the presence and absence of inhibitor compounds) for 2 h at +37° C. and after that non-adherent cells were removed. Fresh serum free medium was added and the living cells were detected using a cell viability kit (Roche) according to the manufacturer's protocol.

The following examples illustrate the invention but are not intended to limitate the scope of the invention (Table 1).

TABLE 1
CHO-α2β1 cell adhesion to collagen type I in the precence of inhibitory
compounds. Determined EC50 values represent the concentratiosn
required for half maximal inhibitory effect.

| | Adh EC50 | |
|---|---|---|
| Compound | (μM) | Structure |
| B1 | 0.064 | 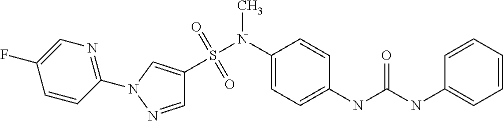 |
| B2 | 0.17 | 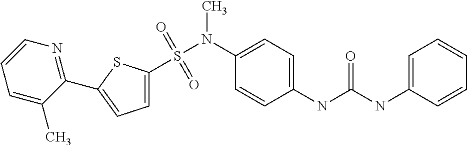 |
| B3 | 0.13 | 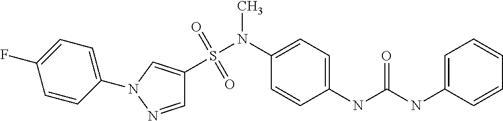 |
| B4 | 0.14 | 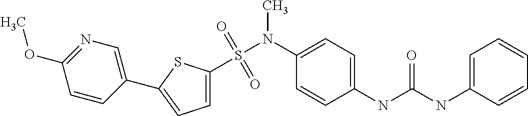 |
| B5 | 0.14 | 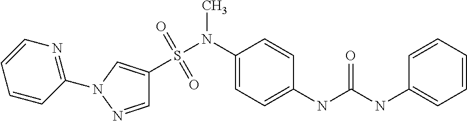 |
| B6 | 0.16 | 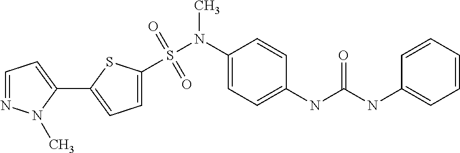 |
| B7 | 0.11 | 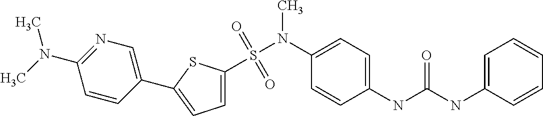 |
| B8 | 0.19 | 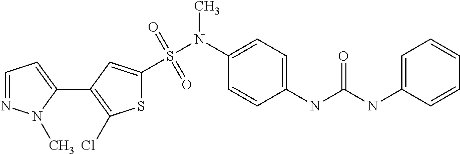 |
| B9 | 0.21 | 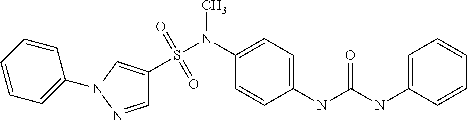 |
| B10 | 0.22 | 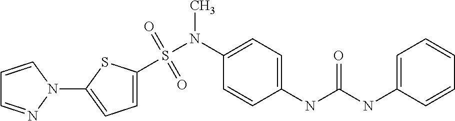 |
| B11 | 0.12 | 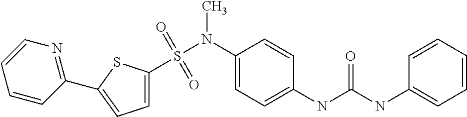 |
| B12 | 0.26 | 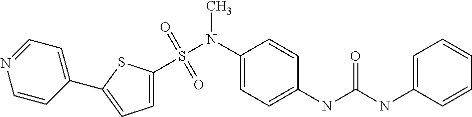 |
| B13 | 0.26 | 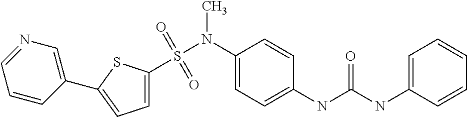 |
| B14 | 0.37 | 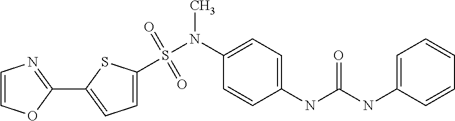 |
| B15 | 0.31 | 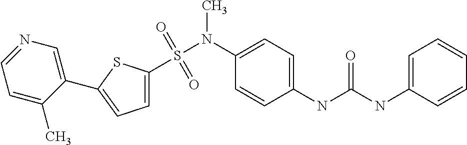 |
| B16 | 0.34 | 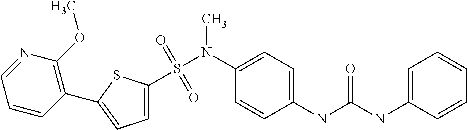 |
| B17 | 0.35 | 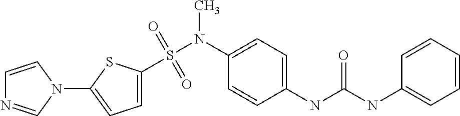 |
| B18 | 0.39 | 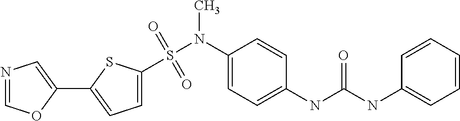 |
| B19 | 0.4 | 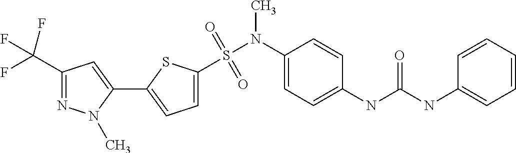 |
| B20 | 0.43 | 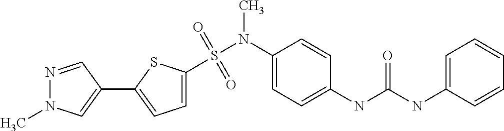 |
| B21 | 0.43 | 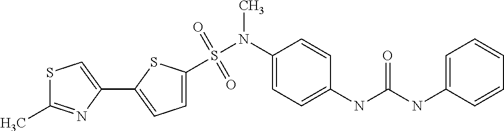 |
| B22 | 0.57 | 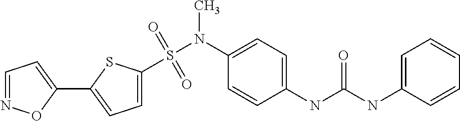 |
| B23 | 0.56 | 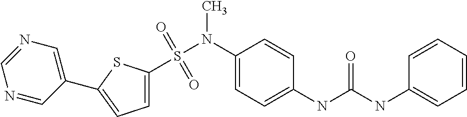 |
| B24 | 0.71 | 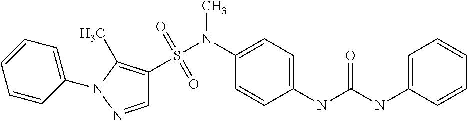 |
| B25 | 0.74 | 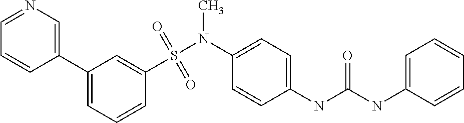 |
| B26 | 0.81 | 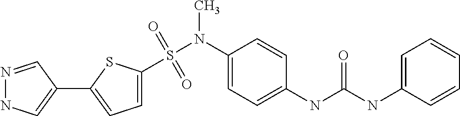 |
| B27 | 0.85 | 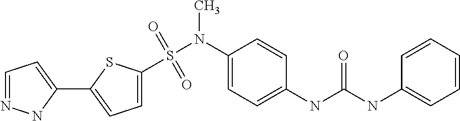 |
| B28 | 0.97 | 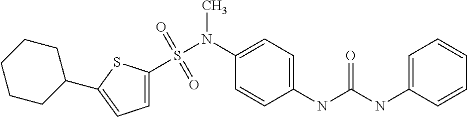 |
| B29 | 1.1 | 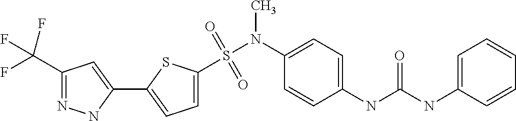 |
| B30 | 1.2 | 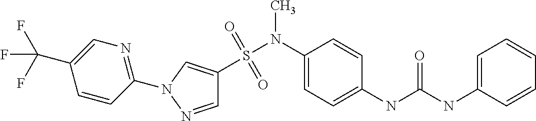 |
| B31 | 1.2 | 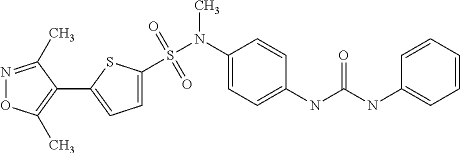 |
| B32 | 1.2 | 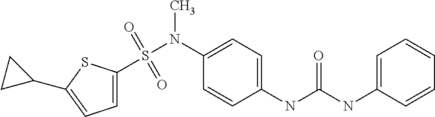 |
| B33 | 1.3 | 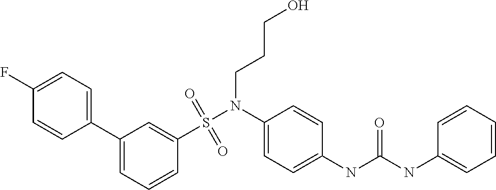 |
| B34 | 1.3 | 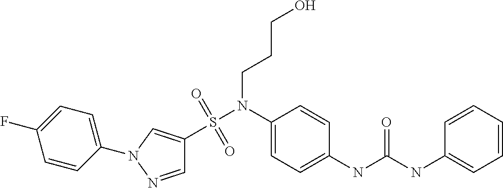 |
| B35 | 1.4 | 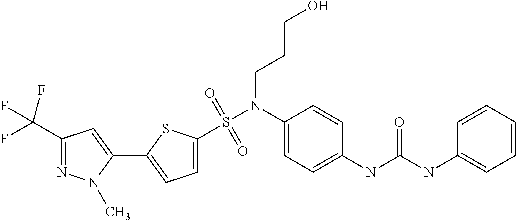 |
| B36 | 1.6 | 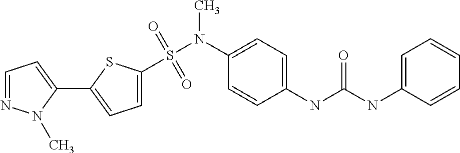 |
| B37 | 1.7 | 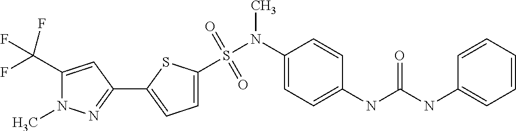 |
| B38 | 1.7 | 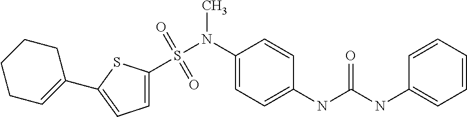 |
| B39 | 1.8 | 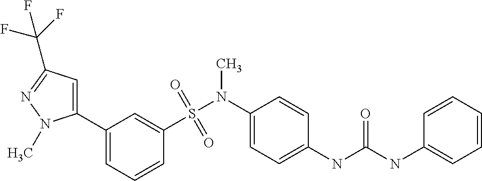 |
| B40 | 1.9 | 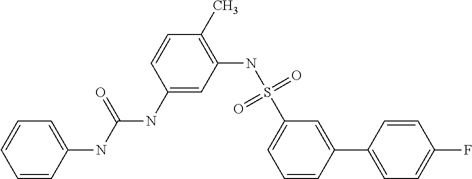 |
| B41 | 1.9 | 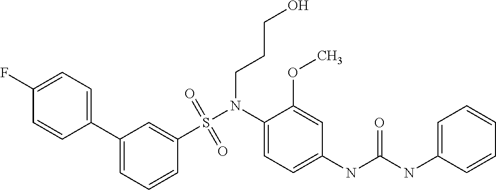 |
| B42 | 2 | 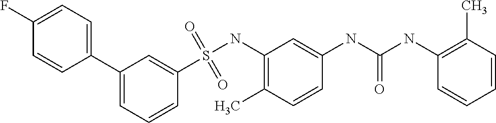 |
| B43 | 2 | 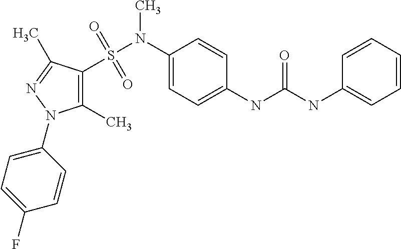 |
| B44 | 2 | 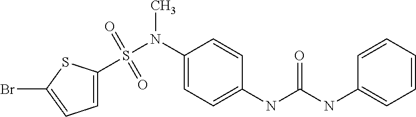 |
| B45 | 2.1 | 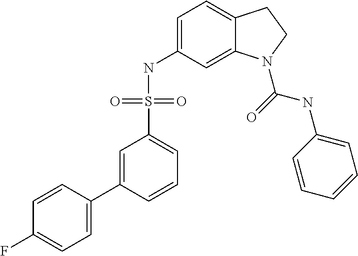 |
| B46 | 2.2 | 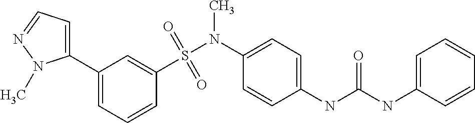 |
| B47 | 2.3 | 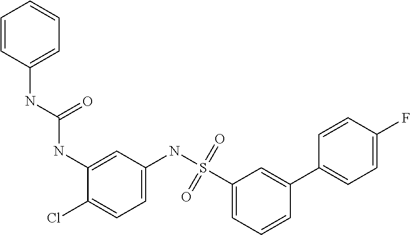 |
| B48 | 2.3 | 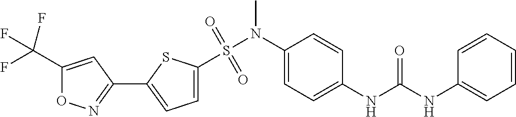 |
| B49 | 2.5 | 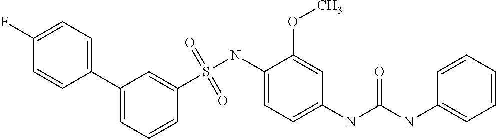 |
| B50 | 2.6 | 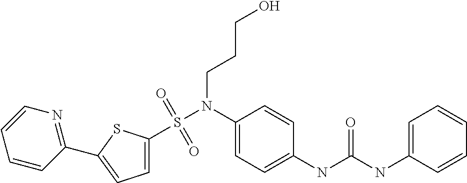 |
| B51 | 2.6 | 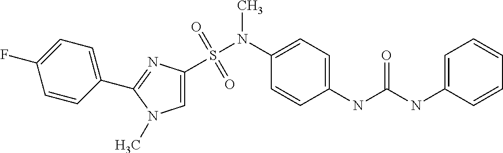 |
| B52 | 3 | 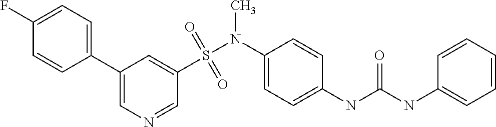 |
| B53 | 3 | 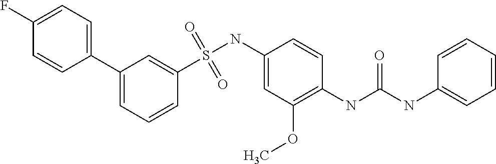 |
| B54 | 3 | 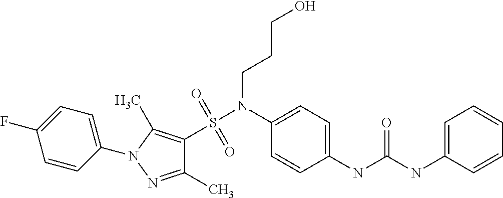 |
| B55 | 3.1 | 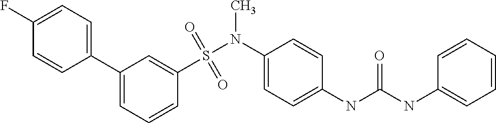 |
| B56 | 3.2 | 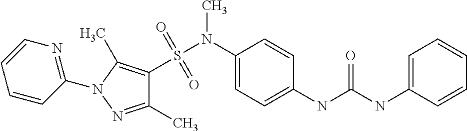 |
| B57 | 3.8 | 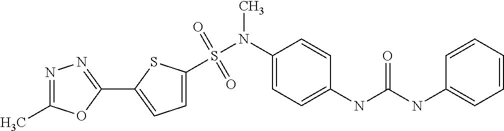 |
| B58 | 3.9 | 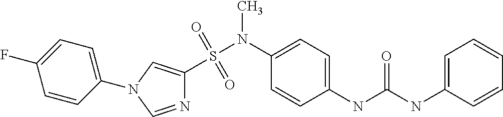 |
| B59 | 4 | 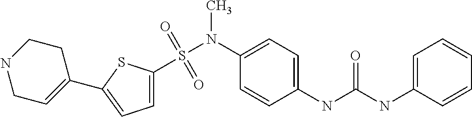 |
| B60 | 4 | 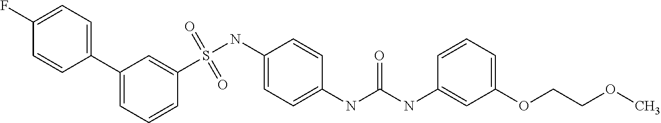 |
| B61 | 4 | 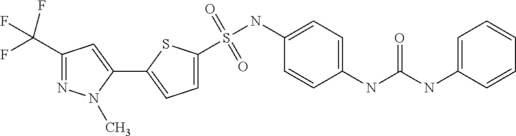 |
| B62 | 4.1 | 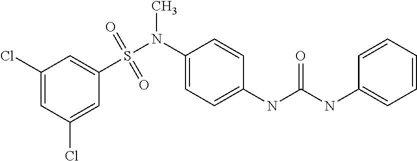 |
| B63 | 4.2 | 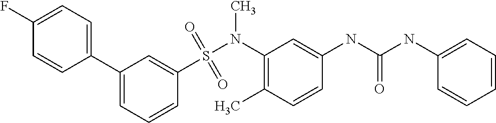 |
| B64 | 4.3 | 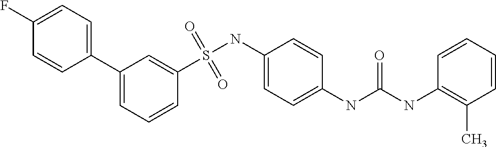 |
| B65 | 4.5 | 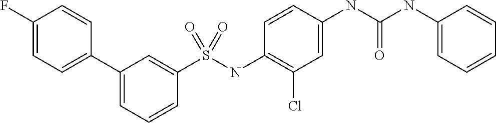 |
| B66 | 4.7 | 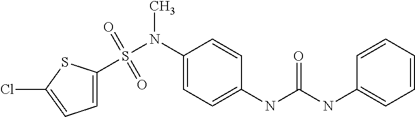 |
| B67 | 6.8 | 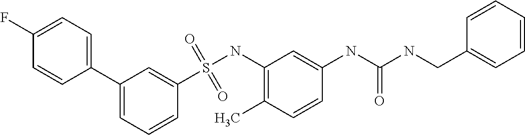 |
| B68 | 5.1 | 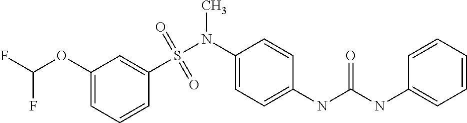 |
| B69 | 5.5 | 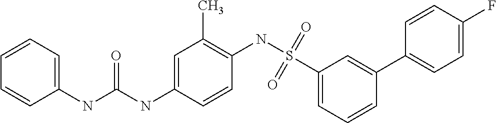 |
| B70 | 5.5 | 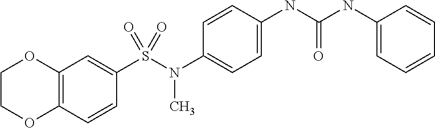 |
| B71 | 5.5 | 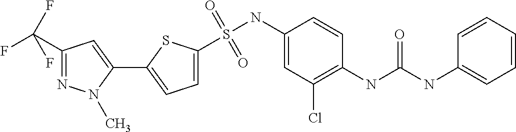 |
| B72 | 5.7 | 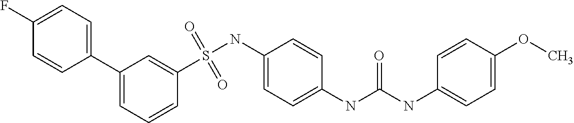 |
| B73 | 5.8 | 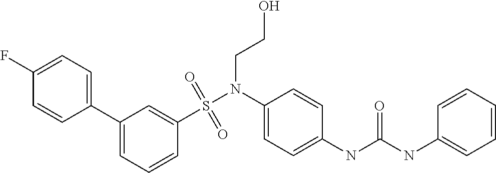 |
| B74 | 5.8 | 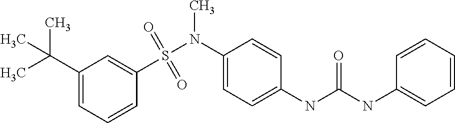 |
| B75 | 6.3 | 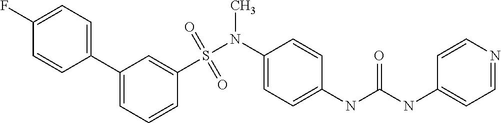 |
| B76 | 6.6 | 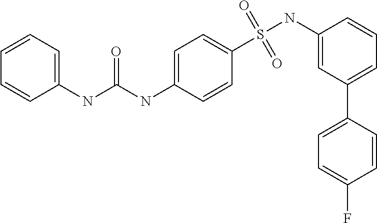 |
| B77 | 6.7 | 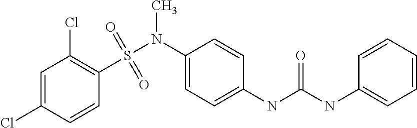 |
| B78 | 6.7 | 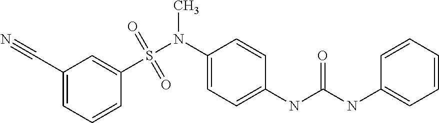 |
| B79 | 7 | 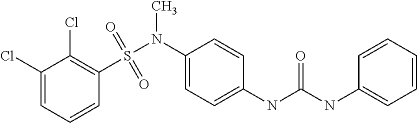 |
| B80 | 7.2 | 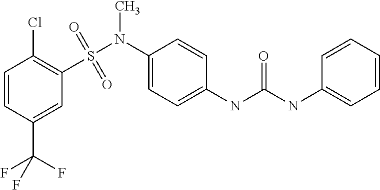 |
| B81 | 7.4 | 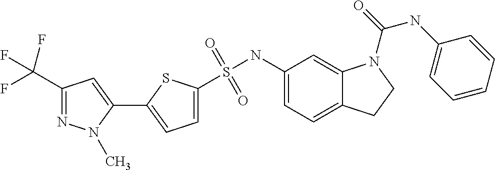 |
| B82 | 7.6 | 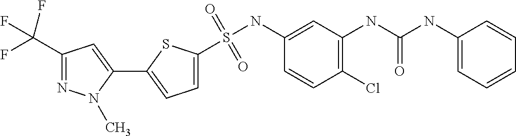 |
| B83 | 7.6 | 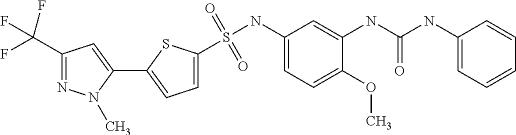 |
| B84 | 7.7 | 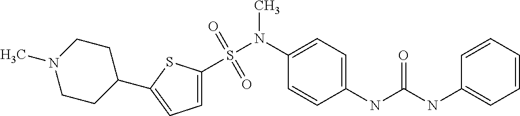 |
| B85 | 7.7 | 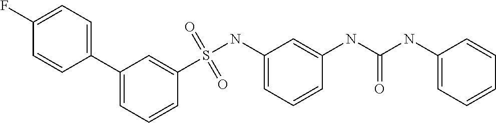 |
| B86 | 7.7 | 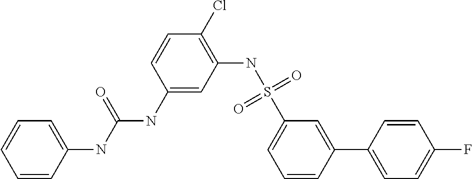 |
| B87 | 7.7 | 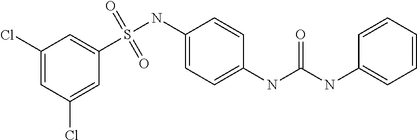 |
| B88 | 7.8 | 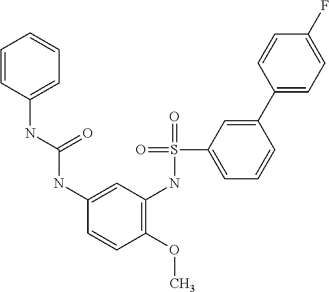 |
| B89 | 8 | 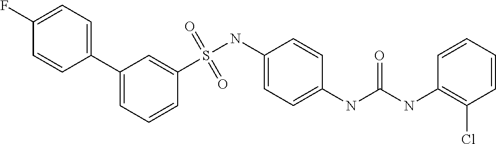 |
| B90 | 8 | 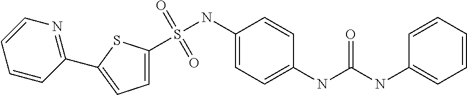 |
| B91 | 8.1 | 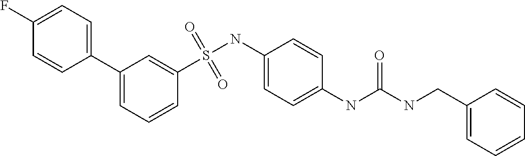 |
| B92 | 8.2 | 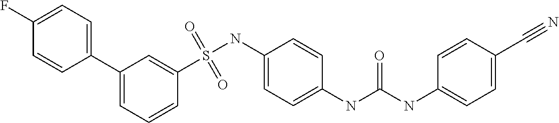 |
| B93 | 9.9 | 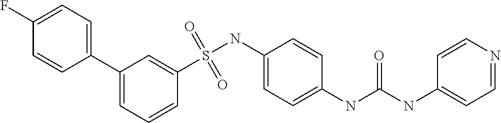 |
| B94 | 10 | 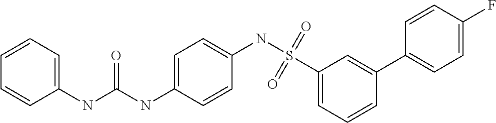 |
| B95 | 10 | 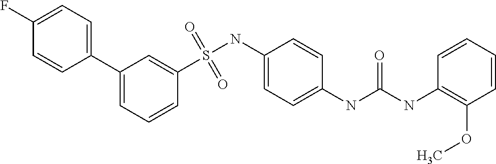 |
| B96 | 10 | 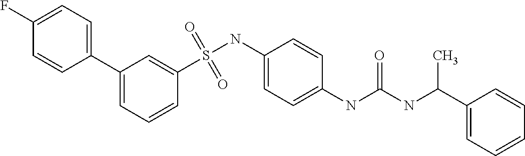 |
| B97 | 10 | 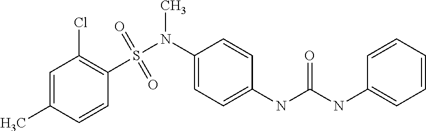 |
| B98 | 11 | 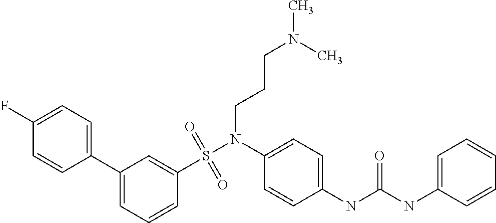 |
| B99 | 11 | 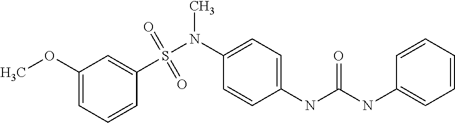 |
| B100 | 13 | 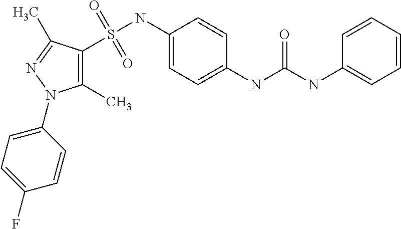 |
| B101 | 14 | 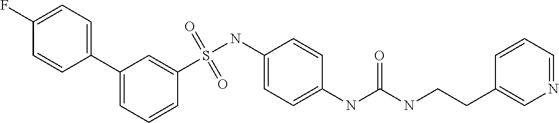 |
| B102 | 16 | 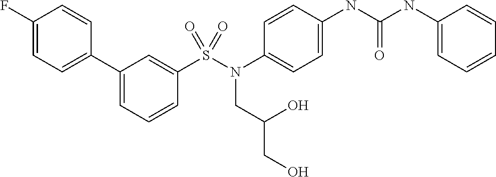 |
| B103 | 17 | 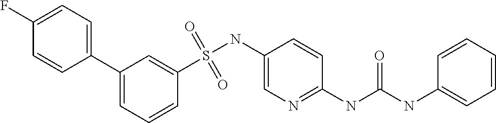 |
| B104 | 17 | 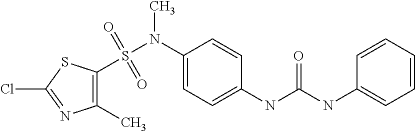 |
| B105 | 0.92 | 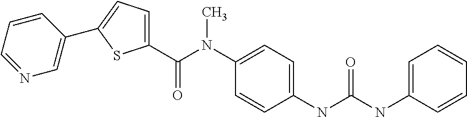 |
| B106 | 2.6 | 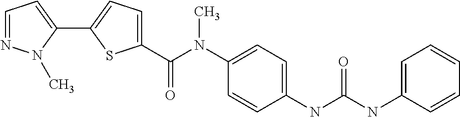 |
| B107 | 0.18 | 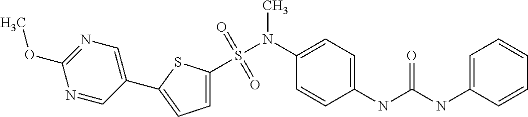 |
| B108 | 4.3 | 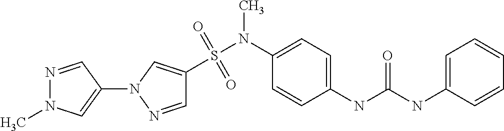 |
| B109 | 0.12 | 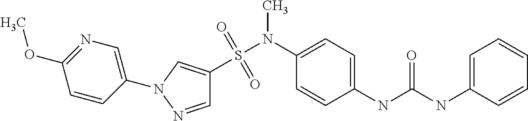 |
| B110 | 0.12 | 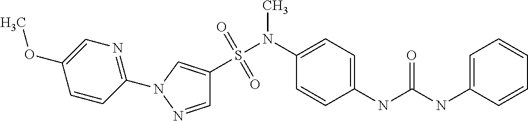 |
| B111 | 1.6 | 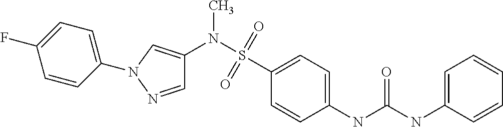 |
| B112 | 3.8 | 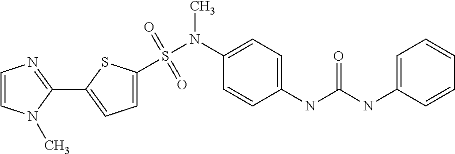 |
| B113 | 11 | 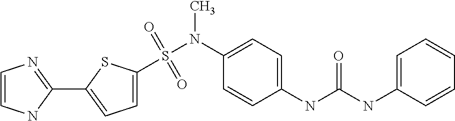 |
| B114 | 4.5 | 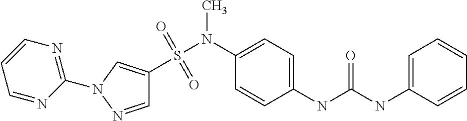 |
| B115 | 5.8 | 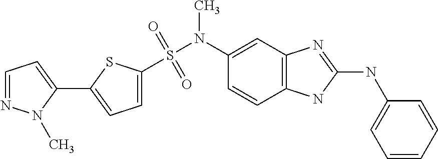 |
| B116 | 0.16 | 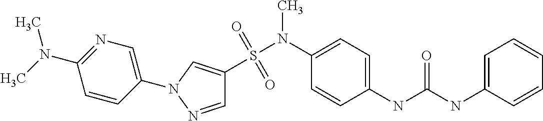 |
| B117 | 0.11 | 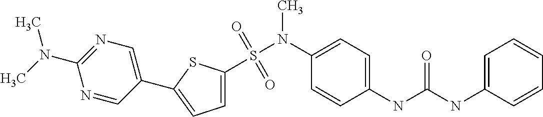 |
| B118 | 0.41 | 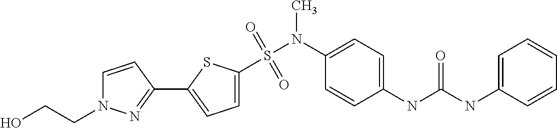 |
| B119 | 2.4 | 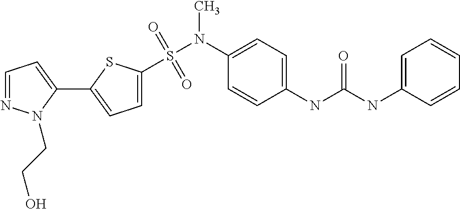 |
| B120 | 1.1 | 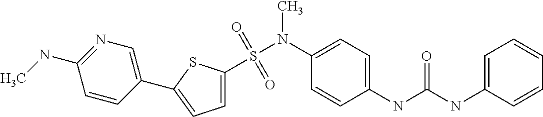 |
| B121 | 0.29 | 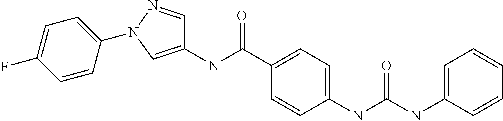 |
| B122 | nd | 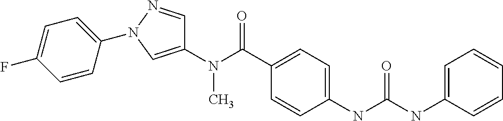 |
| B123 | nd | 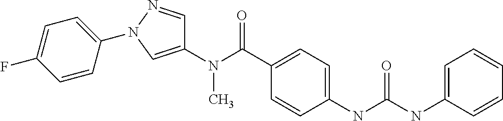 |
| B124 | 0.49 | 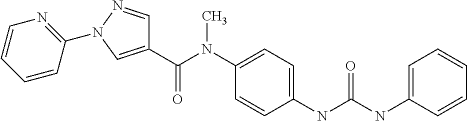 |
| B125 | nd | 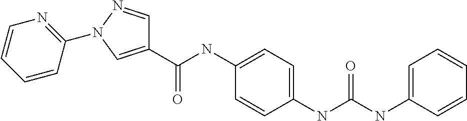 |
| B126 | nd | 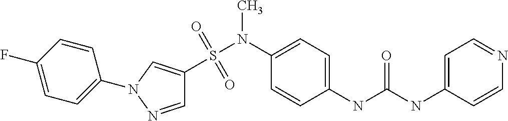 |
| B127 | 6.6 | 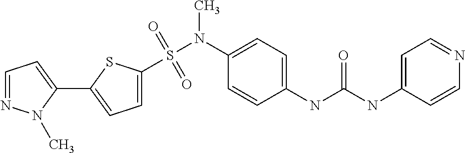 |
| B128 | 1.6 | 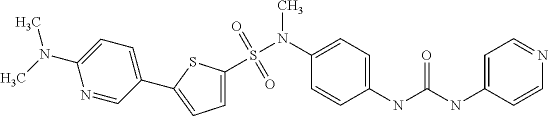 |

### A Cell Invasion Assay was Used to Demonstrate the Anti-Cancer Potential of the Inhibitors In Vitro

The ability to interact with extracellular matrix basement membranes is essential for the malignant cancer cell phenotype and cancer spread. α2β1 levels are known to be upregulated in tumorigenic cells. The overexpression regulates cell adhesion and migration to and invasion through the extracellular matrix. By blocking the interaction between extracellular matrix components like collagen and α2β1 it is possible to block cancer cell migration and invasion in vitro. Prostate cancer cells (PC-3) expressing α2β1 endogenously were used to test the in vitro anticancer potential of the inhibitors of the present invention.

### Experimental Procedure

Invasion of PC-3 cells (CRL-1435, ATCC) through Matrigel was studied using BD Biocoat invasion inserts (BD Biosciences). Inserts were stored at −20° C. Before the experiments inserts were allowed to adjust to the room temperature. 500 μl of serum free media (Ham's F12K medium, 2 mM Lglutamine, 1.5 g/l sodium bicarbonate) was added into the inserts and allowed to rehydrate at 37° C. in cell incubator for two hours. The remaining media was aspirated. PC-3 cells were detached, pelleted and suspended into serum free media (50 000 cells/500 μL). 300 μL of cell suspension was added into the insert in the absence (control) or presence of the inhibitor according to the present invention. Inserts were placed on the 24-well plates; each well containing 700 μL of cell culture media with 3% of fetal bovine serum as chemoattractant. Cells were allowed to invade for 72 hours at 37° C. in cell incubator. Inserts were washed with 700 μL PBS, and fixed with 4% paraformaldehyde for 10 minutes. Paraformaldehyde was aspirated and cells were washed with 700 μL of PBS and inserts were stained by incubation with hematoxylin for 1 minute. The stain was removed by washing the inserts with 700 μL of PBS. Inserts were allowed to dry. Fixed invaded cells were calculated under the microscope. Invasion % was calculated as a comparison to the control.

Cell invasion assay is used as an in vitro cancer metastatis model. The sulfonamide molecules have been shown to inhibit tumor cell invasion in vitro (see table 2). Some structures inhibit invasion even with submicromolar concentrations.

TABLE 2
Analysis of inhibition of PC-3 cell invasion through Matrigel.
Inhibition (%) of invasion at 5 μM is shown.

| | Inh % |
|---|---|
| Compound | at 5 μM |
| B129 | 65 |
| B130 | 100 |
| B131 | 83 |
| B132 | 100 |
| B133 | 70 |
| B134 | 72 |
| B135 | 99 |
| B136 | 3 |
| B137 | 100 |
| B138 | 86 |
| B139 | 16 |
| B140 | 84 |
| B141 | 70 |
| B142 | 95 |
| B143 | 73 |
| B144 | 64 |
| B145 | 27 |
| | (1 uM) |
| B94 | 40 |
| B146 | 76 |
| B147 | 81 |
| B149 | 92 |
| B150 | 68 |
| B160 | 24 |
| B64 | 42 |
| B161 | 45 |
| B162 | 57 |
| B72 | 39 |
| B163 | 82 |
| | (2 uM) |
| B55 | 76 |
| B164 | 95 |
| B91 | 56 |
| B77 | 40 |
| B47 | 96 |
| B40 | 98 |
| B165 | 88 |
| B86 | 44 |
| B67 | 10 |
| B80 | 43 |
| B78 | 30 |
| B68 | 29 |
| B60 | 70 |
| B52 | 25 |
| B166 | 72 |
| B75 | 38 |
| B63 | 45 |

### Cellix Microfluidic System was Used to Demonstrate the Antithrombotic Potential of the α2β1 Inhibitors

Cellix system was used to demonstrate the possible antithrombotic effects of α2β1 modulators in flow conditions. Cellix microfluidic platform models human blood vessels providing a dynamic set-up mimicking physiological conditions to test new therapeutic agents (Cellix Ltd). The platform was used to measure the platelet adhesion to collagen coated capillary under flow. An anti-coaculated whole blood sample was run through a collagen coated capillary under a constant shear and the size of thrombi on capillary wall was analyzed with analysis program (DucoCell, Cellix Ltd). If the average thrombi area was decreased when compared to the control sample the compound was suggested to have antithrombotic activity.

### Experimental Procedure

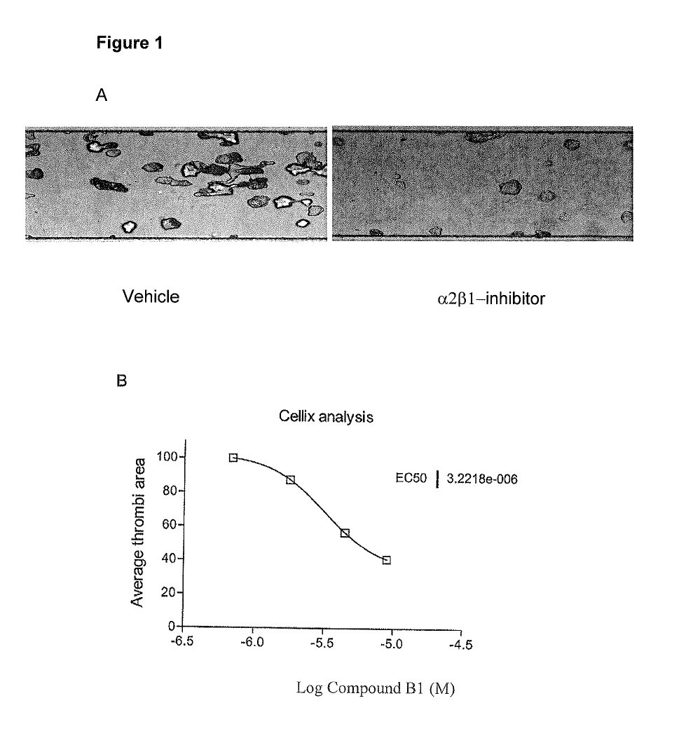

The capillary were coated with Horm collagen 20 μg/mL (Nycomed) and incubated for 24 h in +4° C. Background was blocked with 1% BSA (bovine serum albumin, Sigma) treatment for 30 min in room temperature. Blood was collected from a donor into blood collection tubes containing 40 μM PPACK (Dphenylalanyl-L-prolyl-L-arginine chloromethyl-ketone, Calbiochem) as anticoagulant. Blood was treated either with inhibitory compounds or vehicle controls. Samples were kept at room temperature for 5 minutes. The samples were run through the capillary with the constant shear rate (90 dynes/cm², Mirus 1.0 Nanopump, Cellix Ltd) for 5 min and capillary was washed with JNL buffer (6 mM Dextrose, 0.13 M NaCl, 9 mM Na Bicarb, 10 mM Na Citrate, 10 mM Tris base, 3 mM KCl, 0.81 mM KH₂PO₄, 0.9 mM MgCl₂; pH was adjusted to 7.35 with 19 mM Citrate acid anhydrous, 37 mM Sodium citrate, 67 mM Dextrose) with the constant shear rate (90 dynes/cm²) for 2 min. After that the average thrombi area on the capillary wall was analyzed with DucoCell analysis program (Cellix Ltd). See table 3. FIG. 1A shows an example of platelet adhesion to collagen coated capillary under flow conditions in the presence or absence of a2b1 inhibitor (E180-413). FIG. 1B shows the dose dependent inhibitory effect of compound B1 on platelet adhesion.

TABLE 3
Analysis of inhibition of platelet adhesion to collagen coated
capillary under flow using Cellix microfluidic system.

| | Inhibition of |
|---|---|
| Compound | platelet aggregation |
| B94 | + |
| B93 | + |
| B40 | + |
| B165 | + |
| B67 | + |
| B78 | + |
| B60 | + |
| B42 | + |
| B33 | + |
| B19 | + |
| B11 | + |
| B54 | + |
| B3 | + |
| B1 | + |
| B167 | + |
| B7 | + |
| B17 | + |
| B109 | + |

+ indictes that the compound efficiently inhibited the platelet adhesion to collagen.

### A Platelet Function Analyzer PFA-100 was Used to Demonstrate the Anti-Thrombotic Potential of the α2β1 Inhibitors

A platelet function analyzer PFA-100 was used to demonstrate the possible antithrombotic effects of α2β1 modulators. The PFA-100 is a high shear-inducing device that simulates primary haemostasis after injury of a small vessel. The system comprises a test-cartridge containing a biologically active membrane coated with collagen and ADP. An anticoaculated whole blood sample was run through a capillary under a constant vacuum. The platelet agonist (ADP) on the membrane and the high shear rate resulted in activation of platelet aggregation, leading to occlusion of the aperture with a stable platelet plug. The time required to obtain full occlusion of the aperture was designated as the closure time. Each compound was added to the whole blood sample and the closure time was measured with PFA-100. If the closure time was increased when compared to the control sample the compound was suggested to have antithrombotic activity.

### Experimental Procedure

Blood was collected from a donor via venipuncture into evacuated blood collection Lithium heparin tubes (VenoJect, Terumo). Blood was aliquoted into 1.5 mL tubes and treated with either inhibitory compounds or controls (DMSO). Samples were kept at room temperature with rotation for 10 minutes and after that the closure time (Ct) of the blood was measured.

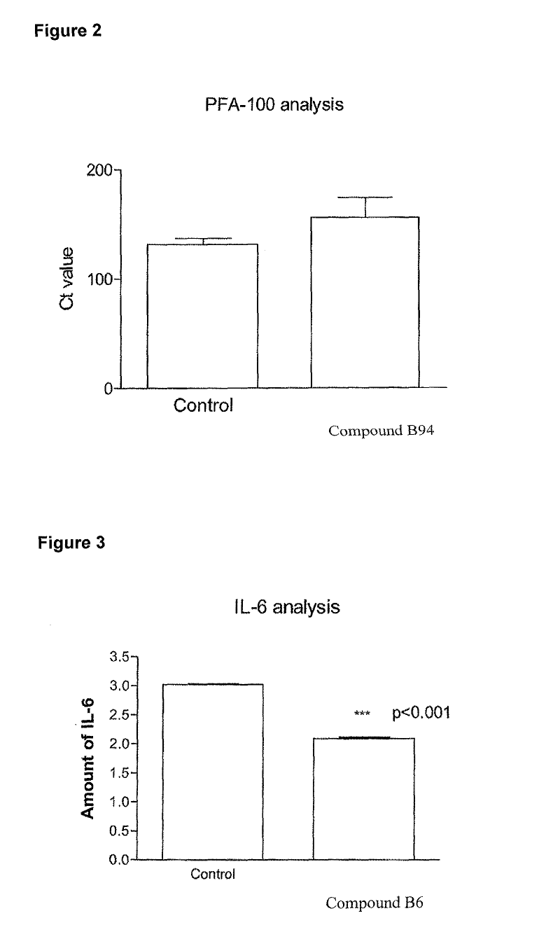

Results from the experiments show that the derivatives according to the invention increase the closure time (Ct) of the whole blood in PFA-100 analysis (see FIG. 2 for an example).

Interleukin-6 was measured to demonstrate the anti-inflammatory potential of the α2β1 inhibitors.

The inflammatory cells are shown to be dependent on α2β1 function during inflammatory process. The anti-inflammatory potential of the α2β1 modulators was studied by measuring the effect on the release of cytokines like IL-6 as an example from inflammatory cells. Lipopolysaccharide (LPS) was used to induce the production of IL-6. Anticoaculated whole blood sample was incubated with or without the integrin α2β1 inhibitor before LPS was added to induce the cytokine release. The amount of cytokine IL-6 released from peripheral blood leukocytes was measured from plasma samples 2 hours after the induction.

### Experimental Procedure

Blood was collected from the donors via venipuncture into evacuated Lithium heparin tubes (VenoJect, Terumo). Blood was treated with either α2β1 integrin inhibitors or vehicle control (DMSO). Samples were kept at room temperature for 5 minutes and lipopolysaccharide (LPS, 0.25 ng/mL) was added to induce cytokine release from peripheral blood lymphocytes. Samples were inculabted for 2 h in 37° C., the plasma was isolated and the amount of IL-6 was determined with Human IL-6 Quantikine ELISA Kit (R&D Systems).

Results from the experiments show that the derivatives according to the invention statistically significantly decrease the IL-6 release after incuction with LPS (see FIG. 3 for an example).

### Preparation of the Derivatives

### Experimental Procedure

¹H NMR spectra were recorded at ambient temperature using a Varian Unity Inova (400 MHz) spectrometer with a triple resonance 5 mm probe for example compounds, and either a Bruker Avance DRX (400 MHz) spectrometer or a Bruker Avance DPX (300 MHz) spectrometer for intermediate compounds. Chemical shifts are expressed in ppm relative to tetramethylsilane. The following abbreviations have been used: br=broad signal, s=singlet. d=doublet, dd=double doublet, dt=double triplet, t=triplet, q=quartet, m=multiplet.

High pressure liquid chromatography-Mass Spectrometry (LCMS) experiments to determine retention times (Rt) and associated mass ions were performed using one of the following methods:

Method A: Experiment performed on a Waters Platform LC quadrupole mass spectrometer linked to a Hewlett Packard HP1100 LC system with diode array detector and 100 position autosampler. The spectrometer has an electrospray source operating in positive and negative ion mode. Additional detection is achieved using a Sedex 85 evaporative light scattering detector. Samples are run through a LC column-Phenomenex Luna 3 micron C18(2) 30×4.6 mm and a 2 ml/min flow rate. The initial solvent system was 95% water containing 0.1% formic acid (solvent A) and 5% acetonitrile containing 0.1% formic acid (solvent B) for the first half minute followed by a gradient up to 5% solvent A and 95% solvent B over the next 4 minutes. The final solvent system was held constant for a further minute.

Method B: Experiment performed on a Waters ZMD quadrupole mass spectrometer linked to a Waters 1525 LC system with Waters 996 diode array detector. Sample injection is done by a Waters 2700 autosampler. The spectrometer has an electrospray source operating in positive and negative ion mode. Additional detection is achieved using a Sedex 85 evaporative light scattering detector. Samples are run through a LC column-Luna 3 micron C18(2) 30×4.6 mm and a 2 ml/min flow rate. The initial solvent system was 95% solvent A and 5% solvent B for the first half minute, followed by a gradient up to 5% solvent A and 95% solvent B over the next 4 minutes. The final solvent system was held constant for a further minute.

Method C: Experiment performed on a Waters Micromass ZQ2000 quadrupole mass spectrometer linked to a Hewlett Packard HP 1100 LC system with a DAD UV detector. Sample injection is done by a CTC HTS PAL autosampler. The spectrometer has an electrospray source operating in positive and negative ion mode. Additional detection is achieved using a Sedex 85 evaporative light scattering detector. Samples are run through a LC columnHiggins Clipeus 5 micron C18 100×3.0 mm and a 1 ml/min flow rate. The initial solvent system was 95% solvent A and 5% solvent B for the first minute, followed by a gradient up to 5% solvent A and 95% solvent B over the next 14 minutes. The final solvent system was held constant for a 5 further minutes.

Microwave experiments were carried out using a Biotage Initiator™, which uses a single-mode resonator and dynamic field tuning, both of which give reproducibility and control. Temperatures from 40-250° C. can be achieved, and pressures of up to 20 bars can be reached. Three types of vial are available for this processor, 0.5-2.0 ml, 2.0-5.0 ml and 5.0-20 ml.

Preparative HPLC purification was carried out using a C18-reversephase column (100×22.5 mm i.d. Genesis column with 7 μm particle size, UV detection at 230 or 254 nm, flow 5-15 ml/min), eluting with gradients from 100-0 to 0-100% water/acetonitrile containing 0.1% formic acid, with a flow rate of 18 ml per minute. Fractions containing the required product (identified by LCMS analysis) were pooled, the organic fraction removed by evaporation, and the remaining aqueous fraction lyophilised, to give the final product.

Compounds which required column chromatography were purified manually or fully automatically using either a Biotage SP1™ Flash Purification system with Touch Logic Control™ or a Combiflash Companion® with prepacked silica gel Isolute® SPE cartridge, Biotage SNAP cartridge or Redisep® Rf cartridge respectively.

Abbreviations:

DCM—Dichloromethane

DMF—N, N-Dimethylformamide

THF—Tetrahydrofuran

DMAP—4-Dimethylaminopyridine

TFA—Trifluoroacetic acid

Boc—tert-Butoxycarbonyl

IMS—Industrial methylated spirits

NMP—N-methylpyrrolidinone

### Intermediate 1

### (3-Methylaminophenyl)carbamic acid tert-butyl ester

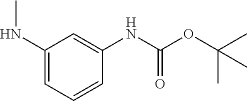

(3-Aminophenyl)carbamic acid tert-butyl ester (1.0 g) was dissolved in ethyl acetate (30 ml) and treated with aqueous formaldehyde (37% wt, 443 μl) and palladium on carbon (10%, 350 mg). The reaction mixture was hydrogenated under a balloon of hydrogen overnight at atmospheric pressure. The catalyst was removed by filtration through Celite under nitrogen and the volatiles were removed by evaporation. The residue was purified by chromatography using the Biotage system on a 10 g silica cartridge eluting with a mixture of ethyl acetate and cyclohexane (1:19 increasing to 1:1) to give (3-methylaminophenyl)carbamic acid tert-butyl ester (500 mg).

LCMS (Method A) Rt 2.45 (M+H⁺) 223

¹H NMR (300 MHz) (CDCl₃) δ 7.2 (t, 1H) 6.8 (br s, 1H) 6.5 (dd, 1H) 6.4 (br s, 1H), 6.3 (dd, 1H) 3.7 (br s, 1H) 2.8 (s, 3H) 1.5 (s, 9H)

### Intermediate 2

### {3-[N-(4′-Fluorobiphenyl-3-sulphonyl)-N-methylamino]phenyl}carbamic acid tert-butyl ester

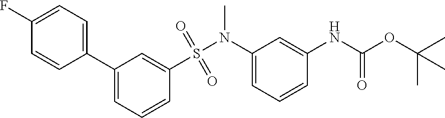

To a solution of 4′-fluorobiphenyl-3-sulphonyl chloride (245 mg) in pyridine (5 ml) was added a solution of (3-methylaminophenyl)carbamic acid tert-butyl ester (Intermediate 1, 200 mg) in pyridine (2 ml), and the resultant mixture was stirred at room temperature for 3 hours. The pyridine was removed by evaporation under reduced pressure and the residue was partitioned between water and ethyl acetate. The organic layer was dried (Na₂SO₄), filtered and the volatiles were removed by evaporation. The residue was purified by HPLC eluting with a mixture of water and acetonitrile (each containing 0.1% formic acid) from 20 to 98% acetonitrile over 25 minutes to give {3-[N-(4′-Fluorobiphenyl-3-sulphonyl)-N-methylamino]-phenyl}carbamic acid tert-butyl ester as a white solid (220 mg).

LCMS (Method A) Rt 4.26 (M−H) 455

¹H NMR (300 MHz) (CDCl₃) δ 7.75 (dd, 1H) 7.7 (br s, 1H) 7.6-7.5 (m, 2H) 7.5-7.4 (m, 2H) 7.35 (d, 1H) 7.2 (m, 2H) 7.15 (t, 2H) 6.75 (dd, 1H) 6.5 (br s, 1H) 3.2 (s, 3H) 1.5 (s, 9H)

By proceeding in a similar manner the following intermediates were prepared from the appropriate starting materials. The reaction may also be performed in DCM as a solvent in the presence of a base such as pyridine.

### Intermediate 3

### [4-(4′-Fluorobiphenyl-3-sulphonylamino)phenyl]carbamic acid tert-butyl

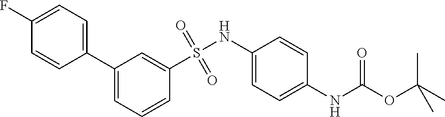

From 4′-fluorobiphenyl-3-sulphonyl chloride and (4-aminophenyl)carbamic acid tert-butyl ester

LCMS (Method A) Rt 4.09 (M−H) 441

¹H NMR (400 MHz) (CDCl₃) δ 7.8 (s, 1H) 7.7 (m, 2H) 7.5-7.4 (m, 3H) 7.2 (m, 3H) 7.0 (d, 2H) 6.6 (d, 2H) 6.4 (br s, 1H) 1.5 (s, 9H)

### Intermediate 4

### 4′-Fluorobiphenyl-3-sulphonic acid (4-nitrophenyl)amide

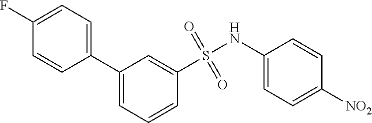

From 4′-fluorobiphenyl-3-sulphonyl chloride and 4-nitroaniline LCMS (Method A) Rt 3.88 (M−H) 371

¹H NMR (300 MHz) (CDCl₃) δ 8.2 (d, 2H) 8.1 (s, 1H) 7.8 (dd, 1H) 7.7 (dd, 1H) 7.6 (t, 1H) 7.5 (m, 2H) 7.2 (m, 3H) 7.1 (t, 2H)

### Intermediate 5

### 4′-Fluorobiphenyl-3-sulphonic acid (2-methoxy-5-nitrophenyl)amide

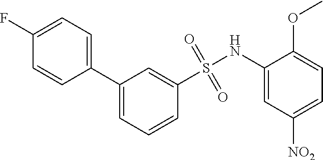

From 4′-fluorobiphenyl-3-sulphonyl chloride and 2-methoxy-5-nitroaniline

LCMS (Method A) Rt 3.86 (M−H) 401

### Intermediate 6

### 5-Bromopyridine-3-sulphonic acid N-methyl-N-[4-(3-phenylureido)phenyl]amide

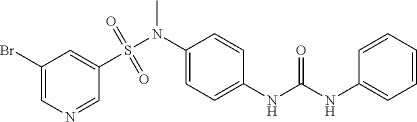

From 5-bromopyridine-3-sulphonyl chloride and 1-(4-methylaminophenyl)-3-phenyl urea (Intermediate 25)

LCMS (Method A) Rt 3.47 (M+H⁺) 338

### Intermediate 7

### 3-Bromophenylsulphonic acid N-methyl-N-[4-(3-phenylureido)phenyl]amide

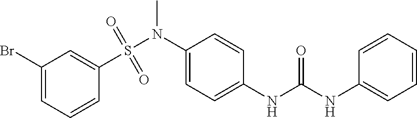

From 3-bromophenylsulphonyl chloride and 1-(4-methylaminophenyl)-3-phenyl urea (Intermediate 25)

LCMS (Method A) Rt. 3.87 (M+H⁺) 462

¹H NMR (300 MHz) (DMSO-d₆) δ 8.8 (s, 1H) 8.7 (s, 1H) 7.9 (dd, 1H) 7.6 (m, 3H) 7.4 (m, 4H) 7.3 (t, 2H) 7.0 (m, 3H) 3.1 (s, 3H)

### Intermediate 8

### 1-(4-Fluorophenyl)-1H-pyrazole-4-sulphonic acid [4-(3-phenylureido)phenyl]amide

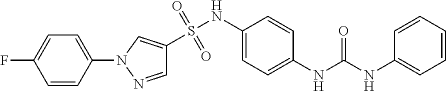

From 1-(4-fluorophenyl)-1H-pyrazole-4-sulphonyl chloride (Intermediate 87) and 1-(4-aminophenyl)-3-phenyl urea (Intermediate 23)

LCMS (Method C) Rt 10.56 (M+H⁺) 452

¹H NMR (400 MHz) (DMSO-d₆) δ 10.0 (br s, 1H) 8.9 (s, 1H) 8.6 (br s, 2H) 7.9 (m, 3H) 7.4 (m, 6H) 7.3 (t, 2H) 7.1 (d, 2H) 6.9 (t, 1H)

### Intermediate 9

### 5-(4,4,4-Trifluoro-1,3-dioxobutyl)thiophene-2-sulphonic acid N-methylN-[4-(3-phenyl-ureido)phenyl]amide

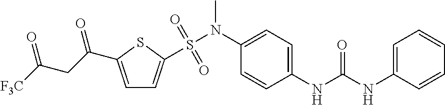

From 5-(4,4,4-trifluoro-1,3-dioxobutyl)thiophene-2-sulphonyl chloride and 1-(4-methyl-aminophenyl)-3-phenylurea (Intermediate 25)

Intermediate used without purification or characterisation

### Intermediate 10

### 4′-Fluorobiphenyl-3-sulphonic acid N-methyl-N-(4-nitrophenyl)amide

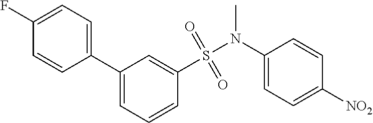

From 4′-fluorobiphenyl-3-sulphonyl chloride and N-methyl-4-nitroaniline using pyridine in DCM.

¹H NMR (400 MHz) (CDCl₃) δ 8.2 (d, 2H) 7.75 (dt, 1H) 7.70 (t, 1H) 7.5 (t, 1H) 7.45 (m, 3H) 7.35 (d, 2H) 7.15 (t, 2H) 3.3 (s, 3H)

### Intermediate 11

### 5-(1-Methyl-3-trifluoromethyl-1H-pyrazol-5-yl)thiophene-2-sulphonic acid (2-methoxy-4-nitrophenyl)amide

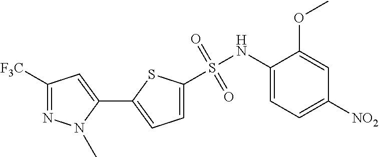

From 5-(1-methyl-3-trifluoromethyl-1H-pyrazol-5-yl)thiophene-2-sulphonyl chloride and 2-methoxy-4-nitroaniline using pyridine in DCM.

¹H NMR (400 MHz) (DMSO-d₆) δ 10.7 (br s, 1H) 7.9 (dd, 1H) 7.8 (d, 1H) 7.75 (d, 1H) 7.65 (d, 1H) 7.55 (d, 1H) 7.2 (s, 1H) 4.0 (s, 3H) 3.8 (s, 3H)

### Intermediate 12

### 4′-Fluorobiphenyl-3-sulphonic acid (2-methyl-4-nitrophenyl)amide

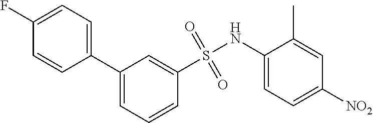

From 4′-fluorobiphenyl-3-sulphonyl chloride and 2-methyl-4-nitroaniline using pyridine in DCM.

¹H NMR (300 MHz) (CDCl₃) δ 8.2 (d, 1H) 8.0 (t, 1H) 7.9 (dd, 1H) 7.7 (m, 2H) 7.6 (t, 1H) 7.5 (m, 2H) 7.3 (d, 1H) 7.2 (t, 2H) 6.5 (br s, 1H) 2.2 (s, 3H)

### Intermediate 13

### 4′-Fluorobiphenyl-3-sulphonic acid (2-methoxy-4-nitrophenyl)amide

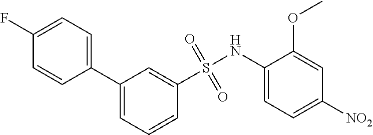

From 4′-fluorobiphenyl-3-sulphonyl chloride and 2-methoxy-4-nitroaniline using pyridine in DCM.

¹H NMR (400 MHz) (CDCl₃) δ 8.0 (t, 1H) 7.85 (m, 2H) 7.75 (dt, 1H) 7.66 (s, 1H) 7.64 (d, 1H) 7.55 (t, 1H) 7.48 (m, 3H) 7.15 (t, 2H) 3.8 (s, 3H)

### Intermediate 14

### 4-Nitrophenylsulphonic acid (3-bromophenyl)amide

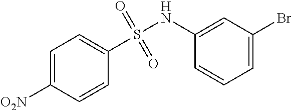

From 4-nitrophenylsulphonyl chloride and 3-bromoaniline using pyridine in DCM.

¹H NMR (300 MHz) (DMSO-d₆) δ 10.90 (br s, 1H), 8.40 (d, 2H) 8.02 (d, 2H) 7.31-7.21 (m, 3H) 7.13 (dt, 1H).

### Intermediate 15

### 4-Bromo-5-chlorothiophene-2-sulphonic acid N-methyl-N-[4-(3-phenylureido)phenyl]amide

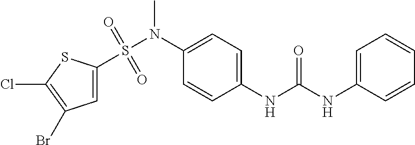

From 4-bromo-5-chlorothiophene-2-sulphonyl chloride and 1-(4-methylaminophenyl)-3-phenyl urea, (Intermediate 25)

¹H NMR (300 MHz) (DMSO-d₆) δ 8.8 (br s, 1H), 8.7 (br s, 1H) 7.65 (s, 1H) 7.5 (dd, 4H) 7.3 (t, 2H) 7.15 (d, 2H) 7.0 (t, 1H) 3.2 (s, 3H)

### Intermediate 16

### 1H-Imidazole-4-sulphonic acid N-methyl-N-[4-(3-phenylureido)phenyl]amide

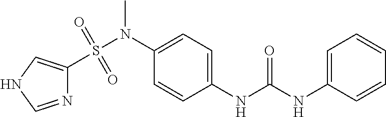

From 1-H-imidazole-4-sulphonyl chloride and 1-(4-methylaminophenyl)-3-phenyl urea (Intermediate 25) using pyridine in DCM.

LCMS (Method A) Rt 2.90 (M+H⁺) 372

### Intermediate 17

### 1H-Pyrazole-4-sulphonic acid N-methyl-N-[4-(3-phenylureido)phenyl]amide

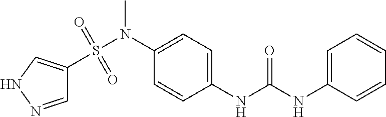

From 1H-pyrazole-4-sulphonyl chloride and 1-(4-methylaminophenyl)-3-phenyl urea (Intermediate 25) using pyridine in DCM.

LCMS (Method C) Rt 8.55 (M+H⁺) 372

¹H NMR (400 MHz) (DMSO-d₆) 8.85 (s, 1H), 8.75 (s, 1H) 7.9 (s, 2H) 7.4 (m, 4H) 7.25 (t, 2H) 7.0 (d, 2H) 6.95 (t, 1H) 3.1 (s, 3H).

### Intermediate 18

### 4′-Fluorobiphenyl-3-sulphonic acid N-[(2,2-dimethyl-1,3-dioxolan-4-yl)methyl]-N-(4-nitrophenyl)amide

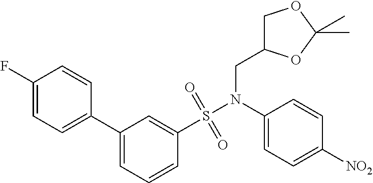

To a stirred solution of N-[(2,2-dimethyl-1,3-dioxolan-4-yl)methyl]-4-nitroaniline (Intermediate 19, 300 mg) in DMF (9 ml) was added portionwise NaH (60% in mineral oil, 160 mg). The mixture was stirred for 15 minutes and then a solution of 4′-fluorobiphenyl-3-sulphonyl chloride (390 mg) in DMF (1 ml) was added. Stirring was continued for a further 4 hours then the reaction mixture was quenched with water and extracted with ethyl acetate. The organic layer was washed with water, dried (Na₂SO₄), filtered and the volatiles were removed by evaporation. The residue was purified by chromatography using the Companion on a 20 g silica cartridge, eluting with a mixture of ethyl acetate and cyclohexane (1:9 increasing to 1:4), to give 4′-fluorobiphenyl-3-sulphonic acid N-[(2,2-dimethyl-1,3-dioxolan-4-yl)methyl]-N-(4-nitrophenyl)amide as a yellow oil (320 mg).

¹H NMR (300 MHz) (DMSO-d₆) δ 8.2 (d, 2H) 7.8 (dt, 1H) 7.75 (t, 1H) 7.55 (t, 1H) 7.5 (m, 3H) 7.35 (d, 2H) 7.15 (m, 2H) 4.25 (m, 1H) 4.05 (dd, 1H) 3.8 (dd, 1H) 3.75 (dd, 2H) 1.25 (s, 6H)

### Intermediate 19

### N-[(2,2-Dimethyl-1,3-dioxolan-4-yl)methyl]-4-nitroaniline

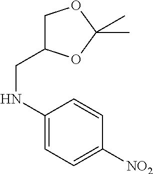

A solution of (2,2-dimethyl-1,3-dioxolan-4-yl)methylamine (2.5 ml), 1-fluoro-4-nitrobenzene (1.41 g) and triethylamine (1.4 ml) in ethanol (25 ml) was heated under reflux for 18 hours. The reaction mixture was cooled and the volatiles were removed by evaporation. The residue was purified by chromatography using the Companion on a 50 g silica cartridge, eluting with a mixture of ethyl acetate and pentane (1:4) to give N-[(2,2-dimethyl-1,3-dioxolan-4-yl)methyl]-4-nitroaniline as a yellow solid (533 mg).

¹H NMR (400 MHz) (CDCl₃) δ 8.1 (d, 2H) 6.6 (d, 2H) 4.8 (br s, 1H) 4.4 (m, 1H) 4.1 (dd, 1H) 3.8 (dd, 1H) 3.4 (m, 1H) 3.3 (m, 1H) 1.5 (s, 3H) 1.4 (s, 3H)

### Intermediate 20

### N-Methyl-N-(4-nitrophenyl)carbamic acid tert-butyl ester

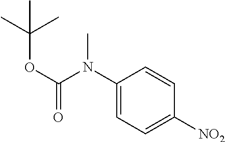

N-Methyl-4-nitroaniline (1.5 g) was dissolved in THF (100 ml) and boc anhydride was slowly added followed by DMAP. The mixture was stirred at room temperature for 4 hours then the volatiles were removed by evaporation. The residue was partitioned between water and ethyl acetate and the organic layer was washed with aqueous HCl, dried (Na₂SO₄) and filtered. The volatiles were removed by evaporation to give N-methyl-N-(4-nitrophenyl)carbamic acid tert-butyl ester as a pale beige solid (2.29 g)

LCMS (Method A) Rt 3.84 (M+H⁺+CH3CN) 294

¹H NMR (400 MHz) (CDCl₃) δ 8.2 (d, 2H) 7.5 (d, 2H) 3.3 (s, 3H) 1.5 (s, 9H)

### Intermediate 21

### 4′-Fluorobiphenyl-3-sulphonic acid N-(3-aminophenyl)-N-methylamide

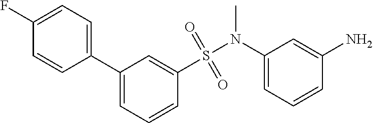

{3-[N-(4′-Fluorobiphenyl-3-sulphonyl)-N-methylamino]phenyl}carbamic acid tert-butyl ester (Intermediate 2, 200 mg) was dissolved in DCM (6 ml) and treated with TFA (2 ml). The mixture was stirred at room temperature for 2 hours, then the volatiles were removed by evaporation. The residue was partitioned between saturated aqueous NaHCO₃ and ethyl acetate and the organic layer was dried (Na₂SO₄), filtered and the volatiles were removed by evaporation to give 4′-fluorobiphenyl-3-sulphonic acid N-(3-aminophenyl)-N-methylamide (169 mg).

LCMS (Method B) Rt 3.72 (M+H⁺) 357

¹H NMR (300 MHz) (CDCl₃) δ 7.7 (m, 2H) 7.6-7.4 (m, 4H) 7.2 (m, 3H) 6.6 (m, 2H) 6.4 (dd, 1H) 3.7 (br s, 2H) 3.3 (s, 3H)

By proceeding in a similar manner the following intermediates were prepared from the appropriate starting materials:

### Intermediate 22

### 4′-Fluorobiphenyl-3-sulphonic acid (4-aminophenyl)amide

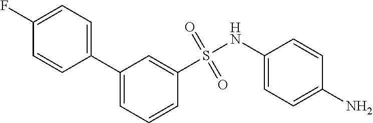

From {4-[N-(4′-fluorobiphenyl-3-sulphonyl)amino]phenyl}carbamic acid tert-butyl ester (Intermediate 3)

LCMS (Method B) Rt 2.95 (M+H⁺) 343

¹H NMR (300 MHz) (CDCl₃) δ 7.8 (s, 1H) 7.7 (m, 2H) 7.5 (m, 3H) 7.1 (t, 2H) 6.8 (d, 2H) 6.6 (m, 4H) 6.1 (s, 1H)

### Intermediate 23

### 1-(4-Aminophenyl)-3-phenyl urea

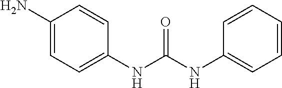

From N-[4-(3-phenylureido)phenyl]carbamic acid tert-butyl ester (Intermediate 44)

LCMS (Method B) Rt 0.32 & 1.84 (M+H⁺) 228

¹H NMR (300 MHz) (DMSO-d₆) δ 8.5 (br s, 1H) 8.1 (br s, 1H) 7.4 (d, 2H) 7.3 (t, 2H) 7.1 (d, 2H) 6.9 (t, 1H) 6.5 (d, 2H) 4.8 (br s, 2H)

### Intermediate 24

### 1-(3-Aminophenyl)-3-phenyl urea

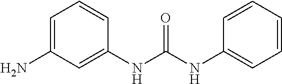

From N-[3-(3-phenylureido)phenyl]carbamic acid tert-butyl ester (Intermediate 37)

¹H NMR (400 MHz) (DMSO-d₆) δ 8.5 (s, 1H) 8.3 (s, 1H) 7.4 (d, 2H) 7.25 (t, 2H) 6.95 (t, 1H) 6.85 (t, 1H) 6.75 (t, 1H) 6.55 (dd, 1H) 6.2 (dd, 1H) 5.0 (br s, 2H)

### Intermediate 25

### 1-(4-Methylaminophenyl)-3-phenyl urea

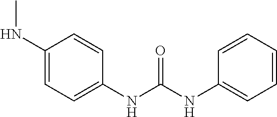

From N-methyl-N-[4-(3-phenylureido)phenyl]carbamic acid tert-butyl ester (Intermediate 36)

LCMS (Method B) Rt 1.87 (M+H⁺) 242

¹H NMR (400 MHz) (DMSO-d₆) δ 8.9 (br s, 2H) 7.5 (m, 4H) 7.3 (t, 2H) 7.0 (d, 2H) 6.9 (t, 1H) 4.0 (br s, 1H) 2.8 (s, 3H)

### Intermediate 26

### 1-(4-Aminophenyl)-3-benzyl urea

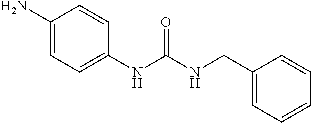

From N-[4-(3-benzylureido)phenyl]carbamic acid tert-butyl ester (Intermediate 41)

LCMS (Method A) Rt 0.37 & 1.96 (M+H⁺) 242

### Intermediate 27

### 1-(4-Amino-3-methylphenyl)-3-phenyl urea

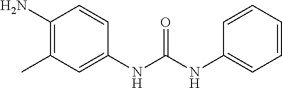

To a suspension of 1-[4-(5-chloro-1,3-dioxo-1,3-dihydroisoindol-2-yl)-3-methylphenyl]-3-phenyl urea (Intermediate 56, 811 mg) in ethanol (10 ml) was added hydrazine hydrate (2 ml). The mixture was stirred and heated under reflux for 20 minutes, then the volatiles were removed by evaporation. The residue was redissolved in ethyl acetate (10 ml) and heated under reflux for further 20 minutes. The reaction mixture was cooled, filtered and the filtrate was concentrated. The residue was purified by HPLC eluting with a mixture of water and acetonitrile (each containing 0.1% formic acid) from 30 to 60% over 20 minutes to give 1-(4-amino-3-methylphenyl)-3-phenyl urea as a white powder.

LCMS (Method A) Rt 2.03 (M+H⁺) 242

### Intermediate 28

### 4′-Fluorobiphenyl-3-sulphonic acid N-(3-benzyloxypropyl)-N-(4-nitrophenyl)amide

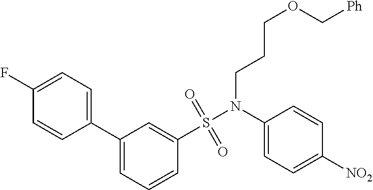

4′-Fluorobiphenyl-3-sulphonic acid N-(4-nitrophenyl)amide (Intermediate 4, 200 mg) was dissolved in THF (4.0 ml) and triphenyl phosphine (280 mg) was added, followed by 3-benzyloxypropan-1-ol (170 μl). The mixture was then cooled to 5° C. and diethyl azodicarboxylate (168 μl) was slowly added. The reaction mixture was stirred overnight at room temperature. The volatiles were removed by evaporation and the residue was purified by chromatography on a 5 g silica cartridge eluting with a mixture of ethyl acetate and cyclohexane (1:4) to give 4′-fluorobiphenyl-3-sulphonic acid N-(3-benzyloxypropyl)-N-(4-nitrophenyl)amide as a yellow oil (236 mg).

¹H NMR (300 MHz) (CDCl₃) δ 8.4 (d, 2H) 7.8 (m, 2H) 7.5-7.4 (m, 4H) 7.4-7.2 (m, 7H) 7.1 (t, 2H) 4.4 (s, 2H) 3.8 (t, 2H) 3.5 (t, 2H) 1.8 (t, 2H)

By proceeding in a similar manner the following compounds were prepared from the appropriate starting materials:

### Intermediate 29

### 4′-Fluorobiphenyl-3-sulphonic acid N-(3-tert-butoxypropyl)-N-(2-methoxy-4-nitrophenyl)-amide

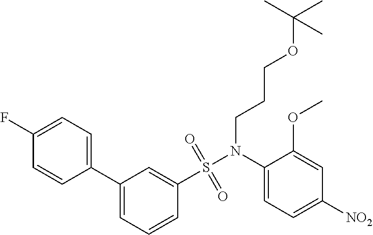

From 4′-fluorobiphenyl-3-sulphonic acid (2-methoxy-4-nitrophenyl)amide (Intermediate 13) and 3-tert-butoxypropan-1-ol

¹H NMR (400 MHz) (DMSO-d₆) δ 8.0 (d, 1H) 7.9 (dd, 1H) 7.8 (t, 1H) 7.75 (d, 1H) 7.7 (m, 3H) 7.65 (d, 1H) 7.5 (d, 1H) 7.3 (t, 2H) 3.65 (t, 2H) 3.5 (s, 3H) 3.2 (t, 2H) 1.45 (m, 2H) 1.1 (s, 9H)

### Intermediate 30

### 3-(2-Methoxyethoxy)nitrobenzene

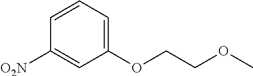

From 3-nitrophenol and 2-methoxyethanol

¹H NMR (300 MHz) (DMSO-d₆) δ 7.9 (dd, 1H) 7.8 (d, 1H) 7.4 (t, 1H) 7.3 (dd, 1H) 4.2 (t, 2H) 3.7 (t, 2H) 3.5 (s, 3H)

### Intermediate 31

### [N-(4′-Fluorobiphenyl-3-sulphonyl)-N-(4-nitrophenyl)amino]acetic acid ethyl ester

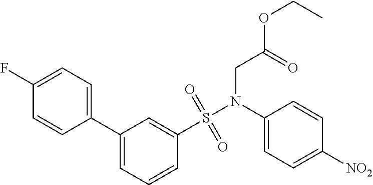

A solution of 4′-fluorobiphenyl-3-sulphonic acid (4-nitrophenyl)amide (Intermediate 4, 100 mg) in THF (3 ml) was added to an ice-cooled suspension of NaH (60% in mineral oil, 14 mg) in THF (2 ml). The mixture was stirred for 15 minutes at 0° C. and then treated with ethyl bromoacetate (83 μl). The resultant mixture was stirred at room temperature for 2.5 hours followed by heating at 50° C. overnight. The volatiles were removed by evaporation and the residue was diluted with water, acidified with aqueous HCl and extracted with ethyl acetate. The organic layer was dried (Na₂SO₄), filtered and the volatiles were removed by evaporation. The reaction was repeated using 4′-fluorobiphenyl-3-sulphonic acid (4-nitrophenyl)amide (Intermediate 4, 601 mg) in THF (15 ml), NaH (84 mg) in THF (30 ml) and ethyl bromoacetate (500 μl). The crude residues from both experiments were combined and purified by chromatography using the Biotage system on a 10 g silica cartridge eluting with a mixture of ethyl acetate and cyclohexane (1:9) to give [N-(4′-fluorobiphenyl-3-sulphonyl)-N-(4-nitrophenyl)amino]acetic acid ethyl ester (780 mg).

¹H NMR (300 MHz) (CDCl₃) δ 8.2 (d, 2H) 7.9 (s, 1H) 7.8 (dd, 1H) 7.7 (dd, 1H) 7.6-7.4 (m, 5H) 7.1 (t, 2H) 4.5 (s, 2H) 4.2 (q, 2H) 1.2 (t, 3H)

By proceeding in a similar manner the following compound was prepared from the appropriate starting materials:

### Intermediate 32

### 4′-Fluorobiphenyl-3-sulphonic acid N-methyl-N-(2-methyl-4-nitrophenyl)amide

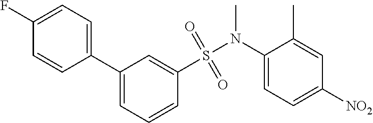

From 4′-fluorobiphenyl-3-sulphonic acid (2-methyl-4-nitrophenyl)amide (Intermediate 12) and iodomethane

LCMS (Method B) Rt 3.98 (M−H) 401

### Intermediate 33

### 5-[1-(Tetrahydropyran-2-yl)-1H-pyrazol-5-yl]thiophene-2-sulphonic acid N-methyl-N-[4-(3-phenylureido)phenyl]amide

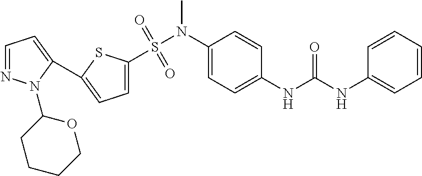

A mixture of 5-bromothiophene-2-sulphonic acid N-methyl-N-[4-(3-phenylureido)phenyl]-amide (Example 55, 93 mg), 1-(tetrahydropyran-2-yl)-1H-pyrazole-5-boronic acid pinacol ester (48 mg), tetrakis(triphenylphosphine)palladium(0) (14 mg), Na₂CO₃ (2M, 1.3 ml) and DME (2 ml) was heated in the microwave at 150° C. for 20 minutes. The resultant mixture was partitioned between water and ethyl acetate. The organic layer was dried (MgSO₄), filtered and the volatiles were removed by evaporation. The residue was purified by chromatography on a 5 g silica cartridge eluting with a mixture of ethyl acetate and DCM (1:49 increasing to 1:9) to give 5-[1-(tetrahydropyran-2-yl)-1H-pyrazol-5-yl]thiophene-2-sulphonic acid N-methyl-N-[4-(3-phenylureido)phenyl]-amide (62 mg).

LCMS (Method B) Rt 3.24 (M+H⁺) 376

By proceeding in a similar manner the following intermediates were prepared from the appropriate starting materials. The reaction may also be performed using different catalysts, bases and solvents.

### Intermediate 34

### 4-Nitrophenylsulphonic acid (4′-fluorobiphenyl-3-yl)amide

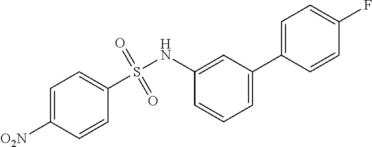

From 4-fluorophenylboronic acid and 4-nitrophenylsulphonic acid (3-bromophenyl)amide (Intermediate 14) using [1,1-Bis(diphenylphosphino)ferrocene]dichloropalladium (II) and Cs₂CO₃, in a mixture of DME and IMS.

The compound was used without purification or characterisation.

### Intermediate 35

### 5-(1-Boc-3,6-dihydro-2H-pyridin-4-yl)thiophene-2-sulphonic acid N-methyl-N-[4-(3-phenyl-ureido)phenyl]amide

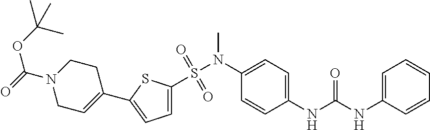

From 5-bromothiophene-2-sulphonic acid N-methyl-N-[4-(3-phenylureido)phenyl]-amide (Example 55) and 1-boc-3,6-dihydro-2H-pyridine-4-boronic acid pinacol ester.

LCMS (Method B) Rt 4.26 (M+H⁺) 569

### Intermediate 36

### N-Methyl-N-[4-(3-phenylureido)phenyl]carbamic acid tert-butyl ester

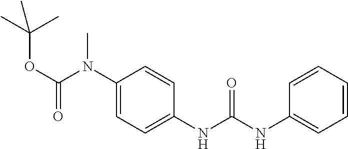

N-(4-Aminophenyl)-N-methylcarbamic acid tert-butyl ester (Intermediate 62, 1.80 g) was dissolved in THF (50 ml) and treated with NaOH (1M, 8.5 ml) and phenyl isocyanate (715 μl). The resultant mixture was stirred at room temperature for 2 hours and the volatiles were removed by evaporation. The residue was acidified to pH 5 and extracted with ethyl acetate. The organic layer was dried (Na₂SO₄), filtered and the volatiles were removed by evaporation. The residue was purified by chromatography using the Biotage system on a 50 g silica cartridge eluting with a mixture of ethyl acetate and cyclohexane (1:19 increasing to 2:3) to give N-methyl-N-[4-(3-phenylureido)phenyl]carbamic acid tert-butyl ester as a white solid (2.74 g).

LCMS (Method B) Rt 3.64 (M+H⁺) 340

¹H NMR (400 MHz) (CDCl₃) δ 7.4 (m, 4H) 7.2 (m, 1H) 7.1 (m, 4H) 6.9 (br s, 1H) 6.8 (br s, 1H) 3.2 (s, 3H) 1.5 (s, 9H)

By proceeding in a similar manner the following compounds were prepared from the appropriate starting materials:

### Intermediate 37

### N-[3-(3-Phenylureido)phenyl]carbamic acid tert-butyl ester

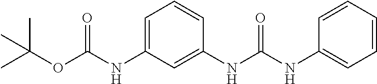

From N-(3-aminophenyl)carbamic acid tert butyl ester and phenyl isocyanate

LCMS (Method B) Rt 3.65 (M+H⁺) 328

### Intermediate 38

### 4′-Fluorobiphenyl-3-sulphonic acid N-(3-tert-butoxypropyl)-N-[2-methoxy-4-(3-phenyl-ureido)phenyl]amide

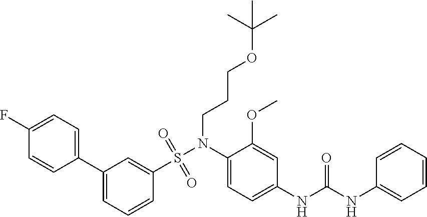

From 4′-fluorobiphenyl-3-sulphonic acid N-(3-tert-butoxypropyl)-N-(2-methoxy-4-amino-phenyl)amide (Intermediate 75) and phenyl isocyanate

¹H NMR (400 MHz) (DMSO-d₆) δ 8.8 (s, 1H) 8.7 (s, 1H) 7.95 (d, 1H) 7.7 (m, 5H) 7.45 (d, 2H) 7.3 (m, 5H) 7.05 (d, 1H) 7.0 (t, 1H) 6.9 (dd, 1H) 3.6 (br s, 2H) 3.3 (m, 5H) 1.5 (m, 2H) 1.05 (s, 9H)

### Intermediate 39

### {N-(4′-Fluorobiphenyl-3-sulphonyl)-N-[4-(3-phenylureido)phenyl]-amino}acetic acid ethyl ester

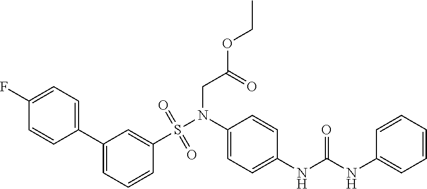

N-(4-Aminophenyl)-N-(4′-fluorobiphenyl-3-sulphonyl)amino]acetic acid ethyl ester (Intermediate 60, 410 mg) was dissolved in THF (14 ml) and treated with phenyl isocyanate (114 μl). The mixture was stirred and heated at 70° C. for 6 hours. The volatiles were removed by evaporation and the residue was partitioned between saturated aqueous NaHCO₃ and ethyl acetate. The organic layer was dried (Na₂SO₄), filtered and the filtrate was concentrated to dryness. The residue was purified by chromatography using a 10 g silica cartridge eluting with a mixture of ethyl acetate and cyclohexane (1:9) to give {N-(4′-fluorobiphenyl-3-sulphonyl)-N-[4-(3-phenylureido)phenyl]amino}acetic acid ethyl ester (280 mg).

LCMS (Method B) Rt 4.22 (M+H⁺) 548

¹H NMR (300 MHz) (DMSO-d₆) δ 8.8 (s, 1H), 8.7 (s, 1H) 8.0 (dd, 1H) 7.8 (s, 1H) 7.7-7.6 (m, 4H) 7.4-7.2 (m, 8H) 7.1 (d, 2H) 7.0 (t, 1H) 4.5 (s, 2H) 4.1 (q, 2H) 1.1 (t, 3H)

By proceeding in a similar manner the following compounds were prepared from the appropriate starting materials. The reaction may also be performed using ethyl acetate or toluene as solvent, either at room temperature or at reflux.

### Intermediate 40

### 1-(4-Nitrophenyl)-3-pyridin-4-yl urea

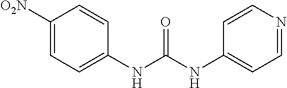

From 4-nitrophenyl isocyanate and 4-aminopyridine

¹H NMR (400 MHz) (DMSO-d₆) δ 9.7 (br s, 1H) 9.3 (br s, 1H) 8.4 (m, 2H) 8.2 (d, 2H) 7.7 (d, 2H) 7.4 (m, 2H)

### Intermediate 41

### N-[4-(3-Benzylureido)phenyl]carbamic acid tert-butyl ester

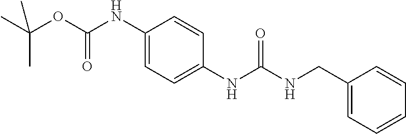

From benzyl isocyanate and (4-aminophenyl)carbamic acid tert-butyl ester

LCMS (Method A) Rt 3.46 (M+H⁺) 342

### Intermediate 42

### 1-[3-(2-Methoxyethoxy)phenyl]-3-(4-nitrophenyl)urea

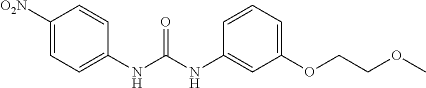

From 4-nitrophenyl isocyanate and 3-(2-methoxyethoxy)aniline (Intermediate 70)

LCMS (Method A) Rt 3.42 (M+H⁺) 332

¹H NMR (400 MHz) (CDCl₃) δ 8.2 (d, 2H) 7.5 (d, 2H) 7.4 (br s, 1H) 7.2 (d, 1H) 7.0 (br s, 1H) 6.9 (m, 2H) 6.6 (m, 1H) 4.1 (m, 2H) 3.8 (m, 2H) 3.4 (s, 3H)

### Intermediate 43

### 4′-Fluorobiphenyl-3-sulphonic acid N-(3-benzyloxypropyl)-N-[4-(3-phenylureido)phenyl]-amide

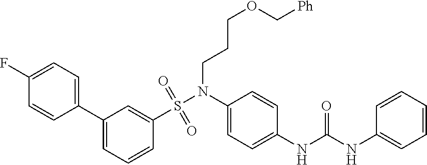

From 4-fluorobiphenyl-3-sulphonic acid N-(3-benzyloxypropyl)-N-(4-aminophenyl)amide (Intermediate 84) and phenyl isocyanate

LCMS (Method A) Rt 4.44 (M+H⁺) 610

### Intermediate 44

### N-[4-(3-Phenylureido)phenyl]carbamic acid tert-butyl ester

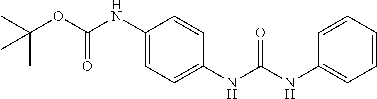

From (4-aminophenyl)carbamic acid tert-butyl ester and phenyl isocyanate

LCMS (Method A) Rt 3.52 (M+H⁺) 328

¹H NMR (300 MHz) (DMSO-d₆) δ 9.2 (br s, 1H) 8.6 (br s, 1H), 8.5 (br s, 1H) 7.4 (d, 2H) 7.3 (m, 6H), 6.9 (t, 1H) 1.5 (s, 9H)

### Intermediate 45

### 4′-Fluorobiphenyl-3-sulphonic acid N-[(2,2-dimethyl-1,3-dioxolan-4-yl)methyl]-N-[4-(3-phenylureido)phenyl]amide

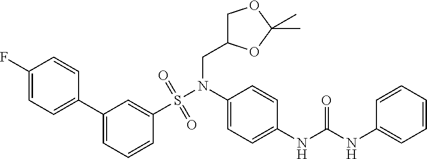

From 4′ fluorobiphenyl-3-sulphonic acid N-[(2,2-dimethyl-1,3-dioxolan-4-yl)methyl]-N-(4-aminophenyl)amide (Intermediate 78) and phenyl isocyanate

LCMS (Method B) Rt 4.25 (M+H⁺) 576

### Intermediate 46

### 1-(4-Methyl-3-nitrophenyl)-3-phenyl urea

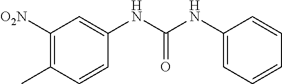

From 4-methyl-3-nitrophenyl isocyanate and aniline in ethyl acetate at room temperature

¹H NMR (400 MHz) (DMSO-d₆) δ 9.00 (s, 1H) 8.7 (s, 1H) 8.2 (s, 1H) 7.4 (m, 6H) 6.95 (s, 1H) 2.45 (s, 3H)

### Intermediate 47

### 1-(2-Chloro-5-nitrophenyl)-3-phenyl urea

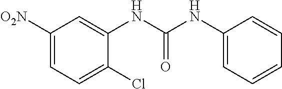

From 2-chloro-5-nitrophenyl isocyanate and aniline in ethyl acetate at room temperature

¹H NMR (400 MHz) (DMSO-d₆) δ 9.6 (s, 1H) 9.2 (s, 1H) 8.7 (s, 1H) 7.8 (d, 2H) 7.4 (d, 4H) 7.0 (s, 1H)

### Intermediate 48

### 1-(4-Chloro-3-nitrophenyl)-3-phenyl urea

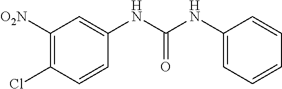

From 4-chloro-3-nitrophenyl isocyanate and aniline in ethyl acetate at room temperature

¹H NMR (400 MHz) (DMSO-d₆) δ 9.2 (s, 1H) 8.9 (s, 1H) 8.3 (s, 1H) 7.6 (m, 2H) 7.5 (d, 2H) 7.3 (t, 2H) 7.0 (t, 1H)

### Intermediate 49

### 1-(2-Chloro-4-nitrophenyl)-3-phenyl urea

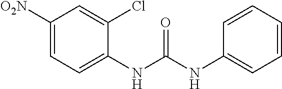

From 2-chloro-4-nitrophenyl isocyanate and aniline in ethyl acetate at room temperature

¹H NMR (400 MHz) (DMSO-d₆) δ 9.7 (s, 1H) 8.8 (s, 1H) 8.55 (d, 1H) 8.35 (s, 1H) 8.2 (d, 1H) 7.5 (d, 2H) 7.3 (t, 2H) 7.0 (t, 1H)

### Intermediate 50

### 1-(4-Methyl-3-nitrophenyl)-3-phenyl urea

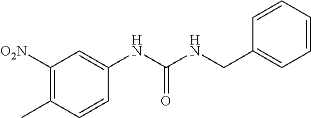

From 4-methyl-3-nitrophenyl isocyanate and benzylamine in ethyl acetate at room temperature

LCMS (Method A) Rt 3.46 (M+H⁺) 286

¹H NMR (400 MHz) (DMSO-d₆) □ 8.95 (s, 1H) 8.25 (s, 1H) 7.5 (d, 1H) 7.3 (m, 5H) 7.2 (m, 1H) 6.75 (t, 1H) 4.3 (d, 2H) 2.4 (s, 3H)

### Intermediate 51

### 1-(4-Nitrophenyl)-3-[2-(pyridin-3-yl)ethyl]urea

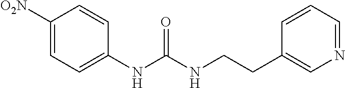

From 4-nitrophenyl isocyanate and 2-(pyridin-3-yl)ethylamine in ethyl acetate at room temperature.

LCMS (Method A) Rt 1.97 (M+H⁺) 287

### Intermediate 52

### 1-(3-Methyl-4-nitrophenyl)-3-(2-methylphenyl)urea

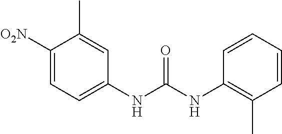

From 3-methyl-4-nitrophenyl isocyanate and 2-methylaniline in ethyl acetate at room temperature.

LCMS (Method A) Rt 3.63 (M+H⁺) 286

### Intermediate 53

### 6-Nitro-2,3-dihydro-1H-indole-1-carboxylic acid N-phenylamide

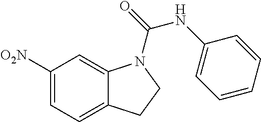

From 6-nitro-2,3-dihydro-1H-indole and phenyl isocyanate in ethyl acetate at room temperature.

LCMS (Method A) Rt 3.56 (M+H⁺) 284

### Intermediate 54

### 1-(4-Nitrophenyl)-3-(1-phenylethyl)urea

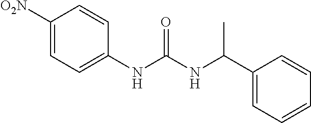

From 4-nitrophenyl isocyanate and 1-phenylethylamine in ethyl acetate at room temperature.

LCMS (Method A) Rt 3.48 (M+H⁺) 286

### Intermediate 55

### 1-(2-Methoxy-5-nitrophenyl)-3-phenyl urea

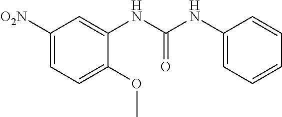

From 2-methoxy-5-nitrophenyl isocyanate and aniline in ethyl acetate at room temperature

LCMS (Method A) Rt 3.57 (M+H⁺) 388

### Intermediate 56

### 1-[4-(5-Chloro-1,3-dioxo-1,3-dihydroisoindol-2-yl)-3-methylphenyl]-3-phenyl urea

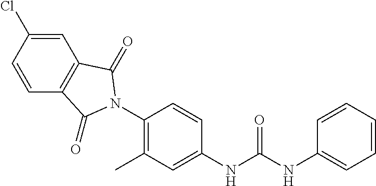

From 2-(4-amino-2-methylphenyl)-5-chloro-1,3-dihydroisoindole-1,3-dione and phenyl isocyanate in ethyl acetate at reflux.

LCMS (Method A) Rt 3.8 (M+H⁺) 406

### Intermediate 57

### N-[2-Chloro-4-(3-phenylureido)phenyl]acetamide

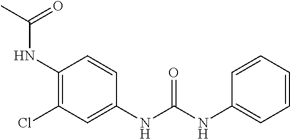

From N-(4-amino-2-chlorophenyl)acetamide and phenyl isocyanate in ethyl acetate at reflux

LCMS (Method B) Rt 3.03 (M+H⁺) 304

### Intermediate 58

### 1-(5-Nitropyridin-2-yl)-3-phenyl urea

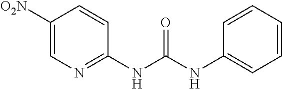

From 2-amino-5-nitropyridine and phenyl isocyanate in toluene at reflux

¹H NMR (400 MHz) (CDCl₃) δ 10.1 (s, 1H) 9.9 (s, 1H) 9.3 (s, 1H) 8.5 (d, 1H) 7.9 (d, 1H) 7.5 (d, 2H) 7.3 (t, 2H) 7.1 (t, 1H)

### Intermediate 59

### 1-(2-Methoxy-4-nitrophenyl)-3-phenyl urea

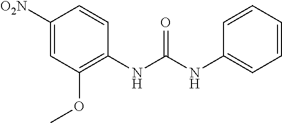

From 2-methoxy-4-nitrophenyl isocyanate and aniline in toluene at reflux

LCMS (Method A) Rt 3.78 (M+H⁺) 288

### Intermediate 60

### [N-(4-Aminophenyl)-N-(4′-fluorobiphenyl-3-sulphonyl)amino]acetic

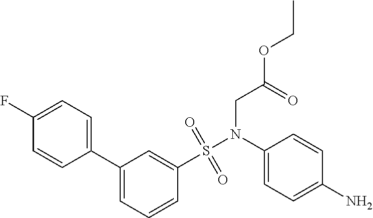

[N-(4′-Fluorobiphenyl-3-sulphonyl)-N-(4-nitrophenyl)amino]acetic acid ethyl ester (Intermediate 31, 450 mg) was dissolved in IMS (16 ml) and treated with palladium on carbon (10%, 65 mg). The reaction mixture was hydrogenated under a balloon of hydrogen at atmospheric pressure for 4 hours. The catalyst was removed by filtration through Celite under nitrogen and the filtrate was concentrated to give [N-(4-aminophenyl)-N-(4′-fluorobiphenyl-3-sulphonyl)amino]acetic acid ethyl ester (410 mg).

LCMS (Method A) Rt 3.74 (M+H⁺) 429

¹H NMR (300 MHz) (CDCl₃) δ 7.9 (s, 1H) 7.8 (dd, 1H) 7.7 (dd, 1H) 7.5 (m, 3H) 7.2 (t, 2H) 7.0 (d, 2H) 6.6 (d, 2H) 4.4 (s, 2H) 4.1 (q, 2H) 3.8 (br s, 2H) 1.2 (t, 3H)

By proceeding in a similar manner the following compounds were prepared from the appropriate starting materials:

### Intermediate 61

### 4′-Fluorobiphenyl-3-sulphonic acid N-(4-aminophenyl)-N-methylamide

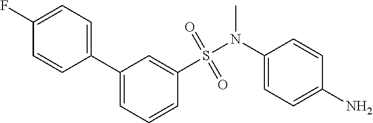

From 4′-fluorobiphenyl-3-sulphonic acid N-methyl-N-(4-nitrophenyl)amide (Intermediate 10)

LCMS (Method A) Rt 3.58 (M+H⁺) 357

### Intermediate 62

### N-(4-Aminophenyl)-N-methylcarbamic acid tert-butyl ester

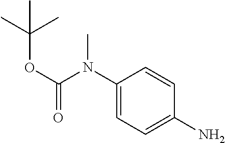

From N-methyl-N-(4-nitrophenyl)carbamic acid tert-butyl ester (Intermediate 20)

LCMS (Method A) Rt 2.38 (M+H⁺) 223

¹H NMR (400 MHz) (CDCl₃) δ 7.0 (d, 2H) 6.6 (d, 2H) 3.6 (br s, 2H) 3.2 (s, 3H) 1.5 (s, 9H)

### Intermediate 63

### 1-(3-Amino-4-methylphenyl)-3-phenyl urea

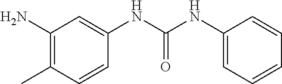

From 1-(4-methyl-3-nitrophenyl)-3-phenyl urea (Intermediate 46)

¹H NMR (400 MHz) δ (DMSO-d₆) δ 8.4 (s, 1H) 8.1 (s, 1H) 7.3 (m, 4H) 6.7 (m, 4H) 4.7 (br s, 2H) 1.9 (s, 3H)

### Intermediate 64

### 1-(3-Amino-4-methylphenyl)-3-benzyl urea

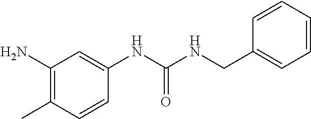

From 1-(4-methyl-3-nitrophenyl)-3-benzyl urea (Intermediate 50)

LCMS (Method A) Rt 2.24 (M+H⁺) 256

### Intermediate 65

### 1-(4-Aminophenyl)-3-(pyridin-4-yl)urea

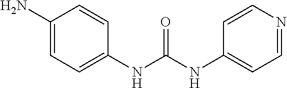

From 1-(4-nitrophenyl)-3-(pyridine-4-yl)urea (Intermediate 40)

¹H NMR (400 MHz) (DMSO-d₆) δ 8.9 (s, 1H) 8.3 (m, 3H) 7.4 (m, 2H) 7.2 (d, 2H) 6.5 (d, 2H) 5.8 (s, 2H)

### Intermediate 66

### 1-(5-Aminopyridin-2-yl)-3-phenyl urea

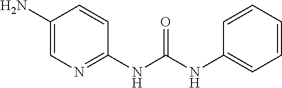

From 1-(5-nitropyridin-2-yl)-3-phenyl urea (Intermediate 58)

¹H NMR (400 MHz) (CDCl₃) δ 10.4 (br s, 1H) 9.0 (s, 1H) 7.7 (s, 1H) 7.5 (d, 2H) 7.3 (t, 2H) 7.2 (d, 1H) 7.0 (m, 2H) 5.0 (br s, 2H)

### Intermediate 67

### 1-(4-Aminophenyl)-3-[2-(pyridin-3-yl)ethyl]urea

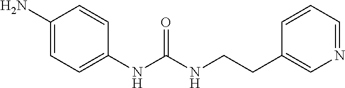

From 1-(4-nitrophenyl)-3-[2-(pyridin-3-yl)ethyl]urea (Intermediate 51)

The compound used without purification or characterisation

### Intermediate 68

### 1-(4-Amino-3-methylphenyl)-3-(2-methylphenyl)urea

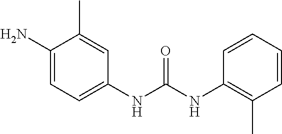

From 1-(3-methyl-4-nitrophenyl)-3-(2-methylphenyl)urea (Intermediate 52)

LCMS (Method A) Rt 2.37 (M+H⁺) 256

### Intermediate 69

### 6-Amino-2,3-dihydroindole-1-carboxylic acid N-phenylamide

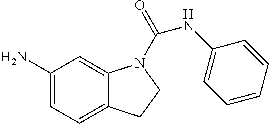

From 6-nitro-2,3-dihydroindole-1-carboxylic acid N-phenylamide (Intermediate 53)

LCMS (Method A) Rt 1.83 & 2.01 (M+H⁺) 254

### Intermediate 70

### 3-(2-Methoxyethoxy)aniline

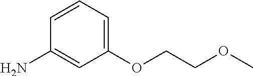

From 3-(2-methoxyethoxy)nitrobenzene (Intermediate 30)

LCMS (Method A) Rt 0.37 & 1.55 (M+H⁺) 168

¹H NMR (400 MHz) (CDCl₃) δ 7.1 (t, 1H) 6.3 (m, 3H) 4.1 (t, 2H) 3.7 (t, 2H) 3.6 (br s, 2H) 3.4 (s, 3H)

### Intermediate 71

### 1-(4-Aminophenyl)-3-[3-(2-methoxyethoxy)phenyl]urea

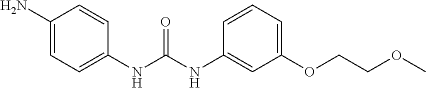

From 1-(4-nitrophenyl)-3-[3-(2-methoxyethoxy)phenyl]urea (Intermediate 42)

LCMS (Method A) Rt 0.37 & 2.09 (M+H⁺) 302

¹H NMR (400 MHz) (DMSO-d₆) δ 8.6 (br s 1H) 8.2 (br s, 1H) 7.2-7.0 (m, 4H) 6.9 (d, 1H) 6.5 (m, 3H) 4.8 (br s, 2H) 4.0 (t, 2H) 3.6 (t, 2H) 3.3 (s, 3H)

### Intermediate 72

### 1-(4-Amino-2-methoxyphenyl)-3-phenyl urea

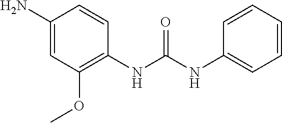

From 1-(2-methoxy-4-nitrophenyl)-3-phenyl urea (Intermediate 59)

¹H NMR (400 MHz) (DMSO-d₆) δ 9.0 (s, 1H) 7.7 (s, 1H) 7.6 (d, 1H) 7.4 (d, 2H) 7.25 (t, 2H) 6.9 (t, 1H) 6.3 (d, 1H) 6.1 (dd, 1H) 4.8 (s, 2H) 3.8 (s, 3H)

### Intermediate 73

### 1-(5-Amino-2-methoxyphenyl)-3-phenyl urea

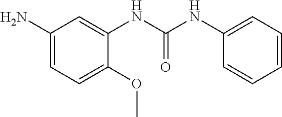

From 1-(2-methoxy-5-nitrophenyl)-3-phenyl urea (Intermediate 55)

LCMS (Method A) Rt 2.06 (M+H⁺) 258

### Intermediate 74

### 4′-Fluorobiphenyl-3-sulphonic acid N-(5-amino-2-methoxyphenyl)amide

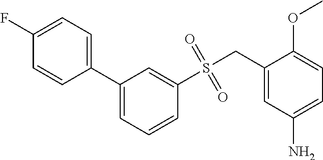

From 4′-fluorobiphenyl-3-sulphonic acid N-(2-methoxy-5-nitrophenyl)amide (Intermediate 5)

LCMS (Method A) Rt 2.68 (M+H⁺) 373

### Intermediate 75

### 4′-Fluorobiphenyl-3-sulphonic acid N-(4-amino-2-methoxyphenyl)-N-(3-tert-butoxypropyl)-amide

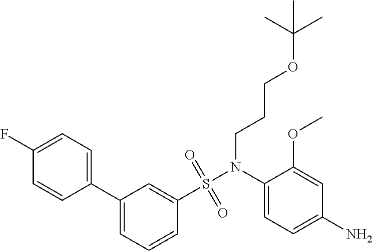

From 4′-fluorobiphenyl-3-sulphonic acid N-(2-methoxy-4-nitrophenyl)-N-(3-tert-butoxy-propyl)amide (Intermediate 29)

¹H NMR (400 MHz) (DMSO-d₆) δ 7.9 (dt, 1H) 7.70-7.55 (m, 5H) 7.3 (t, 2H) 6.7 (d, 1H) 6.1 (m, 2H) 5.3 (br s, 2H) 3.5 (t, 2H) 3.3 (s, 3H) 3.25 (t, 2H) 1.5 (m, 2H) 1.1 (s, 9H)

### Intermediate 76

### 4′-Fluorobiphenyl-3-sulphonic acid (4-amino-2-methoxyphenyl)amide

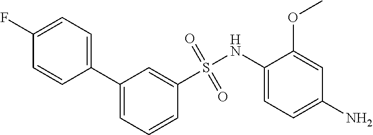

From 4′-fluorobiphenyl-3-sulphonic acid (2-methoxy-4-nitrophenyl)amide (Intermediate 13)

¹H NMR (400 MHz) (CDCl₃) δ 7.8 (s, 1H) 7.6 (d, 2H) 7.4 (m, 3H) 7.35 (d, 1H) 7.1 (t, 2H) 6.5 (br s, 1H) 6.25 (dd, 1H) 6.0 (s, 1H) 3.65 (br s, 2H) 3.35 (s, 3H)

### Intermediate 77

### 4′-Fluorobiphenyl-3-sulphonic acid N-(4-amino-2-methylphenyl)-N-methylamide

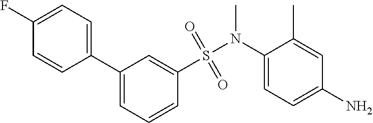

From 4′-fluorbiphenyl-3-sulphonic acid N-(2-methyl-4-nitrophenyl)-N-methylamide (Intermediate 32)

LCMS (Method A) Rt 3.56 (M+H⁺) 371

### Intermediate 78

### 4′-Fluorobiphenyl-3-sulphonic acid N-(4-aminophenyl)-N-[(2,2-dimethyl-1,3-dioxolan-4-yl)methyl]amide

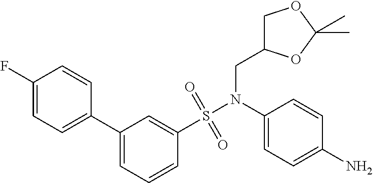

From 4′-fluorobiphenyl-3-sulphonic acid N-(4-nitrophenyl)-N-[(2,2-dimethyl-1,3-dioxolan-4-yl)methyl]amide (Intermediate 18)

LCMS (Method B) Rt 3.83 (M+H⁺) 457

### Intermediate 79

### 4-Aminophenylsulphonic acid (4′-fluorobiphenyl-3-yl)amide

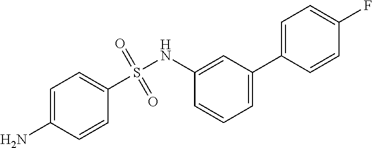

From 4-nitrophenylsulphonic acid (4′-fluorobiphenyl-3-yl)amide (Intermediate 34)

LCMS (Method A) Rt 3.5 (M+H⁺) 343

### Intermediate 80

### 1-(4-Aminophenyl)-3-(1-phenylethyl)urea

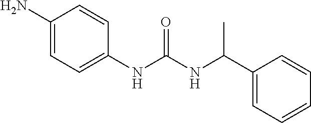

To a solution of 1-(4-nitrophenyl)-3-(1-phenylethyl)urea (Intermediate 54, 583 mg) in ethanol (50 ml) was added SnCl₂.2H₂O (4.27 g). The mixture was stirred and heated at 70° C. overnight. The reaction mixture was cooled to room temperature, poured onto a mixture of ice and water and then diluted with aqueous NaOH. The mixture was extracted with ethyl acetate and the organic layer was dried (MgSO₄) and filtered. The volatiles were removed by evaporation to give 1-(4-aminophenyl)-3-(1-phenylethyl)urea (156 mg).

LCMS (Method A) Rt 2.06 (M+H⁺) 256

By proceeding in a similar manner the following compounds were prepared from the appropriate starting materials:

### Intermediate 81

### 1-(5-Amino-2-chlorophenyl)-3-phenyl urea

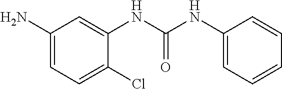

From 1-(2-chloro-5-nitrophenyl)-3-phenyl urea (Intermediate 47)

LCMS (Method A) Rt 2.75 (M+H⁺) 262

### Intermediate 82

### 1-(3-Amino-4-chlorophenyl)-3-phenyl urea

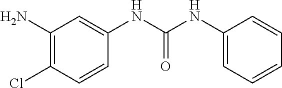

From 1-(4-chloro-3-nitrophenyl)-3-phenyl urea (Intermediate 48)

LCMS (Method A) Rt 3.23 (M+H⁺) 2.62

### Intermediate 83

### 1-(4-Amino-2-chlorophenyl)-3-phenyl urea

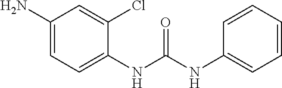

From 1-(2-chloro-4-nitrophenyl)-3-phenyl urea (Intermediate 49)

¹H NMR (400 MHz) (DMSO-d₆) □ 8.9 (s, 1H) 7.8 (s, 1H) 7.5 (d, 1H) 7.4 (d, 2H) 7.25 (t, 2H) 6.9 (t, 1H) 6.65 (d, 1H) 6.5 (dd, 1H) 5.2 (s, 2H)

### Intermediate 84

### 4′-Fluorobiphenyl-3-sulphonic acid N-(4-aminophenyl)-N-(3-benzyloxypropyl)amide

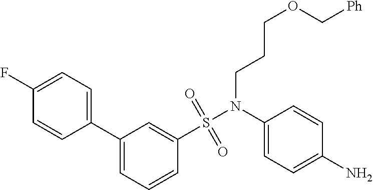

From 4′-fluorobiphenyl-3-sulphonic acid N-(4-nitrophenyl)-N-(3-benzyloxypropyl)amide (Intermediate 28)

LCMS (Method B) Rt 4.23 (M+H⁺) 491

### Intermediate 85

### 1-(4-Amino-3-chlorophenyl)-3-phenyl urea

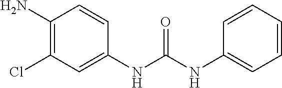

To a suspension of N-[2-chloro-4-(3-phenylureido)phenyl]acetamide (Intermediate 57, 690 mg) in ethanol (10 ml) was added HCl (37%, 10 ml). The reaction mixture was heated at reflux for 2 hours, then cooled and poured directly onto a SCX-2 column. It was eluted with DCM, then MeOH and then with a mixture DCM and 2M ammonia in MeOH (9:1) to give 1-(4-amino-3-chlorophenyl)-3-phenyl urea as a yellow solid (250 mg).

LCMS (Method B) Rt 2.92 (M+H⁺) 262

### Intermediate 86

### 1-(4-Fluorophenyl)-1H-pyrazole

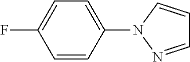

To a solution of 4-fluorophenyl boronic acid (750 mg) in pyridine (67 ml) was added copper acetate (1.95 g) and pyrazole (729 mg). The mixture was stirred in an open reaction vessel at 40° C. overnight. The pyridine was removed by evaporation and the residue was partitioned between water and ethyl acetate. The organic layer was washed with water, dried (Na₂SO₄) and filtered. The volatiles were removed by evaporation to give 1-(4-fluorophenyl)-1H-pyrazole (816 mg).

LCMS (Method A) Rt 3.18 (M+H⁺) 163

¹H NMR (300 MHz) (DMSO-d₆) δ 8.5 (s, 1H) 7.9 (m, 2H) 7.7 (s, 1H) 7.3 (m, 2H) 6.5 (s, 1H)

### Intermediate 87

### 1-(4-Fluorophenyl)-1H-pyrazole-4-sulphonyl chloride

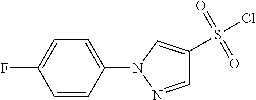

1-(4-Fluorophenyl)-1H-pyrazole (Intermediate 86, 300 mg) was dissolved in chloroform (24 ml) and chlorosulphonic acid (1.23 ml) was added. The mixture was heated under reflux for 3 hours then the volatiles were removed by evaporation. The residue was treated with thionyl chloride (9.2 ml) and DMF (9 drops) and the mixture was stirred and heated at 100° C. for 2 hours, then cooled to room temperature. The volatiles were again removed by evaporation and the residue was treated with toluene and re-evaporated. The residue was then partitioned between water and ethyl acetate and the organic layer was dried (Na₂SO₄) and filtered. The volatiles were removed by evaporation to give 1-(4-fluorophenyl)-1H-pyrazole-4-sulphonyl chloride as a pale brown oil (480 mg).

¹H NMR (300 MHz) (DMSO-d₆) δ 8.5 (s, 1H) 7.9 (m, 2H) 7.7 (s, 1H) 7.3 (t, 2H)

By proceeding in a similar manner the following compounds were prepared from the appropriate starting materials:

### Intermediate 88

### 3,5-Dimethyl-1-(pyridin-2-yl)-1H-pyrazole-4-sulphonyl chloride

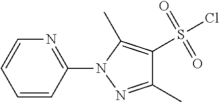

From 3,5-dimethyl-1-(pyridin-2-yl)-1H-pyrazole

LCMS (Method A) Rt 3.82 (M+H⁺) 272

¹H NMR (300 MHz) (DMSO-d₆) δ 8.5 (dd, 1H) 8.0 (m, 1H) 7.7 (d, 1H) 7.4 (m, 1H), 2.7 (s, 3H) 2.3 (s, 3H)

### Intermediate 89

### 1-(Pyridin-2-yl)-1H-pyrazole-4-sulphonyl chloride

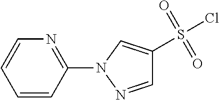

From 1-(pyridine-2-yl)-1H-pyrazole (Intermediate 93)

LCMS (Method A) Rt 3.64 (M+H⁺) 244

### Intermediate 90

### 1-(5-Fluoropyridin-2-yl)-1H-pyrazole-4-sulphonyl chloride

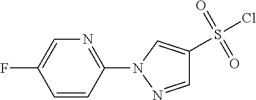

From 1-(5-fluoropyridin-2-yl)-1H-pyrazole (Intermediate 92)

¹H NMR (300 MHz) (DMSO-d₆) δ 8.5 (s, 1H) 8.3 (s, 1H) 7.9 (m, 2H) 7.6 (s, 1H)

### Intermediate 91

### 2-(4-Fluorophenyl)-1-methyl-1H-imidazole-4-sulphonyl chloride

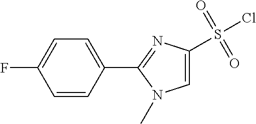

From 2-(4-fluorophenyl)-1-methyl-1H-imidazole

LCMS (Method B) Rt 3.46 (M+H⁺) 275

¹H NMR (400 MHz) (DMSO-d₆) δ 7.75 (s, 1H) 7.65 (m, 2H) 7.20 (m, 2H) 3.9 (s, 3H)

### Intermediate 92

### 1-(5-Fluoropyrid-2-yl)-1H-pyrazole

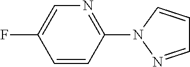

A suspension of copper (I) iodide (48 mg), L-proline (59 mg), potassium carbonate (730 mg), 2-bromo-5-fluoropyridine (500 mg) and pyrazole (175 mg) in DMSO (3.3 ml) was heated in the microwave for 2 hours at 140° C. The resultant mixture was partitioned between water and ethyl acetate and the organic layer was dried (MgSO₄) and filtered. The volatiles were removed by evaporation and the residue was purified by chromatography on a 5 g silica cartridge eluting with initially cyclohexane increasing the polarity to a mixture of ethyl acetate and cyclohexane (1:20) to give 1-(5-fluoropyrid-2-yl)-1H-pyrazole (200 mg).

LCMS (Method B) Rt 2.94 (M+H⁺) 164

¹H NMR (300 MHz) (DMSO-d₆) δ 8.6 (d, 1H) 8.5 (t, 1H) 8.0 (m, 2H) 7.8 (d, 1H) 6.6 (dd, 1H)

By proceeding in a similar manner the following compound was prepared from the appropriate starting materials:

### Intermediate 93

### 1-(Pyrid-2-yl)-1H-pyrazole

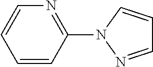

From 2-bromopyridine and pyrazole

¹H NMR (300 MHz) (DMSO-d₆) δ 8.6 (s, 1H) 8.5 (m, 1H) 8.0 (m, 2H) 7.8 (s, 1H) 7.3 (t, 1H) 6.6 (s, 1H)

### Example 1

### 4′-Fluorobiphenyl-3-sulphonic acid {4-[3-(4-methoxyphenyl)ureido]-phenyl}amide

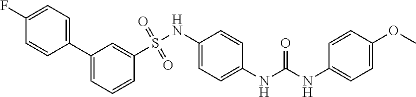

4′-Fluorobiphenyl-3-sulphonic acid (4-aminophenyl)amide (Intermediate 22, 50 mg) was dissolved in THF (2 ml) and treated with NaOH (1M, 300 μl) and 4-methoxyphenyl isocyanate (29 μl). After stirring at room temperature for 20 hours, the THF was removed by evaporation and the residue was acidified to pH 5 and extracted with ethyl acetate. The organic layer was dried (Na₂SO₄), filtered and the volatiles were removed by evaporation. The residue was purified by HPLC eluting with a mixture of water and acetonitrile (each containing 0.1% of formic acid) from 20 to 98% acetonitrile over 30 minutes to give 4′-fluorobiphenyl-3-sulphonic acid {4-[3-(4-methoxyphenyl)ureido]-phenyl}amide as a white solid (62 mg).

LCMS (Method C) Rt 11.30 (M+H⁺) 492

¹H NMR (400 MHz) (DMSO-d₆) δ 10.0 (br s, 1H) 8.5 (br s, 1H) 8.4 (br s, 1H) 7.9 (m, 2H) 7.7 (m, 4H) 7.3 (m, 6H) 7.0 (d, 2H) 6.8 (d, 2H) 3.7 (s, 3H)

By proceeding in a similar manner the following compounds were prepared from the appropriate starting materials:

### Example 2

### 4′-Fluorobiphenyl-3-sulphonic acid {4-[3-(2-chlorophenyl)ureido]-phenyl}amide

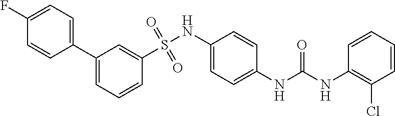

From 4′-fluorobiphenyl-3-sulphonic acid (4-aminophenyl)amide (Intermediate 22) and 2-chlorophenyl isocyanate

LCMS (Method C) Rt 12.34 (M+H⁺) 496

¹H NMR (400 MHz) (DMSO-d₆) δ 10.0 (br s, 1H) 9.3 (s, 1H) 8.3 (s, 1H) 8.1 (d, 1H) 7.9 (m, 2H) 7.7 (m, 4H) 7.4 (d, 1H) 7.3 (m, 5H) 7.0 (m, 3H)

### Example 3

### 4′-Fluorobiphenyl-3-sulphonic acid {4-[3-(2-methoxyphenyl)ureido]-phenyl}amide

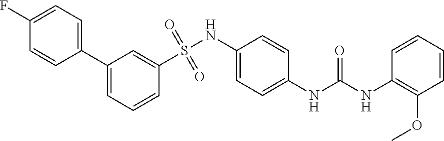

From 4′-fluorobiphenyl-3-sulphonic acid (4-aminophenyl)amide (Intermediate 22) and 2-methoxyphenyl isocyanate

LCMS (Method C) Rt 11.94 (M+H⁺) 492

¹H NMR (400 MHz) (DMSO-d₆) δ 10.0 (br s, 1H) 9.2 (s, 1H) 8.2 (s, 1H) 8.1 (d, 1H) 7.9 (m, 2H) 7.7 (m, 4H) 7.3 (m, 4H) 7.0 (m, 3H) 6.9 (m, 2H) 3.9 (s, 3H)

### Example 4

### 4′-Fluorobiphenyl-3-sulphonic acid {4-[3-(2-methylphenyl)ureido]-phenyl}amide

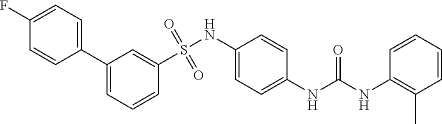

4′-Fluorobiphenyl-3-sulphonic acid (4-aminophenyl)amide (Intermediate 22, 50 mg) was dissolved in ethyl acetate (2.5 ml) and then treated with 2-methylphenyl isocyanate (22 μl). The resultant mixture was stirred and heated at 85° C. overnight. The reaction mixture was cooled to room temperature and partitioned between aqueous citric acid (10%) and ethyl acetate. The organic layer was dried (Na₂SO₄), filtered and the volatiles were removed by evaporation. The residue was purified by HPLC eluting with a mixture of water and acetonitrile (each containing 0.1% formic acid) from 30 to 98% acetonitrile over 20 minutes to give 4′-fluorobiphenyl-3-sulphonic acid {4-[3-(2-methylphenyl)ureido]phenyl}amide as a white solid (15 mg).

LCMS (Method C) Rt 11.78 (M+H⁺) 476

¹H NMR (400 MHz) (CDCl₃) δ 7.85 (s, 1H) 7.7 (m, 2H) 7.5-7.4 (m, 4H) 7.3-7.2 (m, 5H) 7.1 (m, 2H) 7.0 (m, 2H) 6.3 (br s, 2H) 6.0 (s, 1H) 2.3 (s, 3H)

By proceeding in a similar manner the following compounds were prepared from the appropriate starting materials. Alternative solvents such as THF or toluene may be used and the reaction may be carried out at room temperature or at reflux.

### Example 5

### 4′-Fluorobiphenyl-3-sulphonic acid [2-methoxy-5-(3-phenylureido)phenyl]amide

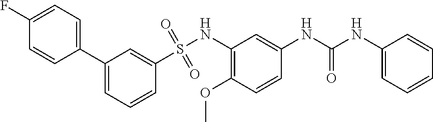

From 4′-fluorobiphenyl-3-sulphonic acid (5-amino-2-methoxyphenyl)amide (Intermediate 74) and phenyl isocyanate.

LCMS (Method C) Rt 11.78 (M+H⁺) 492

¹H NMR (400 MHz) (DMSO-d₆) δ 9.5 (s, 1H) 8.6 (s, 1H) 8.5 (s, 1H) 8.0 (s, 1H) 7.9 (d, 1H) 7.7 (m, 3H) 7.6 (t, 1H) 7.5 (s, 1H) 7.4 (d, 2H) 7.3 (m, 4H) 7.1 (dd, 1H) 6.9 (t, 1H) 6.8 (d, 1H) 3.5 (s, 3H)

### Example 6

### 4′-Fluorobiphenyl-3-sulphonic acid {4-[3-(4-cyanophenyl)ureido]-phenyl}amide

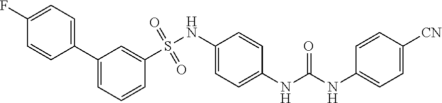

From 4′-fluorobiphenyl-3-sulphonic acid (4-aminophenyl)amide (Intermediate 22) and 4-cyanophenyl isocyanate in THF at reflux

LCMS (Method C) Rt 11.36 (M+H⁺) 487

¹H NMR (400 MHz) (DMSO-d₆) δ 10.1 (s, 1H) 9.2 (s, 1H) 8.8 (s, 1H) 7.9 (m, 2H) 7.7-7.6 (m, 8H) 7.3 (m, 4H) 7.0 (d, 2H)

### Example 7

### 4′-Fluorobiphenyl-3-sulphonic acid N-methyl-N-{4-[3-(4-pyridyl)ureido]phenyl}amide

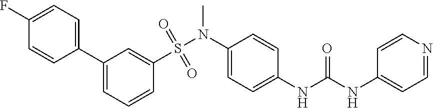

From 4′-fluorobiphenyl-3-sulphonic acid N-(4-aminophenyl)-N-methylamide (Intermediate 61) and 4-pyridyl isocyanate in ethyl acetate at room temperature

LCMS (Method C) Rt 7.84 (M+H⁺) 477

¹H NMR (400 MHz) (CD₃OD) δ 8.4 (br s, 2H) 7.9 (d, 1H) 7.7 (m, 2H) 7.6 (m, 2H) 7.5 (m, 3H) 7.4 (d, 2H) 7.2 (t, 2H) 7.1 (d, 2H) 3.2 (s, 3H)

### Example 8

### 4′-Fluorobiphenyl-3-sulphonic acid N-methyl-N-[2-methyl-4-(3-phenylureido)phenyl]amide

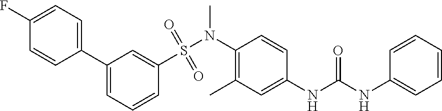

From 4′-fluorobiphenyl-3-sulphonic acid N-(4-amino-2-methylphenyl)-N-methylamide (Intermediate 77) and phenyl isocyanate in ethyl acetate at room temperature

LCMS (Method C) Rt 12.57 (M+H⁺) 490

¹H NMR (400 MHz) (DMSO-d₆) δ 8.6 (s, 1H) 8.5 (s, 1H) 8.0 (m, 1H) 7.8 (t, 3H) 7.7 (m, 2H) 7.3 (d, 2H) 7.2 (m, 6H) 7.0 (s, 1H) 6.9 (t, 1H) 3.1 (s, 3H) 2.2 (s, 3H)

### Example 9

### 4′-Fluorobiphenyl-3-sulphonic acid N-methyl-N-[2-methoxy-4-(3-phenylureido)phenyl]amide

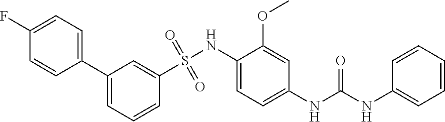

From 4′-fluorobiphenyl-3-sulphonic acid (4-amino-2-methoxyphenyl)amide (Intermediate 76) and phenyl isocyanate in toluene at reflux.

LCMS (Method C) Rt 11.70 (M+H⁺) 492

¹H NMR (400 MHz) (DMSO-d₆) δ 9.4 (br s, 1H) 8.8 (s, 1H) 8.7 (s, 1H) 7.9 (m, 2H) 7.7 (m, 4H) 7.4 (d, 2H) 7.3 (m, 4H) 7.2 (s, 1H) 7.1 (d, 1H) 7.0 (t, 1H) 6.9 (d, 1H) 3.3 (s, 3H)

### Example 10

### 4-(3-Phenylureido)phenylsulphonic acid (4′-fluorobiphenyl-3-yl)amide

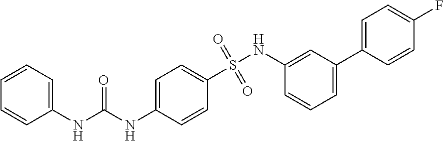

From 4-aminophenylsulphonic acid (4′-fluorobiphenyl-3-yl)amide (Intermediate 79) and phenyl isocyanate in toluene at reflux.

LCMS (Method C) Rt 11.81 (M+H⁺) 462

¹H NMR (400 MHz) (DMSO-d₆) δ 10.2 (br s, 1H) 9.0 (s, 1H) 8.7 (s, 1H) 7.7 (d, 2H) 7.6-7.5 (m, 4H) 7.4 (br d, 2H) 7.3-7.2 (m, 7H) 7.0 (dt, 1H) 6.9 (br t, 1H).

### Example 11

### 2,4-Dichlorophenyl sulphonic acid N-methyl-N-[4-(3-phenylureido)phenyl]amide

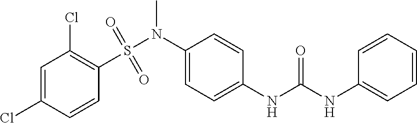

To a solution of 2,4-dichlorophenylsulphonyl chloride (60 mg) in pyridine (1.5 ml) was added 1-(4-methylaminophenyl)-3-phenyl urea (Intermediate 25, 59 mg). The reaction mixture was stirred for 4 hours at room ternperature and then the volatiles were removed by evaporation. The residue was partitioned between saturated aqueous NaHCO₃ and ethyl acetate. The organic layer was dried (Na₂SO₄), filtered and volatiles were removed by evaporation. The residue was purified by HPLC eluting with a mixture of water and acetonitrile (each containing 0.1% formic acid) from 20 to 98% over 25 minutes to give 2,4-dichlorophenyl sulphonic acid N-methyl-N-[4-(3-phenylureido)phenyl]amide (40 mg).

LCMS (Method C) Rt 12.08 (M+H⁺) 450

¹H NMR (400 MHz) (DMSO-d₆) δ 8.8 (br s, 1H) 8.7 (br s, 1H) 7.9 (s, 1H) 7.8 (d, 1H) 7.6 (d, 1H) 7.4 (m, 4H) 7.3 (t, 2H) 7.1 (d, 2H) 7.0 (t, 1H) 3.3 (s, 3H)

By proceeding in a similar manner the following compounds were prepared from the appropriate starting materials. The reactions may be performed using alternative solvents such as DCM or NMP in the presence of a base such as pyridine or N,N-diisopropyl-N-ethylamine.

### Example 12

### 4′-Fluorobiphenyl-3-sulphonic acid N-methyl-N-[4-(3-phenylureido)phenyl]amide

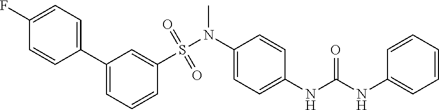

From 4′-fluorobiphenyl-3-sulphonyl chloride and 1-(4-methylaminophenyl)-3-phenyl urea (Intermediate 25)

LCMS (Method C) Rt 12.47 (M+H⁺) 476

¹H NMR (400 MHz) (DMSO-d₆) δ 8.8 (s, 1H) 8.7 (s, 1H) 8.0 (d, 1H) 7.7 (m, 3H) 7.6 (s, 1H) 7.5 (d, 1H) 7.4 (m, 4H) 7.3 (m, 4H) 7.0 (m, 3H) 3.2 (s, 3H)

### Example 13

### 3-Difluoromethoxyphenylsulphonic acid N-methyl-N-[4-(3-phenylureido)phenyl]amide

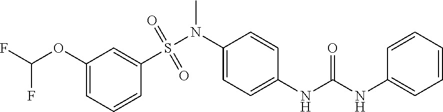

From 3-difluoromethoxyphenylsulphonyl chloride and 1-(4-methylaminophenyl)-3-phenyl urea (Intermediate 25)

LCMS (Method C) Rt 11.35 (M+H⁺) 448

¹H NMR (400 MHz) (DMSO-d₆) δ 8.9 (s, 1H) 8.7 (s, 1H) 7.7 (t, 1H) 7.5 (dd, 1H) 7.4 (m, 5H) 7.3 (m, 4H) 7.0 (m, 3H) 3.1 (s, 3H)

### Example 14

### 2-Chloro-4-trifluoromethylphenyl sulphonic acid N-methyl-N-[4-(3-phenylureido)phenyl]amide

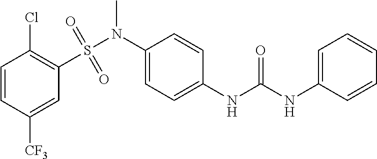

From 2-chloro-5-trifluoromethylphenylsuphonyl chloride and 1-(4-methylaminophenyl)-3-phenyl urea (Intermediate 25)

LCMS (Method C) Rt 12.11 (M+H⁺) 484

¹H NMR (400 MHz) (DMSO-d₆) δ 8.8 (s, 1H) 8.7 (s, 1H) 8.1 (d, 1H) 8.0 (d, 1H) 7.9 (s, 1H) 7.4 (m, 4H) 7.3 (t, 2H) 7.1 (d, 2H) 7.0 (t, 1H) 3.3 (s, 3H)

### Example 15

### 4′-Fluorobiphenyl-3-sulphonic acid {4-[3-(pyridin-4-yl)ureido]phenyl}-amide

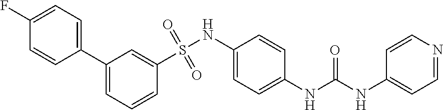

From 4′ fluorobiphenylsulphonyl chloride and 1-(4-aminophenyl)-3-(pyrid-4-yl)urea (Intermediate 65)

LCMS (Method C) Rt 7.42 (M+H⁺) 463

¹H NMR (400 MHz) (DMSO-d₆) δ 10.0 (br s, 1H) 9.0 (s, 1H) 8.8 (s, 1H) 8.3 (d, 2H) 7.9 (m, 2H) 7.7 (m, 4H) 7.3 (m, 6H) 7.0 (d, 2H)

### Example 16

### 4′-Fluorobiphenyl-3-sulphonic acid {4-[3-(2{pyridin-3-yl}ethyl]ureido}-phenyl)amide

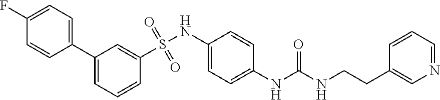

From 4′-fluorobiphenyl-3-sulphonyl chloride and 1-(4-aminophenyl)-3-[2-(pyridin-3-yl)ethyl]urea (Intermediate 67)

LCMS (Method C) Rt 7.26 (M+H⁺) 491

¹H NMR (400 MHz) (DMSO-d₆) δ 9.9 (s, 1H) 8.4 (m, 3H) 7.9 (m, 2H) 7.7 (m, 5H) 7.3 (m, 3H) 7.2 (d, 2H) 6.9 (d, 2H) 6.1 (t, 1H) 3.3 (m, 2H) 2.7 (t, 2H)

### Example 17

### 4′-Fluorobiphenyl-3-sulphonic acid [2-chloro-5-(3-phenylureido)phenyl]amide

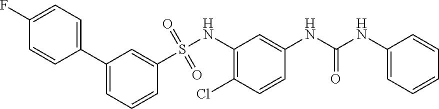

From 4′-fluorobiphenyl-3-sulphonyl chloride and 1-(3-Amino-4-chlorophenyl)-3-phenyl urea (Intermediate 82)

LCMS (Method C) Rt 12.38 (M+H⁺) 496

¹H NMR (400 MHz) (DMSO-d₆) δ 10.0 (s, 1H) 8.9 (s, 1H) 8.6 (s, 1H) 8.0 (s, 1H) 7.9 (d, 1H) 7.8 (d, 1H) 7.7 (m, 4H) 7.4 (d, 2H) 7.3 (m, 5H) 7.2 (d, 1H) 7.0 (t, 1H)

### Example 18

### 4′-Fluorobiphenyl-3-sulphonic acid [5-(3-benzylureido)-2-methylphenyl]amide

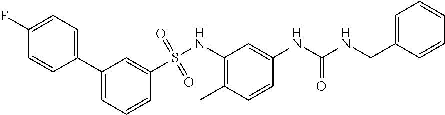

From 4′-fluorobiphenyl-3-sulphonyl chloride and 1-(3-amino-4-methylphenyl)-3-benzyl urea (Intermediate 64)

LCMS (Method C) Rt 11.69 (M+H⁺) 490

¹H NMR (400 MHz) (DMSO-d₆) δ 9.5 (s, 1H) 8.5 (s, 1H) 7.9 (m, 2H) 7.7 (m, 4H) 7.3 (m, 8H) 7.2 (d, 1H) 6.9 (d, 1H) 6.5 (t, 1H) 4.3 (d, 2H) 1.9 (s, 3H)

### Example 19

### 4′-Fluorobiphenyl-3-sulphonic acid {2-methyl-4-[3-(2-methylphenyl)ureido]phenyl}amide

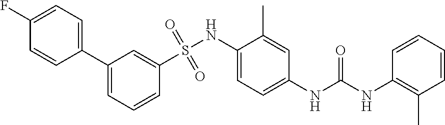

From 4′-fluorobiphenyl-3-sulphonyl chloride and 1-(4-amino-3-methylphenyl)-3-(2-methylphenyl)urea (Intermediate 68)

LCMS (Method C) Rt 12.14 (M+H⁺) 490

¹H NMR (400 MHz) (DMSO-d₆) δ 9.6 (br s, 1H) 9.0 (s, 1H) 7.9 (m, 2H) 7.8 (m, 2H) 7.7 (m, 4H) 7.3 (m, 3H) 7.2 (m, 3H) 7.0 (d, 1H) 6.9 (t, 1H) 2.2 (s, 3H) 1.9 (s, 3H)

### Example 20

### 6-(4′-Fluorobiphenyl-3-sulphonylamino)-2,3-dihydroindole-1-carboxylic acid N-phenylamide

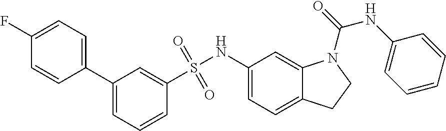

From 4′fluorobiphenyl-3-sulphonyl chloride and 6-amino-2,3-dihydroindole-1-carboxylic acid N-phenylamide (Intermediate 69)

LCMS (Method C) Rt 11.98 (M+H⁺) 488

¹H NMR (400 MHz) (DMSO-d₆) δ 10.2 (br s, 1H) 8.4 (s, 1H) 8.0 (s, 1H) 7.9 (m, 2H) 7.7 (m, 3H) 7.6 (t, 1H) 7.5 (d, 2H) 7.3 (t, 2H) 7.2 (t, 2H) 7.0 (m, 2H) 6.7 (d, 1H) 4.1 (t, 2H) 3.1 (t, 2H)

### Example 21

### 4′-Fluorobiphenyl-3-sulphonic acid [2-methyl-4-(3-phenylureido)phenyl]amide

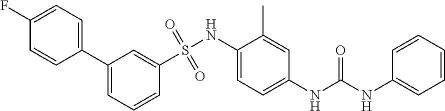

From 4′-fluorobiphenyl-3-sulphonyl chloride and 1-(4-amino-3-methylphenyl)-3-phenyl urea (Intermediate 27)

LCMS (Method C) Rt 11.45 (M+H⁺) 476

¹H NMR (400 MHz) (DMSO-d₆) δ 9.4 (s, 1H) 8.65 (s, 1H) 8.6 (s, 1H) 7.9 (dd, 1H) 7.8 (s, 1H) 7.6 (m, 4H) 7.4 (d, 2H) 7.3 (m, 5H) 7.2 (dd, 1H) 7.0 (t, 1H) 6.8 (d, 1H) 2.0 (d, 3H)

### Example 22

### 4′-Fluorobiphenyl-3-sulphonic acid {4-[3-(1-phenylethyl)ureido]phenyl}amide

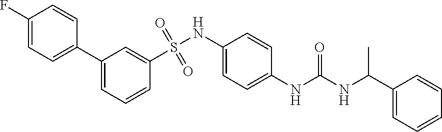

From 4′-fluorobiphenylsulphonyl chloride and 1-(4-aminophenyl)-3-(1-phenylethyl)urea (Intermediate 80)

LCMS (Method C) Rt 11.55 (M+H⁺) 490

¹H NMR (400 MHz) (DMSO-d₆) δ 9.9 (br s, 1H) 8.3 (s, 1H) 7.9 (m, 2H) 7.6 (m, 4H) 7.3 (m, 6H) 7.2 (m, 3H) 6.9 (d, 2H) 6.6 (d, 1H) 4.8 (dq, 1H) 1.4 (d, 3H)

### Example 23

### 4′-Fluorobiphenyl-3-sulphonic acid (4-{3-[3-(2-methoxyethoxy)phenyl]ureido}phenyl)amide

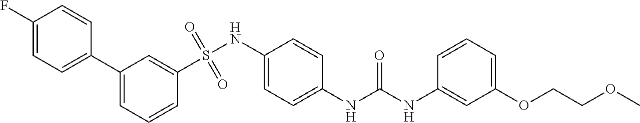

From 4′-fluorobiphenylsulphonyl chloride and 1-(4-aminophenyl)-3-[3-(2-methyoxy-ethoxy)phenyl]urea (Intermediate 71)

LCMS (Method C) Rt 11.32 (M+H⁺) 536

¹H NMR (400 MHz) (DMSO-d₆) δ 10.0 (s, 1H) 8.6 (s, 1H) 8.55 (s, 1H) 7.9 (m, 2H) 7.7 (m, 4H) 7.3 (m, 4H) 7.1 (m, 2H) 7.0 (d, 2H) 6.9 (d, 1H) 6.5 (d, 1H) 4.0 (t, 2H) 3.6 (t, 2H) 3.3 (s, 3H)

### Example 24

### 1-(4-Fluorophenyl)-3,5-dimethyl-1H-pyrazole-4-sulphonic acid [4-(3-phenylureido)phenyl]-amide

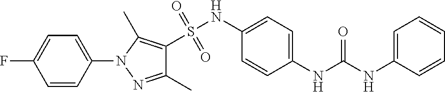

From 1-(4-fluorophenyl)-3,5-dimethyl-1H-pyrazole-4-sulphonyl chloride and 1-(4-amino-phenyl)-3-phenyl urea (Intermediate 23)

LCMS (Method C) Rt 10.52 (M+H⁺) 480

¹H NMR (400 MHz) (DMSO-d₆) δ 9.8 (s, 1H) 8.6 (s, 2H) 7.5 (m, 2H) 7.4 (d, 2H) 7.3 (m, 4H) 7.2 (t, 2H) 7.0 (m, 3H) 2.2 (s, 6H)

### Example 25

### 1-(4-Fluorophenyl)-3,5-dimethyl-1H-pyrazole-4-sulphonic acid N-methyl-N-[4-(3-phenyl-ureido)phenyl]amide

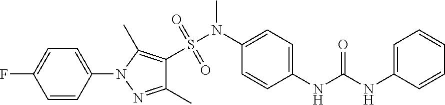

From 1-(4-fluorophenyl)-3,5-dimethyl-1H-pyrazole-4-sulphonyl chloride and 1-(4-methyl-aminophenyl)-3-phenyl urea (Intermediate 25)

LCMS (Method C) Rt 11.44 (M+H⁺) 494

¹H NMR (400 MHz) (DMSO-d₆) δ 8.8 (s, 1H) 8.7 (s, 1H) 7.6 (m, 2H) 7.4 (m, 6H) 7.3 (t, 2H) 7.2 (d, 2H) 7.0 (t, 1H) 3.1 (s, 3H) 2.1 (s, 3H) 2.0 (s, 3H)

### Example 26

### 5-(1-Methyl-3-trifluoromethyl-1H-pyrazol-5-yl)thiophene-2-sulphonic acid [4-chloro-3-(3-phenylureido)phenyl]amide

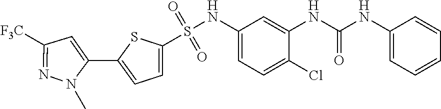

From 5-(1-methyl-3-trifluoromethyl-1H-pyrazol-5-yl)thiophene-2-sulphonyl chloride and 1-(5-amino-2-chlorophenyl)-3-phenyl urea (Intermediate 81)

LCMS (Method C) Rt 12.12 (M+H⁺) 556

¹H NMR (400 MHz) (DMSO-d₆) δ 10.7 (br s, 1H) 9.4 (s, 1H) 8.3 (s, 1H) 8.1 (s, 1H) 7.7 (d, 1H) 7.6 (d, 1H) 7.5 (d, 2H) 7.4 (d, 1H) 7.3 (t, 2H) 7.2 (s, 1H) 7.0 (t, 1H) 6.8 (dd, 1H) 4.0 (s, 3H)

### Example 27

### 1-(4-Fluorophenyl)-1H-pyrazole-4-sulphonic acid N-methyl-N-[4-(3-phenylureido)phenyl]-amide

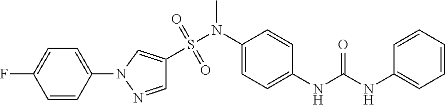

From 1-(4-fluorophenyl)-1H-pyrazole-4-sulphonyl chloride (Intermediate 87) and 1-(4-methylaminophenyl)-3-phenyl urea (Intermediate 25)

LCMS (Method C) Rt 11.34 (M+H⁺) 466

¹H NMR (400 MHz) (DMSO-d₆) δ 9.0 (s, 1H) 8.8 (s, 1H) 8.7 (s, 1H) 8.0 (m, 2H) 7.8 (s, 1H) 7.4 (m, 6H) 7.3 (t, 2H) 7.1 (d, 2H) 7.0 (t, 1H) 3.2 (s, 3H)

### Example 28

### 1-(5-Trifluoromethylpyridin-2-yl)-1H-pyrazole-4-sulphonic acid N-methyl-N-[4-(3-phenyl-ureido)phenyl]amide

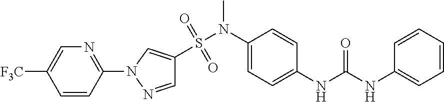

From 1-(5-trifluoromethylpyridin-2-yl)-1H-pyrazole-4-sulphonyl chloride and 1-(4-methyl-aminophenyl)-3-phenyl urea (Intermediate 25)

LCMS (Method C) Rt 12.16 (M+H⁺) 517

¹H NMR (400 MHz) (DMSO-d₆) δ 9.0 (s, 1H) 8.9 (s, 1H) 8.8 (s, 1H) 8.7 (s, 1H) 8.5 (dd, 1H) 8.2 (dd, 1H) 8.0 (s, 1H) 7.4 (dd, 4H) 7.3 (t, 2H) 7.1 (d, 2H) 7.0 (t, 1H) 3.2 (s, 3H)

### Example 29

### 3,5-Dichlorophenylsulphonic acid N-methyl-N-[4-(3-phenylureido)phenyl]amide

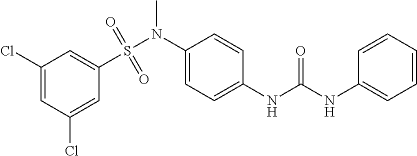

From and 3,5-dichlorophenylsulphonyl chloride and 1-(4-methylaminophenyl)-3-phenyl urea (Intermediate 25)

LCMS (Method C) Rt 12.43 (M+H⁺) 450

¹H NMR (400 MHz) (DMSO-d₆) δ 8.8 (s, 1H) 8.7 (s, 1H) 8.1 (s, 1H) 7.4 (m, 6H) 7.3 (t, 2H) 7.0 (d, 2H) 6.9 (t, 1H) 3.2 (s, 3H)

### Example 30

### 5-(Oxazol-5-yl)thiophene-2-sulphonic acid N-methyl-N-[4-(3-phenylureido)phenyl]amide

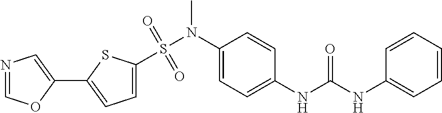

From 5-(oxazol-5-yl)thiophene-2-sulphonyl chloride and 1-(4-methylaminophenyl)-3-phenyl urea (Intermediate 25)

LCMS (Method C) Rt 10.52 (M+H⁺) 455

¹H NMR (400 MHz) (DMSO-d₆) δ 8.8 (s, 1H) 8.7 (s, 1H) 8.5 (s, 1H) 7.8 (s, 1H) 7.6 (d, 1H) 7.5 (d, 1H) 7.4 (m, 4H) 7.3 (t, 2H) 7.1 (d, 2H) 7.0 (t, 1H) 3.2 (s, 3H)

### Example 31

### 5-Methyl-1-phenyl-1H-pyrazole-4-sulphonic acid N-methyl-N-[4-(3-phenylureido)phenyl]-amide

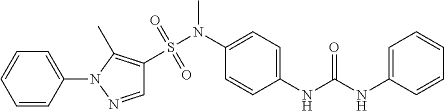

From 5-methyl-1-phenyl-1H-pyrazole-4-sulphonyl chloride and 1-(4-methylaminophenyl)-3-phenyl urea (Intermediate 25) using pyridine in DCM

LCMS (Method C) Rt 11.10 (M+H⁺) 462

¹H NMR (400 MHz) ((DMSO-d₆) δ 8.8 (s, 1H) 8.7 (s, 1H) 7.8 (s, 1H) 7.55 (m, 5H) 7.45 (m, 4H) 7.3 (t, 2H) 7.1 (d, 2H) 6.95 (t, 1H) 3.1 (s, 3H) 1.9 (s, 3H)

### Example 32

### 5-(Pyridin-2-yl)thiophene-2-sulphonic acid [4-(3-phenylureido)phenyl]amide

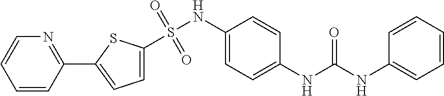

From 5-(pyridin-2-yl)thiophene-2-sulphonyl chloride and 1-(4-aminophenyl)-3-phenyl urea (Intermediate 23)

LCMS (Method C) Rt 10.31 (M+H⁺) 450

¹H NMR (400 MHz) (DMSO-d₆) δ 10.2 (br s, 1H) 8.8 (2s, 2H) 8.7 (d, 1H) 8.0 (d, 1H) 7.9 (t, 1H) 7.8 (d, 1H) 7.5 (d, 1H) 7.4 (m, 5H) 7.3 (t, 2H) 7.1 (d, 2H) 6.9 (t, 1H)

### Example 33

### 5-(1-Methyl-3-trifluoromethyl-1H-pyrazol-5-yl)thiophene-2-sulphonic acid [4-(3-phenyl-ureido)phenyl]amide

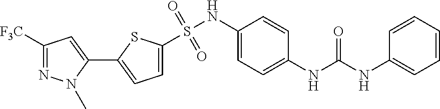

From 5-(1-methyl-3-trifluoromethyl-1H-pyrazol-5-yl)thiophene-2-sulphonyl chloride and 1-(4-aminophenyl)-3-phenyl urea (Intermediate 23)

LCMS (Method C) Rt 11.36 (M+H⁺) 522

¹H NMR (400 MHz) (DMSO-d₆) δ 10.6 (br s, 1H) 8.65 (s, 1H) 8.6 (s, 1H) 7.5 (dd, 2H) 7.4 (dd, 4H) 7.3 (t, 2H) 7.2 (s, 1H) 7.1 (d, 2H) 6.9 (t, 1H) 4.0 (s, 3H)

### Example 34

### 5-(Pyridin-2-yl)thiophene-2-sulphonic acid N-methyl-N-[4-(3-phenylureido)phenyl]amide

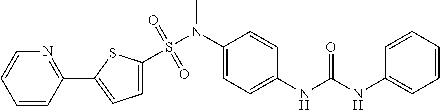

From 5-(pyridin-2-yl)thiophene-2-sulphonyl chloride and 1-(4-methylaminophenyl)-3-phenyl urea (Intermediate 25)

LCMS (Method C) Rt 11.27 (M+H⁺) 465

¹H NMR (400 MHz) (DMSO-d₆) δ 8.8 (s, 1H) 8.7 (s, 1H) 8.6 (d, 1H) 8.1 (d, 1H) 7.9 (m, 2H) 7.5 (m, 6H) 7.3 (t, 2H) 7.1 (d, 2H) 7.0 (t, 1H) 3.2 (s, 3H)

### Example 35

### 3,5-Dimethyl-1-(pyridin-2-yl)-1H-pyrazole-4-sulphonic acid N-methyl-N-[4-(3-phenyl-ureido)phenyl]-amide

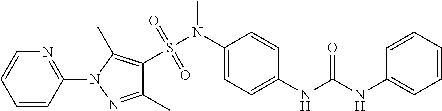

From 3,5-dimethyl-1-(pyridin-2-yl)-1H-pyrazole-4-sulphonyl chloride (Intermediate 88) and 1-(4-methylaminophenyl)-3-phenyl urea (Intermediate 25)

LCMS (Method C) Rt 10.90 (M+H⁺) 477

¹H NMR (400 MHz) (DMSO-d₆) δ 8.8 (s, 1H) 8.7 (s, 1H) 8.6 (dd, 1H) 8.1 (t, 1H) 7.8 (d, 1H) 7.5 (m, 5H) 7.3 (t, 2H) 7.2 (d, 2H) 7.0 (t, 1H) 3.2 (s, 3H) 2.4 (s, 3H) 2.0 (s, 3H)

### Example 36

### 1-(Pyridin-2-yl)-1H-pyrazole-4-sulphonic acid N-methyl-N-[4-(3-phenylureido)phenyl]-amide

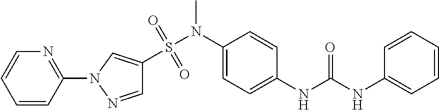

From 1-(pyridin-2-yl)-1H-pyrazole-2-sulphonyl chloride (Intermediate 89) and 1-(4-methylaminophenyl)-3-phenyl urea (Intermediate 25)

LCMS (Method C) Rt 10.89 (M+H⁺) 449

¹H NMR (400 MHz) (DMSO-d₆) δ 8.8 (s, 2H) 8.7 (s, 1H) 8.55 (d, 1H) 8.1 (m, 1H) 8.0 (d, 1H) 7.9 (s, 1H) 7.5 (m, 1H) 7.4 (m, 4H) 7.3 (t, 2H) 7.1 (d, 2H) 7.0 (t, 1H) 3.2 (s, 3H)

### Example 37

### 5-(Isoxazol-5-yl)thiophene-2-sulphonic acid N-methyl-N-[4-(3-phenylureido)phenyl]amide

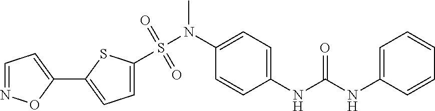

From 5-(isoxazol-5-yl)thiophene-2-sulphonyl chloride and 1-(4-methylaminophenyl)-3-phenyl urea (Intermediate 25)

LCMS (Method C) Rt 11.06 (M+H⁺) 455

¹H NMR (400 MHz) (DMSO-d₆) δ 8.8 (s, 1H) 8.75 (s, 1H) 8.7 (s, 1H) 7.8 (d, 1H) 7.6 (d, 1H) 7.5 (m, 4H) 7.3 (t, 2H) 7.1 (m, 3H) 7.0 (t, 1H) 3.2 (s, 3H)

### Example 38

### 3,5-Dichlorophenylsulphonic acid [4-(3-phenylureido)phenyl]amide

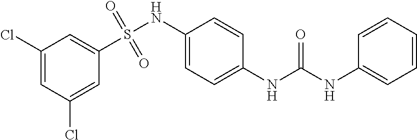

From 3,5-dichlorophenyl sulphonyl chloride and 1-(4-aminophenyl)-3-phenyl urea (Intermediate 23)

LCMS (Method C) Rt 11.45 (M+H⁺) 436

¹H NMR (400 MHz) (DMSO-d₆) δ 10.2 (br s, 1H) 8.65 (s, 1H) 8.6 (s, 1H) 8.0 (s, 1H) 7.65 (s, 2H) 7.45 (d, 2H) 7.4 (d, 2H) 7.3 (t, 2H) 7.0 (m, 3H)

### Example 39

### 4′-Fluorobiphenyl-3-sulphonic acid [3-methoxy-4-(3-phenylureido)phenyl]amide

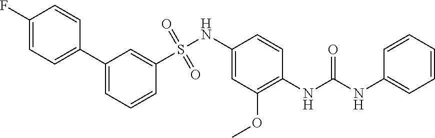

From 4′-fluorobiphenyl-3-sulphonic acid and 1-(4-amino-2-methoxyphenyl)-3-phenyl urea (Intermediate 72)

LCMS (Method C) Rt 11.88 (M+H⁺) 492

¹H NMR (400 MHz) (DMSO-d₆) δ 10.0 (s, 1H) 9.2 (s, 1H) 8.1 (s, 1H) 7.9 (m, 3H) 7.7 (m, 4H) 7.4 (d, 2H) 7.3 (t, 2H) 7.2 (t, 2H) 7.0 (t, 1H) 6.8 (s, 1H) 6.6 (d, 1H) 3.7 (s, 3H)

### Example 40

### 2-Chloro-4-methylphenylsulphonic acid N-methyl-N-[4-(3-phenylureido)phenyl]amide

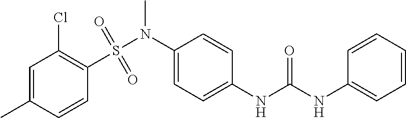

From 2-chloro-4-methylphenylsulphonyl chloride and 1-(4-methylaminophenyl)-3-phenyl urea (Intermediate 25)

LCMS (Method C) Rt 11.66 (M+H⁺) 430

¹H NMR (400 MHz) (DMSO-d₆) δ 8.8 (s, 1H) 8.7 (s, 1H) 7.7 (d, 1H) 7.5 (s, 1H) 7.4 (m, 4H) 7.3 (m, 3H) 7.1 (d, 2H) 7.0 (t, 1H) 3.3 (s, 3H) 2.4 (s, 3H)

### Example 41

### 2,3-Dichlorophenylsulphonic acid N-methyl-N-[4-(3-phenylureido)phenyl]amide

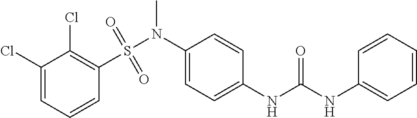

From 2,3-dichlorophenylsulphonyl chloride and 1-(4-methylaminophenyl)-3-phenyl urea (Intermediate 25)

LCMS (Method C) Rt 11.86 (M+H⁺) 450

¹H NMR (400 MHz) (DMSO-d₆) δ 8.8 (s, 1H) 8.7 (s, 1H) 8.0 (d, 1H) 7.8 (d, 1H) 7.5 (t, 1H) 7.4 (m, 4H) 7.3 (t, 2H) 7.1 (d, 2H) 7.0 (t, 1H) 3.3 (s, 3H)

### Example 42

### 5-(1-Methyl-3-trifluoromethyl-1H-pyrazol-5-yl)thiophene-2-sulphonic acid N-methyl-N-[4-(3-phenylureido)phenyl]amide

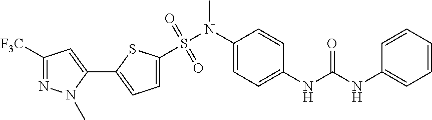

From 5-(1-methyl-3-trifluoromethyl-1H-pyrazole-5-yl)thiophene-2-sulphonyl chloride and 1-(4-methylaminophenyl)-3-phenyl urea (Intermediate 25)

LCMS (Method C) Rt 12.20 (M+H⁺) 536

¹H NMR (400 MHz) (DMSO-d₆) δ 8.9 (s, 1H) 8.7 (s, 1H) 7.7 (d, 1H) 7.6 (d, 1H) 7.4 (d, 4H) 7.3 (t, 2H) 7.2 (s, 1H) 7.1 (d, 2H) 6.9 (t, 1H) 4.0 (s, 3H) 3.2 (s, 3H)

### Example 43

### 5-(5-Trifluoromethylisoxazol-3-yl)thiophene-2-sulphonic acid N-methyl-N-[4-(3-phenyl-ureido)phenyl]amide

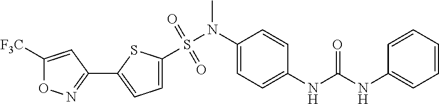

From 5-(5-trifluoromethylisoxazol-3-yl)thiophene-2-sulphonyl chloride and 1-(4-methylaminophenyl)-3-phenyl urea (Intermediate 25)

LCMS (Method C) Rt 12.70 (M+H⁺) 523

¹H NMR (400 MHz) (DMSO-d₆) δ 8.8 (s, 1H) 8.6 (s, 1H) 8.2 (s, 1H) 8.0 (d, 1H) 7.7 (d, 1H) 7.5 (m, 4H) 7.3 (t, 2H) 7.1 (d, 2H) 7.0 (t, 1H) 3.2 (s, 3H)

### Example 44

### 5-(1-Methyl-5-trifluoromethyl-1H-pyrazol-3-yl)thiophene-2-sulphonic acid N-methyl-N-[4-(3-phenylureido)phenyl]amide

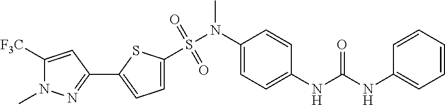

From 5-(1-methyl-5-trifluoromethyl-1H-pyrazol-3-yl)thiophene-2-sulphonyl chloride and 1-(4-methylaminophenyl)-3-phenyl urea (Intermediate 25)

LCMS (Method C) Rt 12.49 (M+H⁺) 536

¹H NMR (400 MHz) (DMSO-d₆) δ 8.8 (s, 1H) 8.7 (s, 1H) 7.7 (d, 1H) 7.6 (s, 1H) 7.5 (m, 5H) 7.3 (t, 2H) 7.1 (d, 2H) 7.0 (t, 1H) 4.0 (s, 3H) 3.2 (s, 3H)

### Example 45

### 5-(2-Methylthiazol-4-yl)thiophene-2-sulphonic acid N-methyl-N-[4-(3-phenylureido)-phenyl]amide

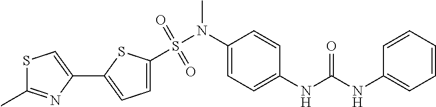

From 5-(2-methylthiazol-4-yl)thiophene-2-sulphonyl chloride and 1-(4-methylaminophenyl)-3-phenyl urea (Intermediate 25)

LCMS (Method C) Rt 11.61 (M+H⁺) 485

¹H NMR (400 MHz) (DMSO-d₆) δ 8.8 (s, 1H) 8.7 (s, 1H) 8.1 (s, 1H) 7.65 (d, 1H) 7.45 (m, 5H) 7.3 (t, 2H) 7.1 (d, 2H) 6.95 (t, 1H) 3.2 (s, 3H) 2.7 (s, 3H)

### Example 46

### 5-(5-Methyl-1,3,4-oxadiazol-2-yl)thiophene-2-sulphonic acid N-methyl-N-[4-(3-phenyl-ureido)phenyl]amide

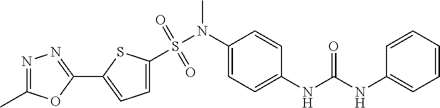

From 5-(5-methyl-1,3,4-oxadiazol-2-yl)thiophene-2-sulphonyl chloride and 1-(4-methylaminophenyl)-3-phenyl urea (Intermediate 25)

LCMS (Method C) Rt 10.23 (M+H⁺) 470

¹H NMR (400 MHz) (DMSO-d₆) δ 8.8 (s, 1H) 8.7 (s, 1H) 7.9 (d, 1H) 7.6 (d, 1H) 7.5 (m, 4H) 7.3 (t, 2H) 7.1 (d, 2H) 7.0 (t, 1H) 3.2 (s, 3H) 2.6 (s, 3H)

### Example 47

### 1-(5-Fluoropyridin-2-yl)-1H-pyrazole-4-sulphonic acid N-methyl-N-[4-(3-phenylureido)-phenyl]amide

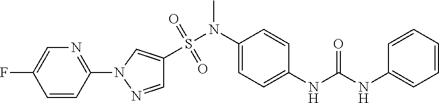

From 1-(5-fluoropyridin-2-yl)-1H-pyrazole-4-sulphonyl chloride (Intermediate 90) and 1-(4-methylaminophenyl)-3-phenyl urea (Intermediate 25)

LCMS (Method C) Rt 11.20 (M+H⁺) 467

¹H NMR (400 MHz) (DMSO-d₆) δ 8.8 (s, 1H) 8.75 (s, 1H) 8.7 (s, 1H) 8.6 (s, 1H) 8.1 (d, 2H) 7.9 (s, 1H) 7.5 (m, 4H) 7.3 (t, 2H) 7.1 (d, 2H) 6.9 (t, 1H) 3.1 (s, 3H)

### Example 48

### 4′-Fluorobiphenyl-3-sulphonic acid [4-(3-phenylureido)phenyl]amide

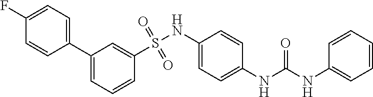

From 4′-fluorobiphenyl-3-sulphonyl chloride and 1-(4-aminophenyl)-3-phenyl urea (Intermediate 23) using N,N-diisopropyl-N-ethylamine in DCM

LCMS (Method C) Rt 11.26 (M+H⁺) 462

¹H NMR (400 MHz) (DMSO-d₆) δ 10.0 (br s, 1H) 8.6 (s, 1H) 8.55 (s, 1H) 7.9 (m, 2H) 7.7 (m, 4H) 7.4 (d, 2H) 7.3 (m, 6H) 7.0 (d, 2H) 7.95 (t, 1H)

### Example 49

### 3-tert-Butylphenylsulphonic acid N-methyl-N-[4-(3-phenylureido)phenyl]amide

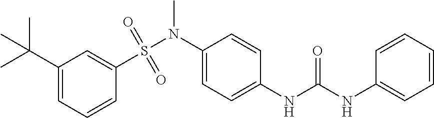

From 3-tert-butylphenylsulphonyl chloride and 1-(4-methylaminophenyl)-3-phenyl urea (Intermediate 25) using pyridine in DCM

LCMS (Method C) Rt 12.50 (M+H⁺) 438

¹H NMR (400 MHz) (DMSO-d₆) δ 8.9 (s, 1H) 8.8 (s, 1H) 7.8 (d, 1H) 7.6 (t, 1H) 7.4 (m, 5H) 7.3 (m, 3H) 6.9 (m, 3H) 3.05 (s, 3H) 1.2 (s, 9H)

### Example 50

### 3-Cyanophenylsulphonic acid N-methyl-N-[4-(3-phenylureido)phenyl]amide

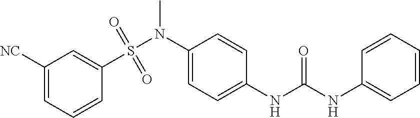

From 3-cyanophenylsuphonyl chloride and 1-(4-methylaminophenyl)-3-phenyl urea (Intermediate 25) using pyridine in DCM

LCMS (Method C) Rt 10.70 (M+H⁺) 407

¹H NMR (400 MHz) (DMSO-d₆) δ 8.9 (s, 1H) 8.8 (s, 1H) 8.2 (d, 1H) 8.0 (s, 1H) 7.8 (m, 2H) 7.4 (m, 4H) 7.3 (t, 2H) 7.0 (m, 3H) 3.1 (s, 3H)

### Example 51

### 4′-Fluorobiphenyl-3-sulphonic acid [4-chloro-3-(3-phenylureido)phenyl]amide

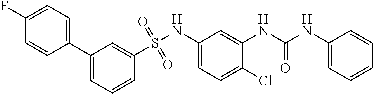

From 4′-fluorobiphenyl-3-sulphonyl chloride and 1-(5-amino-2-chlorophenyl)-3-phenyl urea (Intermediate 81) using N,N-diisopropyl-N-ethylamine in NMP

LCMS (Method C) Rt 12.40 (M+H⁺) 496

¹H NMR (400 MHz) (DMSO-d₆) δ 10.5 (br s, 1H) 9.4 (s, 1H) 8.35 (s, 1H) 8.3 (s, 1H) 8.1 (s, 1H) 7.9 (d, 1H) 7.7 (m, 3H) 7.65 (t, 1H) 7.4 (d, 2H) 7.3 (m, 3H) 7.2 (t, 2H) 7.0 (t, 1H) 6.8 (d, 1H)

### Example 52

### 4′-Fluorobiphenyl-3-sulphonic acid [2-(3-phenylureido)pyridin-5-yl]-amide

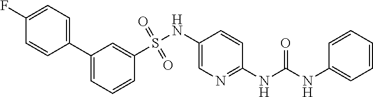

From 4′-fluorobiphenyl-3-sulphonyl chloride and 1-(5-aminopyridin-2-yl)-3-phenyl urea (Intermediate 66) using pyridine in DCM

LCMS (Method C) Rt 11.43 (M+H⁺) 463

¹H NMR (400 MHz) (DMSO-d₆) δ 10.2 (br s, 1H) 9.9 (s, 1H) 9.3 (s, 1H) 7.9 (m, 3H) 7.7 (m, 4H) 7.5 (m, 4H) 7.3 (m, 4H) 7.0 (t, 1H)

### Example 53

### 4′-Fluorobiphenyl-3-sulphonic acid [2-methyl-5-(3-phenylureido)phenyl]amide

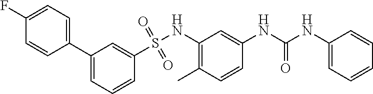

From 4′-fluorobiphenyl-3-sulphonyl chloride and 1-(3-amino-4-methylphenyl)-3-phenyl urea (Intermediate 63) using N,N-diisopropyl-N-ethylamine in NMP

LCMS (Method C) Rt 11.90 (M+H⁺) 476

¹H NMR (400 MHz) (DMSO-d₆) δ 9.6 (s, 1H) 8.6 (s, 1H) 8.5 (s, 1H) 7.9 (m, 2H) 7.7 (m, 4H) 7.4 (d, 2H) 7.3 (m, 5H) 7.15 (d, 1H) 7.0 (d, 1H) 6.95 (t, 1H) 1.95 (s, 3H)

### Example 54

### 4′-Fluorobiphenyl-3-sulphonic acid [4-(3-benzylureido)phenyl]amide

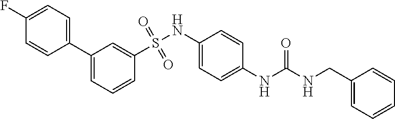

From 4′-fluorobiphenyl-3-sulphonyl chloride and 1-(4-aminophenyl)-3-benzyl urea (Intermediate 26) using pyridine in DCM

LCMS (Method C) Rt 11.28 (M+H⁺) 476

¹H NMR (400 MHz) (DMSO-d₆) δ 9.9 (br s, 1H) 8.6 (s, 1H) 7.9 (m, 2H) 7.6 (m, 4H) 7.4-7.2 (m, 9H) 6.95 (d, 2H) 6.6 (t, 1H) 4.3 (d, 2H)

### Example 55

### 5-Bromothiophene-2-sulphonic acid N-methyl-N-[4-(3-phenylureido)phenyl]amide

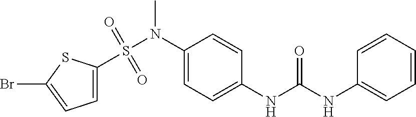

From 5-bromothiophene-2-sulphonyl chloride and 1-(4-methylaminophenyl)-3-phenyl urea (Intermediate 25) using pyridine in DCM.

LCMS (Method C) Rt 10.63 (M+H)⁺388

¹H NMR (400 MHz) (DMSO-d₆) δ 8.8 (s, 1H) 8.7 (s, 1H) 8.0 (d, 1H) 7.5 (m, 4H) 7.3 (m, 3H) 7.0 (m, 3H) 3.1 (s, 3H)

### Example 56

### 2-(4-Fluorophenyl)-1-methyl-1H-imidazole-4-sulphonic acid N-methyl-N-[4-(3-phenyl-ureido)phenyl]amide

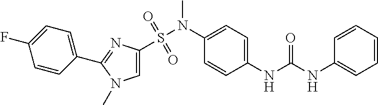

From 2-(4-fluorophenyl)-1-methyl-1H-imidazole-4-sulphonyl chloride (Intermediate 91) and 1-(4-methylaminophenyl)-3-phenyl urea (Intermediate 25) using pyridine in DCM

LCMS (Method C) Rt 10.54 (M+H⁺) 480

¹H NMR (400 MHz) ((DMSO-d₆) δ 8.8 (s, 1H) 8.7 (s, 1H) 7.9 (m, 3H) 7.4 (m, 6H) 7.3 (t, 2H) 7.15 (d, 2H) 6.95 (t, 1H) 3.8 (s, 3H) 3.25 (s, 3H)

### Example 57

### 6-[5-(1-Methyl-3-trifluoromethyl-1H-pyrazol-5-yl)thiophene-2-sulphonylamino]-2,3-dihydroindole-1-carboxylic acid N-phenylamide

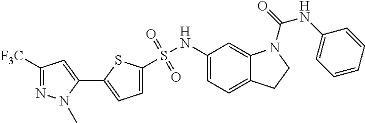

From 5-(1-methyl-3-trifluoromethyl-1H-pyrazol-5-yl)thiophene-2-sulphonyl chloride and 6-amino-2,3-dihydroindole-1-carboxylic acid N-phenylamide (Intermediate 69) using pyridine in DCM

LCMS (Method C) Rt 11.82 (M+H⁺) 450

¹H NMR (400 MHz) (DMSO-d₆) δ 10.4 (br s, 1H) 8.5 (s, 1H) 7.8 (s, 1H) 7.6 (d, 1H) 7.5 (m, 3H) 7.3 (t, 2H) 7.15 (s, 1H) 7.1 (d, 1H) 7.0 (t, 1H) 6.7 (d, 1H) 4.1 (t, 2H) 4.0 (s, 3H) 3.1 (t, 2H)

### Example 58

### 5-(1-Methyl-3-trifluoromethyl-1H-pyrazol-5-yl)thiophene-2-sulphonic acid [4-methoxy-3-(3-phenylureido)phenyl]amide

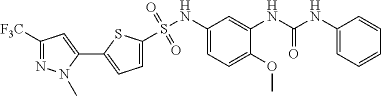

From 5-(1-methyl-3-trifluoromethyl-1H-pyrazol-5-yl)thiophene-2-sulphonyl chloride and 1-(5-amino-2-methoxyphenyl)-3-phenyl urea (Intermediate 73) using pyridine in DCM

LCMS (Method C) Rt 11.74 (M+H⁺) 552

¹H NMR (400 MHz) (DMSO-d₆) δ 10.2 (br s, 1H) 9.3 (s, 1H) 8.2 (s, 1H) 8.0 (s, 1H) 7.5 (dd, 2H) 7.4 (d, 2H) 7.3 (t, 2H) 7.1 (s, 1H) 6.9 (m, 2H) 6.8 (dd, 1H) 4.0 (s, 3H) 3.8 (s, 3H)

### Example 59

### 2-Chloro-4-methylthiazole-5-sulphonic acid N-methyl-N-[4-(3-phenylureido)phenyl]amide

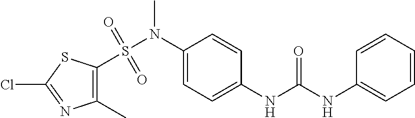

From 2-chloro-4-methylthiazole-5-sulphonyl chloride and 1-(4-methylaminophenyl)-3-phenyl urea (Intermediate 25) using pyridine in DCM

LCMS (Method C) Rt 11.51 (M+H⁺) 437

¹H NMR (400 MHz) (DMSO-d₆) δ 8.9 (s, 1H) 8.7 (s, 1H) 7.5 (m, 4H) 7.3 (t, 2H) 7.2 (d, 2H) 7.0 (t, 1H) 3.2 (s, 3H) 2.1 (s, 3H)

### Example 60

### 4′-Fluorobiphenyl-3-sulphonic acid [2-chloro-4-(3-phenylureido)phenyl]amide

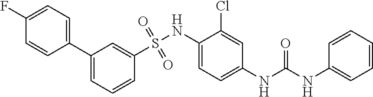

From 4′-fluorobiphenyl-3-sulphonyl chloride and 1-(4-amino-3-chlorophenyl)-3-phenyl urea (Intermediate 85) using pyridine in DCM

LCMS (Method C) Rt 12.12 (M+H⁺) 496

¹H NMR (400 MHz) (DMSO-d₆) δ 10.0 (br s, 1H) 8.9 (s, 1H) 8.8 (s, 1H) 7.9 (m, 2H) 7.7 (m, 5H) 7.5 (d, 2H) 7.3 (m, 4H) 7.2 (m, 2H) 7.0 (t, 1H)

### Example 61

### 5-(1-Methyl-3-trifluoromethyl-1H-pyrazol-5-yl)thiophene-2-sulphonic acid [3-chloro-4-(3-phenylureido)phenyl]amide

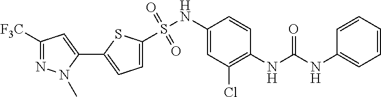

From 5-(1-methyl-3-trifluoromethyl-1H-pyrazol-5-yl)thiophene-2-sulphonyl chloride and 1-(4-amino-2-chlorophenyl)-3-phenyl urea (Intermediate 83) using pyridine in DCM

LCMS (Method C) Rt 12.10 (M+H⁺) 556

¹H NMR (400 MHz) (DMSO-d₆) δ 10.7 (br s, 1H) 9.3 (s, 1H) 8.3 (s, 1H) 8.1 (d, 1H) 7.7 (d, 1H) 7.6 (d, 1H) 7.5 (d, 2H) 7.3 (t, 2H) 7.2 (d, 2H) 7.1 (d, 1H) 7.0 (t, 1H) 4.0 (s, 3H)

### Example 62

### 5-Chlorothiophene-2-sulphonic acid N-methyl-N-[4-(3-phenylureido)phenyl]amide

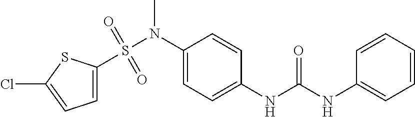

From 5-chlorothiophene-2-sulphonyl chloride and 1-(4-methylaminophenyl)-3-phenyl urea (Intermediate 25) using pyridine in DCM

LCMS (Method C) Rt 11.69 (M+H⁺) 421

¹H NMR (400 MHz) (DMSO-d₆) δ 8.9 (s, 1H) 8.7 (s, 1H) 7.5 (m, 4H) 7.4 (d, 1H) 7.35 (d, 1H) 7.3 (t, 2H) 7.1 (d, 2H) 7.0 (t, 1H) 3.1 (s, 3H)

### Example 63

### 2,3-Dihydrobenzo-1,4-dioxin-6-sulphonic acid N-methyl-N-[4-(3-phenylureido)phenyl]-amide

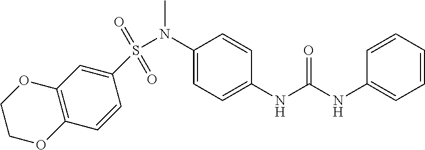

From 2,3-dihydrobenzo-1,4-dioxin-6-sulphonyl chloride and 1-(4-methylaminophenyl)-3-phenyl urea (Intermediate 25) using pyridine in DCM

LCMS (Method C) Rt 10.70 (M+H⁺) 440

¹H NMR (400 MHz) (DMSO-d₆) δ 8.8 (s, 1H) 8.7 (s, 1H) 7.4 (m, 4H) 7.3 (t, 2H) 7.0 (m, 6H) 4.35 (t, 2H) 4.3 (t, 2H) 3.1 (s, 3H)

### Example 64

### 3-Methoxyphenyl sulphonic acid N-methyl-N-[4-(3-phenylureido)phenyl]amide

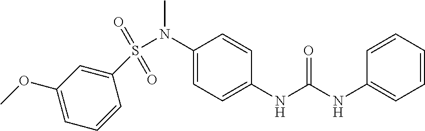

From 3-methoxyphenylsulphonyl chloride and 1-(4-methylaminophenyl)-3-phenyl urea (Intermediate 25) using pyridine in DCM

LCMS (Method C) Rt 10.93 (M+H⁺) 412

¹H NMR (400 MHz) (DMSO-d₆) 8.8 (s, 1H) 8.7 (s, 1H) 7.5 (t, 1H) 7.4 (m, 4H) 7.3 (t, 3H) 7.1 (d, 1H) 7.0 (m, 3H) 6.9 (s, 1H) 3.8 (s, 3H) 3.1 (s, 3H)

### Example 65

### 4′-Fluorobiphenyl-3-sulphonic acid [3-(3-phenylureido)phenyl]amide

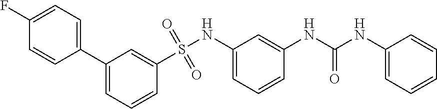

From 4′-fluorobiphenyl-3-sulphonyl chloride and 1-(3-aminophenyl)-3-phenyl urea (Intermediate 24) using pyridine in DCM

LCMS (Method C) Rt 11.68 (M+H⁺) 462

¹H NMR (400 MHz) (DMSO-d₆) δ 10.3 (br s, 1H) 8.7 (s, 1H) 8.6 (s, 1H) 8.0 (s, 1H) 7.9 (d, 1H) 7.7 (m, 3H) 7.6 (t, 1H) 7.5 (s, 1H) 7.4 (d, 2H) 7.3 (m, 4H) 7.1 (t, 1H) 7.0 (m, 2H) 6.7 (d, 1H)

### Example 66

### 4′-Fluorobiphenyl-3-sulphonic acid N-(2-hydroxyethyl)-N-[4-(3-phenylureido)phenyl]amide

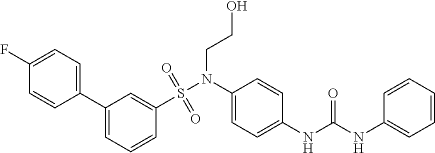

To an ice-cold solution of {N-(4′-fluorobiphenyl-3-sulphonyl)-N-[4-(3-phenylureido)-phenyl]amino}acetic acid ethyl ester (Intermediate 39, 125 mg) in THF (5 ml) was slowly added a solution of LiAlH₄ in THF (1M, 250 μl). The reaction mixture was stirred for 40 minutes at 0° C. then it was quenched by addition of ethyl acetate (2 ml) followed by water (2 ml), NaOH (1M, 2 ml) and finally saturated aqueous NH₄Cl (2 ml). The resultant mixture was stirred for 10 minutes then more ethyl acetate was added. The organic layer was separated, dried (Na₂SO₄) and filtered. The volatiles were removed by evaporation and the residue was purified by HPLC eluting with a mixture of water and acetonitrile (each containing 0.1% formic acid) from 20 to 98% acetonitrile over 25 minutes to give 4′-fluorobiphenyl-3-sulphonic acid N-(2-hydroxyethyl)-N-[4-(3-phenylureido)phenyl]amide (70 mg).

LCMS (Method C) Rt 11.18 (M+H⁺) 506

¹H NMR (400 MHz) (DMSO-d₆) δ 8.8 (br s, 1H) 8.7 (br s, 1H) 8.0 (d, 1H) 7.7 (m, 4H) 7.6 (d, 1H) 7.4 (m, 4H) 7.3 (m, 4H) 7.0 (m, 3H) 4.8 (t, 1H) 3.6 (t, 2H) 3.4 (m, 2H).

### Example 67

### 5-(4-Fluorophenyl)pyridine-3-sulphonic acid N-methyl-N-[4-(3-phenylureido)phenyl]amide

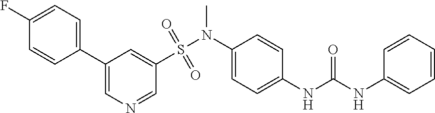

A mixture of 5-bromopyridine-3-sulphonic acid N-methyl-N-[4-(3-phenylureido)-phenyl]amide (Intermediate 6, 60 mg), 4-fluorophenyl boronic acid (36 mg), [1,1-Bis-(diphenylphosphino)ferrocene]dichloropalladium (II) (10 mg) and cesium carbonate (84 mg) in DME (1 ml) and IMS (1 ml) was heated in the microwave at 150° C. for 2 minutes. The reaction mixture was partitioned between water and ethyl acetate. The organic layer was dried (MgSO₄), filtered and the volatiles were removed by evaporation. The residue was purified by HPLC eluting with a mixture of water and acetonitrile (each containing 0.1% formic acid) from 20 to 95% acetonitrile over 25 minutes to give 5-(4-fluorophenyl)pyridine-3-sulphonic acid N-methyl-N-[4-(3-phenylureido)phenyl]amide (20 mg).

LCMS (Method C) Rt 11.50 (M+H⁺) 477

¹H NMR (400 MHz) (DMSO-d₆) δ 9.2 (s, 1H) 8.8 (s, 1H) 8.7 (s, 1H) 8.6 (s, 1H) 8.0 (s, 1H) 7.8 (m, 2H) 7.4 (m, 4H) 7.3 (t, 2H) 7.2 (t, 2H) 7.1 (d, 2H) 7.0 (t, 1H) 3.2 (s, 3H)

By proceeding in a similar manner the following examples were prepared from the appropriate starting materials. The reaction may also be performed using different catalysts, bases and solvents.

### Example 68

### 3-(Pyridin-3-yl)phenylsulphonic acid N-methyl-N-[4-(3-phenylureido)phenyl]amide

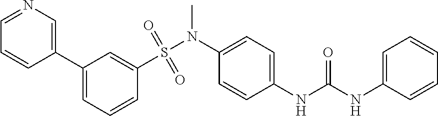

From 3-pyridylboronic acid and 3-bromophenylsulphonic acid N-methylN-[4-(3-phenylureido)phenyl]amide (Intermediate 7) using [1,1′-bis(diphenylphosphino)ferrocene]-dichloropalladium (II) and aqueous Na₂CO₃ (2M) in acetonitrile.

LCMS (Method C) Rt 8.99 (M+H⁺) 459

¹H NMR (400 MHz) ((DMSO-d₆) δ 8.9 (m, 2H) 8.8 (s, 1H) 8.7 (d, 1H) 8.1 (t, 2H) 7.8 (m, 2H) 7.6 (m, 2H) 7.4 (m, 4H) 7.2 (t, 2H) 7.0 (d, 2H) 6.95 (t, 1H) 3.2 (s, 3H)

### Example 69

### 5-(1H-Pyrazol-4-yl)thiophene-2-sulphonic acid N-methyl-N-[4-(3-phenylureido)phenyl]-amide

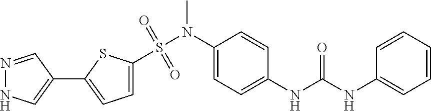

From 1H-pyrazole-4-boronic acid pinacol ester and 5-bromothiophene-2-sulphonic acid N-methyl-N-[4-(3-phenylureido)phenyl]amide (Example 55) using tetrakis(triphenyl-phosphine)palladium(0) and aqueous Na₂CO₃ (2M) in DME.

LCMS (Method C) Rt 9.78 (M+H⁺) 454

¹H NMR (400 MHz) (DMSO-d₆) δ 13.2 (br s, 1H) 8.9 (s, 1H) 8.8 (s, 1H) 8.3 (s, 1H) 7.9 (s, 1H) 7.5 (m, 4H) 7.3 (m, 4H) 7.1 (d, 2H) 6.9 (t, 1H) 3.3 (s, 3H)

### Example 70

### 5-(1-Methyl-1H-pyrazol-4-yl)thiophene-2-sulphonic acid N-methyl-N-[4-(3-phenylureido)-phenyl]amide

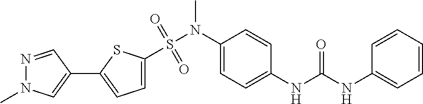

From 1-methyl-1H-pyrazole-4-boronic acid and 5-bromothiophene-2-sulphonic acid N-methyl-N-[4-(3-phenylureido)phenyl]amide (Example 55) using tetrakis(triphenyl-phosphine)palladium(0) and aqueous Na₂CO₃ (2M) in acetonitrile.

LCMS (Method C) Rt 10.40 (M+H⁺) 468

¹H NMR (400 MHz) (DMSO-d₆) δ 8.8 (s, 1H) 8.7 (s, 1H) 8.2 (s, 1H) 7.8 (s, 1H) 7.5 (m, 4H) 7.4 (d, 1H) 7.3 (m, 3H) 7.1 (d, 2H) 7.0 (t, 1H) 3.8 (s, 3H) 3.2 (s, 3H)

### Example 71

### 5-(Pyridin-4-yl)thiophene-2-sulphonic acid N-methyl-N-[4-(3-phenylureido)phenyl]amide

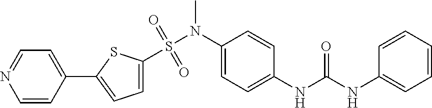

From pyridine-4-boronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME.

LCMS (Method C) Rt 8.63 (M+H⁺) 465

¹H NMR (400 MHz) (DMSO-d₆) δ 8.8 (s, 1H) 8.7 (s, 1H), 8.6 (d, 2H) 7.9 (d, 1H) 7.7 (d, 2H) 7.5 (d, 1H) 7.4 (m, 4H) 7.3 (t, 2H) 7.1 (d, 2H) 6.9 (t, 1H) 3.2 (s, 3H)

### Example 72

### 5-(Pyridin-3-yl)thiophene-2-sulphonic acid N-methyl-N-[4-(3-phenylureido)phenyl]amide

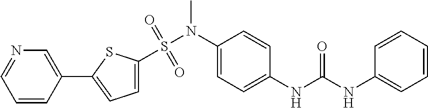

From pyridine-3-boronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME.

LCMS (Method C) Rt 10.07 (M+H⁺) 465

¹H NMR (400 MHz) (DMSO-d₆) δ 9.0 (s, 1H) 8.8 (s, 1H) 8.7 (s, 1H) 8.6 (d, 1H) 8.2 (dd, 1H) 7.8 (d, 1H) 7.5 (m, 6H) 7.3 (t, 2H) 7.1 (d, 2H) 6.9 (t, 1H) 3.2 (s, 3H)

### Example 73

### 5-(Pyrimidin-5-yl)thiophene-2-sulphonic acid N-methyl-N-[4-(3-phenylureido)phenyl]amide

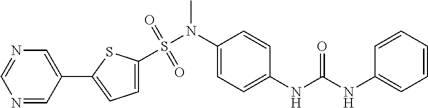

From 5-pyrimidyl boronic acid hydrate and 5-bromothiophene-2-sulphonic acid N-methyl-N-[4-(3-phenylureido)phenyl]amide (Example 55) using tetrakis(triphenylphosphine)-palladium(0) and aqueous Na₂CO₃ (2M) in DME

LCMS (Method C) Rt 10.05 (M+H⁺) 466

¹H NMR (400 MHz) (DMSO-d₆) δ 9.3 (s, 1H) 9.2 (s, 2H) 9.15 (s, 1H) 9.1 (s, 1H) 7.9 (d, 1H) 7.55 (d, 1H) 7.5 (d, 4H) 7.3 (t, 2H) 7.1 (d, 2H) 6.9 (t, 1H) 3.2 (s, 3H)

### Example 74

### 5-(Cyclohexen-1-yl)thiophene-2-sulphonic acid N-methyl-N-[4-(3-phenylureido)phenyl]-amide

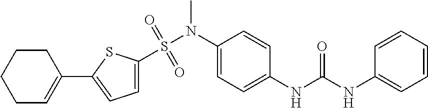

From cyclohexen-1-yl boronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME

LCMS (Method C) Rt 13.41 (M+H⁺) 468

¹H NMR (400 MHz) (DMSO-d₆) δ 9.5 (s, 1H) 9.4 (s, 1H) 7.5 (m, 4H) 7.3 (m, 3H) 7.1 (d, 1H) 7.0 (d, 2H) 7.0 (t, 1H) 6.3 (m, 1H) 3.1 (s, 3H) 2.3 (m, 2H) 2.1 (m, 2H) 1.7 (m, 2H) 1.6 (m, 2H)

### Example 75

### 5-(1-Methyl-1H-pyrazol-5-yl)thiophene-2-sulphonic acid N-methyl-N-[4-(3-phenylureido)-phenyl]amide

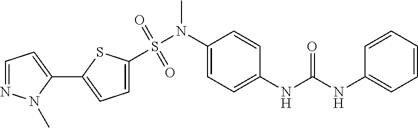

From 1-methyl-1H-pyrazole-5-boronic acid pinacol ester and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME

LCMS (Method C) Rt 10.65 (M+H⁺) 468

¹H NMR (400 MHz) (DMSO-d₆) δ 8.8 (s, 1H) 8.7 (s, 1H) 7.5 (m, 3H) 7.4 (m, 4H) 7.3 (t, 2H) 7.1 (d, 2H) 7.0 (t, 1H) 6.6 (s, 1H) 4.0 (s, 3H) 3.2 (s, 3H)

### Example 76

### 5-(3,5-Dimethylisoxazol-4-yl)thiophene-2-sulphonic acid N-methylN-[4-(3-phenylureido)-phenyl]amide

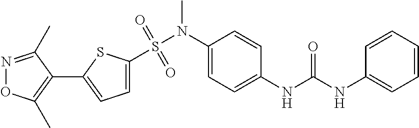

From 3,5-dimethylisoxazol-4-boronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME

LCMS (Method C) Rt 11.43 (M+H⁺) 483

¹H NMR (400 MHz) (DMSO-d₆) δ 8.8 (s, 1H) 8.7 (s, 1H) 7.6 (d, 1H) 7.5 (m, 4H) 7.4 (d, 1H) 7.3 (t, 2H) 7.1 (d, 2H) 7.0 (t, 1H) 3.2 (s, 3H) 2.5 (s, 3H) 2.3 (s, 3H)

### Example 77

### 3-(1-Methyl-3-trifluoromethyl-1H-pyrazole-5-yl)phenylsulphonic acid N-methyl-N-[4-(3-phenylureido)phenyl]amide

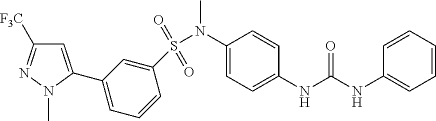

From 1-methyl-3-trifluoromethyl-1H-pyrazol-5-boronic acid and 3-bromophenylsulphonic acid N-methyl-N-[4-(3-phenylureido)phenyl]amide (Intermediate 7) using tetrakis(triphenylphosphine)palladium(0) and aqueous Na₂CO₃ (2M) in DME

LCMS (Method C) Rt 12.12 (M+H⁺) 530

¹H NMR (400 MHz) (DMSO-d₆) δ 8.8 (s, 1H) 8.7 (s, 1H) 8.0 (d, 1H) 7.8 (t, 1H) 7.6 (m, 2H) 7.4 (m, 4H) 7.3 (m, 2H) 7.1 (d, 2H) 7.0 (m, 2H) 3.8 (s, 3H) 3.1 (s, 3H)

### Example 78

### 5-(6-Methoxypyridin-3-yl)thiophene-2-sulphonic acid N-methyl-N-[4-(3-phenylureido)phenyl]amide

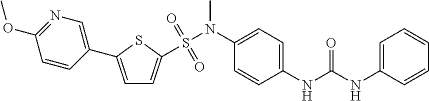

From 2-methoxypyridyl-5-boronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME

LCMS (Method C) Rt 11.94 (M+H⁺) 495

¹H NMR (400 MHz) (DMSO-d₆) δ 8.8 (s, 1H) 8.7 (s, 1H) 8.6 (d, 1H) 8.1 (dd, 1H) 7.6 (d, 1H) 7.5 (m, 5H) 7.3 (t, 2H) 7.1 (d, 2H) 6.9 (m, 2H) 3.9 (s, 3H) 3.1 (s, 3H)

### Example 79

### 3-(1-Methyl-1H-pyrazole-5-yl)phenylsulphonic acid N-methyl-N-[4-(3-phenylureido)phenyl]amide

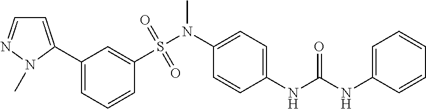

From 1-methyl-1-H-pyrazole-5-boronic acid pinacol ester and 3-bromophenylsulphonic acid N-methyl-N-[4-(3-phenylureido)phenyl]amide (Intermediate 7) using tetrakis(triphenylphosphine)palladium(0) and aqueous Na₂CO₃ (2M) in DME

LCMS (Method C) Rt 10.38 (M+H⁺) 462

¹H NMR (400 MHz) ((DMSO-d₆) δ 8.9 (s, 1H) 8.8 (s, 1H) 7.9 (d, 1H) 7.7 (t, 1H) 7.6 (d, 1H) 7.5 (m, 6H) 7.3 (m, 2H) 7.0 (m, 3H) 6.4 (s, 1H) 3.8 (s, 3H) 3.2 (s, 3H)

### Example 80

### 5-Chloro-4-(1-methyl-1H-pyrazol-5-yl)thiophene-2-sulphonic acid N-methyl-N-[4-(3-phenyl-ureido)phenyl]amide

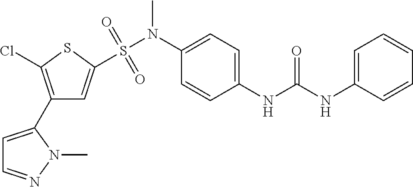

From 1-methyl-1H-pyrazole-5-boronic acid pinacol ester and 5-chloro-4-bromothiophene-2-sulphonic acid N-methyl-N-[4-(3-phenylureido)phenyl]amide (Intermediate 15) using tetrakis(triphenylphosphine)palladium(0) and aqueous Na₂CO₃ (2M) in DME.

LCMS (Method C) Rt 11.32 (M+H⁺) 501

¹H NMR (400 MHz) (DMSO-d₆) δ 8.8 (br s, 1H) 8.7 (br s, 1H) 7.6 (s, 1H) 7.5 (d, 1H) 7.45-7.4 (m, 4H) 7.2 (t, 2H) 7.1 (d, 2H) 6.9 (t, 1H) 6.5 (d, 1H) 3.7 (s, 3H) 3.2 (s, 3H).

### Example 81

### 5-Cyclopropylthiophene-2-sulphonic acid N-methyl-N-[4-(3-phenylureido)phenyl]amide

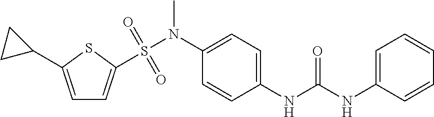

From cyclopropylboronic acid and 5-bromothiophene-2-sulphonic acid N-methyl-N-[4-(3-phenylureido)phenyl]amide (Example 55) using tetrakis(triphenylphosphine)palladium(0) and aqueous Na₂CO₃ (2M) in DME.

LCMS (Method C) Rt 11.77 (M+H⁺) 428

¹H NMR (400 MHz) (DMSO-d₆) δ 8.8 (br s, 1H) 8.7 (br s, 1H) 7.5-7.4 (m, 4H) 7.3 (t, 2H) 7.25 (d, 1H) 7.1 (d, 2H) 7.0 (t, 1H) 6.95 (d, 1H) 3.2 (s, 3H) 2.3-2.2 (m, 1H) 1.15-1.10 (m, 2H) 0.8-0.75 (m, 2H)

### Example 82

### 5-(4-Methylpyridin-3-yl)thiophene-2-sulphonic acid N-methyl-N-[4-(3-phenylureido)phenyl]-amide

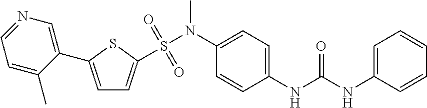

From 4-methylpyridine-3-boronic acid and 5-bromothiophene-2-sulphonic acid N-methyl-N-[4-(3-phenylureido)phenyl]amide (Example 55) using [1,1-Bis(diphenylphosphino)ferrocene]dichloropalladium (II) and aqueous Na₂CO₃ (2M) in acetonitrile.

LCMS (Method C) Rt 9.28 (M+H⁺) 479

¹H NMR (400 MHz) (DMSO-d₆) δ 8.9 (br s, 1H) 8.8 (br s, 1H) 8.5 (s, 1H) 8.4 (d, 1H) 7.5 (d, 1H) 7.45-7.4 (m, 5H) 7.35 (d, 1H) 7.2 (t, 2H) 7.0 (d, 2H) 6.9 (t, 1H) 3.2 (s, 3H) 2.4 (s, 3H).

### Example 83

### 5-(2-Methoxypyridin-3-yl)thiophene-2-sulphonic acid N-methyl-[4-(3-phenyl-ureido)-phenyl]amide

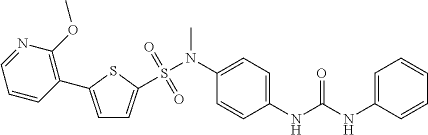

From 2-methoxypyridine-3-boronic acid and 5-bromothiophene-2-sulphonic acid N-methyl-N-[4-(3-phenylureido)phenyl]amide (Example 55) using [1,1′-Bis(diphenylphosphino)ferrocene]dichloropalladium (II) and aqueous Na₂CO₃ (2M) in acetonitrile.

LCMS (Method C) Rt 12.00 (M+H⁺) 495

¹H NMR (400 MHz) (DMSO-d₆) δ 8.9 (br s, 1H) 8.8 (br s, 1H) 8.3 (dd, 1H) 8.2 (dd, 1H) 7.8 (d, 1H) 7.45-7.35 (m, 5H) 7.2 (t, 2H) 7.1 (dd, 1H) 7.0 (d, 2H) 6.9 (t, 1H) 4.0 (s, 3H) 3.1 (s, 3H).

### Example 84

### 5-(2-Methoxypyrimidin-5-yl)thiophene-2-sulphonic acid N-methyl-N-[4-(3-phenylureido)phenyl]amide

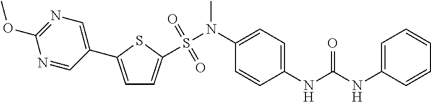

From 2-methoxypyrimidine-5-boronic acid and 5-bromothiophene-2-sulphonic acid N-methyl-N-[4-(3-phenylureido)phenyl]amide (Example 55) using [1,1-Bis(diphenylphosphino)ferrocene]dichloropalladium (II) and aqueous Na₂CO₃ (2M) in acetonitrile.

LCMS (Method C) Rt 10.95 (M+H⁺) 496

¹H NMR (400 MHz) (DMSO-d₆) δ 9.0 (s, 2H) 8.8 (br s, 1H) 8.7 (br s, 1H) 7.7 (d, 1H) 7.5 (d, 1H) 7.45 (m, 4H) 7.3 (t, 2H) 7.1 (d, 2H) 7.0 (t, 1H) 4.0 (s, 3H) 3.2 (s, 3H).

### Example 85

### 5-(2-Dimethylaminopyridin-5-yl)thiophene-2-sulphonic acid N-methyl-N-[4-(3-phenylureido)-phenyl]amide

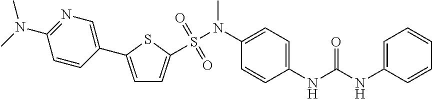

From 2-(dimethylamino)pyridine-5-boronic acid and 5-bromothiophene-2-sulphonic acid N-methyl-N-[4-(3-phenylureido)phenyl]amide (Example 55) using [1,1-Bis(diphenylphosphino)ferrocene]dichloropalladium (II) and aqueous Na₂CO₃ (2M) in acetonitrile.

LCMS (Method C) Rt 8.87 (M+H⁺) 508

¹H NMR (400 MHz) (DMSO-d₆) δ 8.7 (br s, 1H) 8.6 (br s, 1H) 8.4 (d, 1H) 7.8 (dd, 1H) 7.4 (m, 5H) 7.3 (d, 1H) 7.2 (t, 2H) 7.05 (d, 2H) 6.9 (t, 1H) 6.7 (d, 1H) 3.1 (s, 3H) 3.0 (s, 6H).

### Example 86

### 5-(Oxazol-2-yl)thiophene-2-sulphonic acid N-methyl-N-[4-(3-phenylureido)phenyl]amide

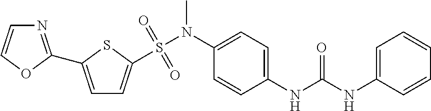

A mixture of 5-bromothiophene-2-sulphonic acid N-methyl-N-[4-(3-phenylureido)phenyl]amide (Example 55, 93 mg), 2-tributylstannyl oxazole (255 μl) and tetrakis(triphenylphosphine)palladium(0), (23 mg) in DME (3 ml) was heated in the microwave at 150° C. for 45 minutes. The reaction mixture was diluted with water and extracted with ethyl acetate. The organic layer was washed with water, dried (MgSO₄), filtered and the volatiles were removed by evaporation. The residue was purified by HPLC eluting with a mixture of water and acetonitrile (each containing 0.1% formic acid) from 50 to 70% acetonitrile over 25 minutes to give 5-(oxazol-2-yl)thiophene-2-sulphonic acid N-methyl-N-[4-(3-phenylureido)phenyl]amide (6 mg).

LCMS (Method C) Rt 10.86 (M+H⁺) 455

¹H NMR (400 MHz) (DMSO-d₆) δ 8.8 (s, 1H) 8.7 (s, 1H) 8.3 (s, 1H) 7.8 (d, 1H) 7.5 (d, 1H) 7.45 (m, 5H) 7.3 (t, 2H) 7.1 (d, 2H) 6.95 (t, 1H) 3.2 (s, 3H)

By proceeding in a similar manner the following compounds were prepared from the appropriate starting materials:

### Example 87

### 5-(1-Methyl-1H-imidazol-5-yl)thiophene-2-sulphonic acid N-methylN-[4-(3-phenylureido)-phenyl]amide

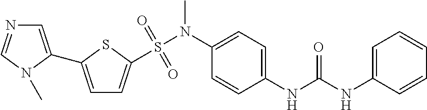

From 1-methyl-5-tributylstannyl-1H-imidazole and 5-bromothiophene-2-sulphonic acid N-methyl-N-[4-(3-phenylureido)phenyl]amide (Example 55)

LCMS (Method C) Rt 7.33 (M+H⁺) 468

¹H NMR (400 MHz) (DMSO-d₆) δ 8.9 (br s, 1H) 8.8 (br s, 1H) 7.8 (s, 1H) 7.5 (m, 5H) 7.4 (d, 1H) 7.3 (m, 3H) 7.1 (d, 2H) 7.0 (t, 1H) 3.8 (s, 3H) 3.2 (s, 3H)

### Example 88

### 5-(3-Methylpyridin-2-yl)thiophene-2-sulphonic acid N-methyl-N-[4-(3-phenylureido)-phenyl]-amide

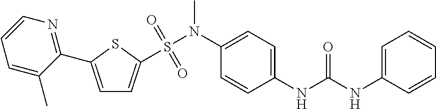

From 3-methyl-2-(tributylstannyl)pyridine and 5-bromothiophene-2-sulphonic acid N-methyl-N-[4-(3-phenylureido)phenyl]amide (Example 55)

LCMS (Method C) Rt 11.73 (M+H⁺) 479

¹H NMR (400 MHz) (DMSO-d₆) δ 8.8 (br s, 1H) 8.7 (br s, 1H) 8.4 (dd, 1H) 7.8 (dd, 1H) 7.65 (d, 1H) 7.45 (m, 5H) 7.3 (dd, 1H) 7.25 (t, 2H) 7.1 (t, 2H) 7.0 (m, 1H) 3.2 (s, 3H) 2.6 (s, 3H).

### Example 89

### 5-(1H-Pyrazol-1-yl)thiophene-2-sulphonic acid N-methyl-N-[4-(3-phenylureido)phenyl]-amide

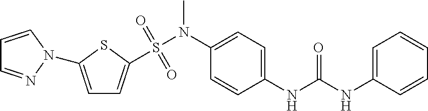

A mixture of 5-bromothiophene-2-sulphonic acid N-methyl-N-[4-(3-phenylureido)-phenyl]amide (Example 55, 140 mg), pyrazole (31 mg), cesium carbonate (196 mg), copper (I) oxide (2 mg) and salicylaldoxime (8 mg) in acetonitrile (1 ml) was heated in a sealed vial at 85° C. for 24 hours. The reaction mixture was diluted with water and extracted with ethyl acetate. The organic layer was washed with water, dried (MgSO₄), filtered and the volatiles were removed by evaporation. The residue was purified by chromatography on a 5 g silica cartridge eluting with a mixture of ethyl acetate and dichloromethane (1:39 increasing to 1:19). The resultant solid was recrystallised from a mixture of ethyl acetate and pentane to give 5-(1H-pyrazol-1-yl)thiophene-2-sulphonic acid N-methyl-N-[4-(3-phenylureido)-phenyl]amide (47 mg).

LCMS (Method C) Rt 11.11 (M+H⁺) 454

¹H NMR (400 MHz) (DMSO-d₆) δ 8.8 (br s, 1H) 8.7 (br s, 1H) 8.6 (d, 1H) 7.8 (d, 1H) 7.5-7.4 (m, 6H) 7.3 (t, 2H) 7.1 (d, 2H) 7.0 (t, 1H) 6.6 (t, 1H) 3.2 (s, 3H).

By proceeding in a similar manner the following compound was prepared from the appropriate starting materials:

### Example 90

### 5-(1H-Imidazol-1-yl)thiophene-2-sulphonic acid N-methyl-N-[4-(3-phenylureido)phenyl]-amide

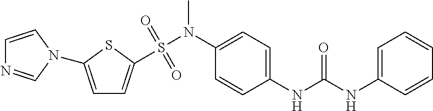

From 5-bromothiophene-2-sulphonic acid N-methyl-N-[4-(3-phenylureido)-phenyl]amide (Example 55) and imidazole.

LCMS (Method C) Rt 8.33 (M+H⁺) 453

¹H NMR (400 MHz) (DMSO-d₆) δ 9.0 (br s, 1H) 8.9 (br s, 1H) 8.2 (s, 1H) 7.7 (t, 1H) 7.45-7.4 (m, 6H) 7.2 (t, 2H) 7.1-7.05 (m, 3H) 6.9 (t, 1H) 3.15 (s, 3H).

### Example 91

### 4′-Fluorobiphenyl-3-sulphonic acid N-(3-hydroxypropyl)-N-[4-(3-phenylureido)phenyl]amide

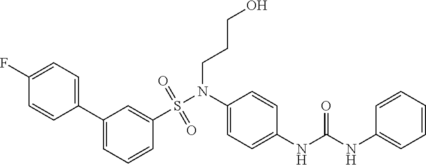

A solution of 4′-fluorobiphenyl-3-sulphonic acid N-(3-benzyloxypropyl)-N-[4-(3-phenylureido)phenyl]amide (Intermediate 43, 151 mg), palladium on carbon (10%, 29 mg) and acetic acid (128 □l) in IMS (2.5 ml) was hydrogenated under a balloon of hydrogen at atmospheric pressure overnight. The catalyst was removed by filtration through Celite under nitrogen and the filtrate was concentrated to dryness. The residue was purified by HPLC eluting with a mixture of water and acetonitrile (each containing 0.1% formic acid) from 20 to 95% acetonitrile over 25 minutes to give 4′-fluorobiphenyl-3-sulphonic acid N-(3-hydroxypropyl)-N-[4-(3-phenylureido)phenyl]amide (35 mg).

LCMS (Method C) Rt 11.28 (M+H⁺) 520

¹H NMR (400 MHz) ((DMSO-d₆) δ 8.8 (s, 1H) 8.7 (s, 1H), 8.0 (d, 1H) 7.7 (m, 4H) 7.6 (d, 1H) 7.5 (m, 4H) 7.3 (m, 4H) 7.0 (m, 3H) 4.4 (t, 1H) 3.6 (t, 2H) 3.4 (m, 2H) 1.5 (m, 2H)

### Example 92

### 1-(4-Fluorophenyl)-3,5-dimethyl-1H-pyrazole-4-sulphonic acid N-(3-hydroxypropyl)-N-[4-(3-phenylureido)phenyl]amide

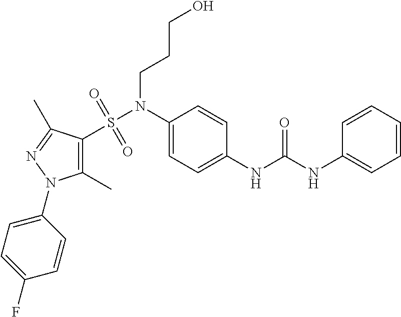

### STEP 1:1-(4-Fluorophenyl)-3,5-dimethyl-1H-pyrazole-4-sulphonic acid N-(3-benzyl-oxypropyl)-N-[4-(3-phenylureido)phenyl]amide

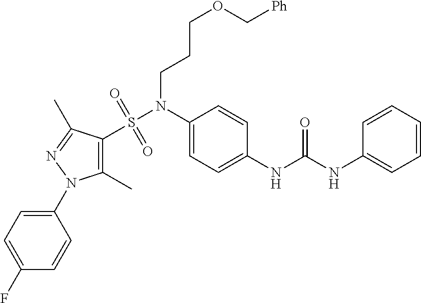

1-(4-Fluorophenyl)-3,5-dimethyl-1H-pyrazole-4-sulphonic acid [4-(3-phenylureido)phenyl]-amide (Example 24, 202 mg) was dissolved in THF (6.0 ml). Triphenylphosphine (221 mg) was added followed by 3-benzyloxy-1-propanol (133 μl). The mixture was cooled to 0° C. and diethyl azodicarboxylate (132 μl) was slowly added and the reaction mixture was stirred overnight at room temperature. The volatiles were removed by evaporation and the residue was purified by chromatography using the Biotage system on a 10 g silica cartridge eluting with a mixture of ethyl acetate and cyclohexane (1:19 increasing to 3:7) to give 1-(4-fluorophenyl)-3,5-dimethyl-1H-pyrazole-4-sulphonic acid N-(3-benzyloxypropyl)-N-[4-(3-phenylureido)phenyl]amide (339 mg).

LCMS (Method A) Rt 4.23 (M+H⁺) 628

### STEP 2: 1-(4-Fluorophenyl)-3,5-dimethyl-1H-pyrazole-4-sulphonic acid N-(3-hydroxypropyl)-N-[4-(3-phenylureido)phenyl]amide

The deprotection step was carried out in a similar manner to that used for Example 91.

LCMS (Method C) Rt 10.20 (M+H⁺) 538

¹H NMR (400 MHz) ((DMSO-d₆) δ 8.8 (s, 1H) 8.7 (s, 1H) 7.6 (m, 2H) 7.4 (m, 6H) 7.3 (t, 2H) 7.1 (d, 2H) 7.0 (t, 1H) 4.4 (t, 1H) 3.6 (t, 2H) 3.4 (m, 2H) 2.1 (s, 3H) 2.0 (s, 3H) 1.5 (m, 2H)

### Example 93

### 5-(Pyridin-2-yl)thiophene-2-sulphonic acid N-(3-hydroxypropyl)-N-[4-(3-phenylureido)-phenyl]amide

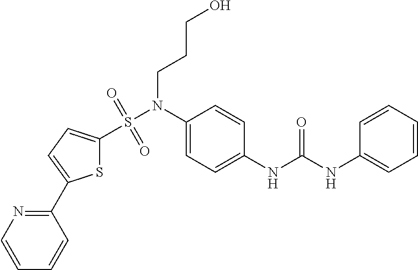

### STEP1: 5-(Pyridin-2-yl)thiophene-2-sulphonic acid N-(3-tert-butoxypropyl)-N-[4-(3-phenylureido)phenyl]amide

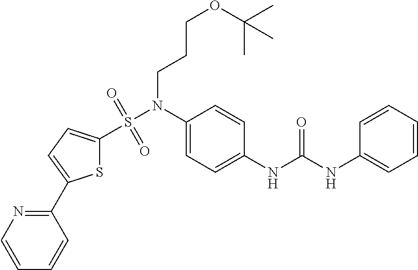

The alkylation was performed in a similar manner to that used in Example 92, Step 1 starting from 5-(pyridin-2-yl)thiophene-2-sulphonic acid [4-(3-phenylureido)phenyl]amide (Example 32) and 3-tert-butoxy-1-propanol

LCMS (Method A) Rt 4.16 (M+H⁺) 565

### STEP2: 5-(Pyridin-2-yl)thiophene-2-sulphonic acid N-(3-hydroxypropyl)-N-[4-(3-phenyl-ureido)phenyl]amide

To an ice-cooled solution of 5-(pyridin-2-yl)thiophene-2-sulphonic acid N-(3-tert-butoxypropyl)-N-[4-(3-phenylureido)phenyl]amide (from step 1, 425 mg) in DCM (9 ml) and MeOH (1 ml) was added TFA (5 ml). The resultant mixture was stirred at room temperature overnight. The volatiles were removed by evaporation and the residue was dissolved in THF (10 ml) and treated with NaOH (1M, 5 ml). The resultant mixture was stirred at room temperature for 1 hour. The volatiles were removed by evaporation and the residue was purified by HPLC eluting with a mixture of water and acetonitrile (each containing 0.1% formic acid) from 50 to 70% acetonitrile over 20 minutes to give 5-(pyridin-2-yl)thiophene-2-sulphonic acid N-(3-hydroxypropyl)-N-[4-(3-phenylureido)phenyl]amide (325 mg).

LCMS (Method C) Rt 10.03 (M+H⁺) 509

¹H NMR (400 MHz) ((DMSO-d₆) δ 8.8 (s, 1H) 8.7 (s, 1H) 8.6 (d, 1H) 8.1 (d, 1H) 7.9 (m, 2H) 7.5 (m, 6H) 7.3 (t, 2H) 7.0 (d, 2H) 6.9 (t, 1H) 4.5 (t, 1H) 3.7 (t, 2H) 3.4 (dt, 2H) 1.3 (t, 2H)

### Example 94

### 4′-Fluorobiphenyl-3-sulphonic acid N-(3-hydroxypropyl)-N-[2-methoxy-4-(3-phenylureido)-phenyl]amide

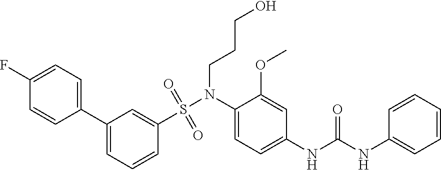

A solution of 4′-fluorobiphenyl-3-sulphonic acid N-(3-tert-butoxypropyl)-N-[2-methoxy-4-(3-phenylureido)phenyl]amide (Intermediate 38, 110 mg) in DCM (2 ml) was treated with TFA (1 ml). The resultant mixture was stirred at room temperature for 2 hours, then diluted with DCM and washed with water. The organic layer was dried (MgSO₄), filtered and the volatiles were removed by evaporation. The residue was dissolved in MeOH (5 ml) and treated with HCl (1M, 1.7 ml). The resultant mixture was stirred at room temperature for 2 hours, concentrated and partitioned between water and ethyl acetate. The aqueous layer was further extracted with ethyl acetate and the combined organic layers were washed with water, dried (MgSO₄) and filtered. The volatiles were removed by evaporation to give 4′-fluorobiphenyl-3-sulphonic acid N-(3-hydroxypropyl)-N-[2-methoxy-4-(3-phenylureido)-phenyl]amide as an off-white solid (74 mg).

LCMS (Method C) Rt 11.35 (M+H⁺) 550

¹H NMR (400 MHz) ((DMSO-d₆) δ 8.8 (s, 1H) 8.7 (s, 1H) 7.9 (dd, 1H) 7.7 (m, 5H) 7.5 (d, 2H) 7.3 (m, 5H) 7.1 (d, 1H) 7.0 (t, 1H) 6.9 (dd, 1H) 4.2 (br s, 1H) 3.6 (br s, 2H) 3.4 (t, 2H) 3.3 (s, 3H) 1.5 (m, 2H)

### Example 95

### 1-(4-Fluorophenyl)-1H-pyrazole-4-sulphonic acid N-(3-hydroxypropyl)-N-[4-(3-phenyl-ureido)phenyl]amide

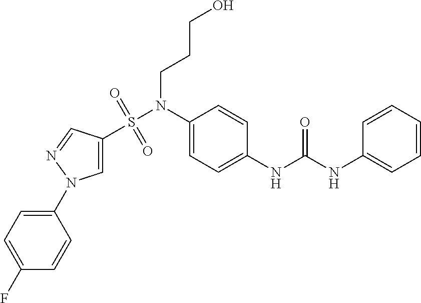

### STEP1: 1-(4-Fluorophenyl)-1H-pyrazole-4-sulphonic acid N-(3-tertbutoxypropyl)-N-[4-(3-phenylureido)phenyl]amide

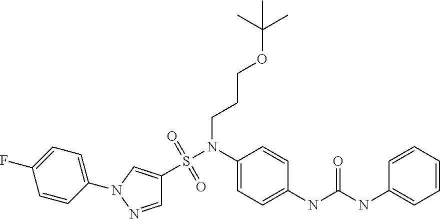

The alkylation was performed in a similar manner to that used for Example 92, Step1 starting from 1-(4-fluorophenyl)-1H-pyrazole-4-sulphonic acid [4-(3-phenylureido)phenyl]amide (Intermediate 8) and 3-tert-butoxy-1-propanol

LCMS (Method A) Rt 4.15 (M+H⁺) 566

### STEP2: 1-(4-Fluorophenyl)-1H-pyrazole-4-sulphonic acid N-(3-hydroxypropyl)-N-[4-(3-phenylureido)phenyl]amide

The deprotection step was carried out in a similar manner to that used for Example 94

LCMS (Method C) Rt 10.33 (M+H⁺) 510

¹H NMR (400 MHz) ((DMSO-d₆) δ 9.0 (s, 1H) 8.8 (s, 1H) 8.7 (s, 1H) 8.0 (m, 2H), 7.9 (s, 1H) 7.4 (m, 6H) 7.3 (t, 2H) 7.1 (d, 2H) 7.0 (t, 1H) 4.4 (t, 1H) 3.6 (t, 2H) 3.4 (m, 2H) 1.5 (m, 2H)

### Example 96

### 5-(1-Methyl-3-trifluoromethyl-1H-pyrazol-5-yl)thiophene-2-sulphonic acid N-(3-hydroxy-propyl)-N-[4-(3-phenylureido)phenyl]amide

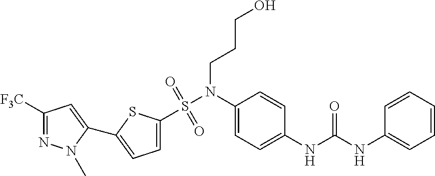

### STEP 1: 5-(1-Methyl-3-trifluoromethyl-1H-pyrazol-5-yl)thiophene-2-sulphonic acid N-(3-tert-butoxypropyl)-N-[4-(3-phenylureido)phenyl]amide

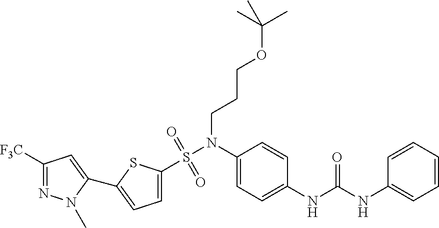

The alkylation step was carried out in a similar manner to that used for Example 92, Step1 starting from 5-(1-methyl-3-trifluoromethyl-1H-pyrazol-5-yl)thiophene-2-sulphonic acid [4-(3-phenylureido)phenyl]amide (Example 33) and 3-tert-butoxypropan-1-ol

LCMS (Method B) Rt 4.48 (M+H⁺) 636

### STEP 2: 5-(1-Methyl-3-trifluoromethyl-1H-pyrazol-5-yl)thiophene-2-sulphonic acid N-(3-hydroxypropyl)-N-[4-(3-phenylureido)phenyl]amide

The deprotection was carried out in a similar manner to that used for Example 94, Step 2.

LCMS (Method C) Rt 11.14 (M+H⁺) 580

¹H NMR (400 MHz) ((DMSO-d₆) δ 8.9 (s, 1H) 8.7 (s, 1H) 7.7 (d, 1H) 7.6 (d, 1H) 7.5 (m, 4H) 7.3 (t, 2H) 7.2 (s, 1H) 7.1 (d, 2H) 7.0 (t, 1H) 4.5 (t, 1H) 4.1 (s, 3H) 3.6 (t, 2H) 3.4 (m, 2H) 1.5 (m, 2H)

### Example 97

### 5-(1H-Pyrazol-5-yl)thiophene-2-sulphonic acid N-methyl-N-[4-(3-phenylureido)phenyl]-amide

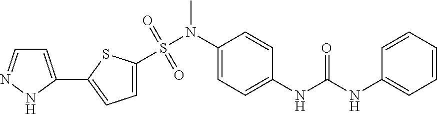

To a suspension of 5-[1-(tetrahydropyran-2-yl)-1H-pyrazol-5-yl]-thiophene-2-sulphonic acid N-methyl-N-[4-(3-phenylureido)phenyl]amide (Intermediate 33, 62 mg) in MeOH (1 ml) was added a solution of HCl in MeOH (1.25 M, 2.0 ml). The mixture was stirred for 1 h, then diluted with water and treated with saturated aqueous NaHCO₃. The resultant mixture was extracted with ethyl acetate and the organic layer was washed with water, dried (MgSO₄), filtered and the volatiles were removed by evaporation. The residue was purified by HPLC eluting with a mixture of water and acetonitrile (each containing 0.1% formic acid) from 50 to 70% acetonitrile over 20 minutes to give 5-(1H-pyrazol-5-yl)thiophene-2-sulphonic acid N-methyl-N-[4-(3-phenylureido)phenyl]amide (24 mg).

LCMS (Method C) Rt 10.10 (M+H⁺) 454

¹H NMR (400 MHz) ((DMSO-d₆) δ 13.1 (br s, 1H) 8.8 (s, 1H) 8.7 (s, 1H) 7.8 (s, 1H) 7.4 (m, 6H) 7.3 (t, 2H) 7.1 (d, 2H) 7.0 (t, 1H) 6.8 (s, 1H) 3.2 (s, 3H)

### Example 98

### 4′-Fluorobiphenyl-3-sulphonic acid N-(3-dimethylaminopropyl)-N-[4-(3-phenylureido)-phenyl]amide

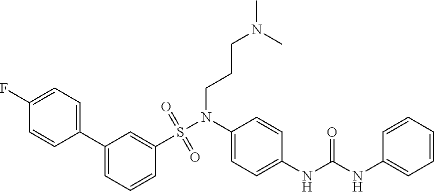

### STEP 1: 4′-Fluorobiphenyl-3-sulphonic acid N-(3-methanesulphonyloxypropyl)-N-[4-(3-phenylureido)phenyl]amide

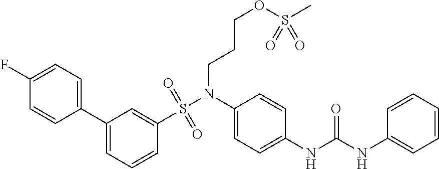

To an ice-cooled solution of 4′-fluorobiphenyl-3-sulphonic acid N-(3-hydroxypropyl)-N-[4-(3-phenylureido)phenyl]amide (Example 91, 100 mg) and triethylamine (30 μl) in DCM (3 ml) was added methanesulphonyl chloride (17 μl). The mixture was stirred at room temperature for 3 hours and then further methanesulphonyl chloride was added (10 μl). After a further 1 hour, the reaction mixture was poured into a mixture of ice and water, and extracted with DCM. The organic layer was dried (Na₂SO₄), filtered and the volatiles were removed by evaporation to give 4′-fluorobiphenyl-3-sulphonic acid N-(3-methanesulphonyloxypropyl)-N-[4-(3-phenylureido)phenyl]amide (84 mg).

LCMS (Method A) Rt. 3.95 (M+H⁺) 598

### STEP 2: 4′-Fluorobiphenyl-3-sulphonic acid N-(3-dimethylaminopropyl)-N-[4-(3-phenylureido)phenyl]amide

A mixture of 4′-fluorobiphenyl-3-sulphonic acid N-(3-methanesulphonyloxypropyl)-N-[4-(3-phenylureido)phenyl]amide (from Step 1, 68 mg) and dimethylamine (40% in water, 86 μl) in 1,4-dioxane (230 μl) was heated in the microwave at 125° C. for 5 minutes. The resultant mixture was concentrated and the residue was purified by HPLC eluting with a mixture of water and acetonitrile (each containing 0.1% formic acid) from 20 to 98% acetonitrile over 25 minutes to give 4′-fluorobiphenyl-3-sulphonic acid N-(3-dimethylaminopropyl)-N-[4-(3-phenylureido)phenyl]amide (12 mg).

LCMS (Method C) Rt 8.41 (M+H⁺) 547

¹H NMR (400 MHz) ((DMSO-d₆) δ 9.2 (s, 1H) 9.1 (s, 1H) 8.2 (s, 1H) 8.0 (d, 1H) 7.7 (m, 4H) 7.6 (d, 1H) 7.5 (m, 3H) 7.3 (m, 4H) 7.0 (m, 3H) 3.6 (t, 2H) 2.3 (t, 2H) 2.1 (s, 6H) 1.5 (m, 2H)

### Example 99

### 4′-Fluorobiphenyl-3-sulphonic acid N-(2,3-dihydroxypropyl)-N-[4-(3-phenylureido)phenyl]-amide

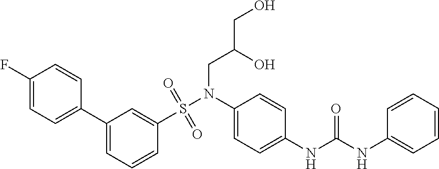

A mixture of 4′-fluorobiphenyl-3-sulphonic acid N-[(2,2-dimethyl-1,3-dioxolan-4-yl)methyl]-N-[4-(3-phenylureido)phenyl]amide (Intermediate 45, 57 mg) in acetone (2 ml) containing HCl (1M, 1 ml) was heated at reflux for 4 hours. The mixture was concentrated and the residue was purified by HPLC eluting with a mixture of water and acetonitrile (each containing 0.1% formic acid) from 20 to 98% acetonitrile over 30 minutes to give 4′-fluorobiphenyl-3-sulphonic acid N-(2,3-dihydroxypropyl)-N-[4-(3-phenylureido)phenyl]-amide (23 mg).

LCMS (Method C) Rt 10.40 (M+H⁺) 536

¹H NMR (400 MHz) ((DMSO-d₆) δ 8.9 (s, 1H) 8.8 (s, 1H) 8.0 (d, 1H) 7.7 (m, 4H) 7.6 (d, 1H) 7.4 (m, 4H) 7.3 (m, 4H) 7.0 (m, 3H) 4.8 (d, 1H) 4.5 (t, 1H) 3.5 (m, 2H) 3.4-3.2 (m, 3H)

### Example 100

### 5-Cyclohexylthiophene-2-sulphonic acid N-methyl-N-[4-(3-phenylureido)phenyl]amide

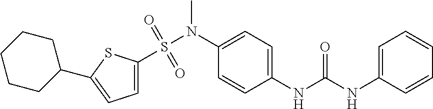

5-(Cyclohexen-1-yl)thiopheny-2-sulphonic acid N-methyl-N-[4-(3-phenylureido)phenyl]-amide (Example 74, 32 mg) was dissolved in a mixture of ethyl acetate (2 ml) and THF (1 ml) and treated with palladium on carbon (10%, 65 mg). The reaction mixture was hydrogenated under a balloon of hydrogen at atmospheric pressure for 4 hours. The catalyst was removed by filtration through Celite under nitrogen and the filtrate was concentrated to dryness to give 5-cyclohexylthiophene-2-sulphonic acid N-methyl-N-[4-(3-phenylureido)phenyl]amide (20 mg) as a white solid.

LCMS (Method C) Rt 13.57 (M+H⁺) 470

¹H NMR (400 MHz) (DMSO-d₆) δ 9.2 (s, 1H) 9.1 (s, 1H) 7.5 (m, 4H) 7.3 (m, 3H) 7.0 (m, 4H) 3.2 (s, 3H) 2.9 (m, 1H) 2.0 (m, 2H) 1.75 (m, 2H) 1.6 (m, 1H) 1.4 (m, 4H) 1.3 (m, 1H)

### Example 101

### 5-(1,2,3,6-Tetrahydropyridin-4-yl)thiophene-2-sulphonic acid N-methyl-N-[4-(3-phenyl-ureido)phenyl]amide

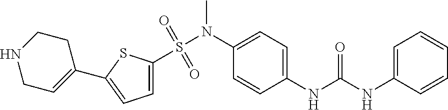

5-(1-Boc-3,6-dihydro-2H-pyridin-4-yl)thiophene-2-sulphonic acid N-methyl-N-[4-(3-phenyl-ureido)phenyl]amide (Intermediate 35, 45 mg) was dissolved in dichloromethane (1 ml) and treated with TFA (1 ml). The mixture was stirred at room temperature for 1 hour then the volatiles were removed by evaporation. The residue was purified by passing through an SCX-2 column eluting with DCM followed by a mixture of DCM and methanol (1:1) and finally a mixture of DCM and 2M ammonia in methanol (1:1). After evaporation of the volatiles, the residue was purified by HPLC eluting with a mixture of water and acetonitrile (each containing 0.1% formic acid) from 20 to 98% acetonitrile to give 5-(1,2,3,6-tetrahydropyridin-4-yl)thiophene-2-sulphonic acid N-methyl-N-[4-(3-phenylureido)phenyl]-amide (20 mg).

LCMS (Method C) Rt 7.20 (M+H⁺) 469

¹H NMR (400 MHz) (DMSO-d₆) δ 9.5 (s, 1H) 9.4 (s, 1H) 7.5-7.4 (m, 4H) 7.3 (d, 1H) 7.2 (t, 2H) 7.16 (d, 1H) 6.3 (br s, 1H) 3.9-3.2 (broad signal) 3.1 (s, 3H) 3.0 (broad signal), 2.4 (broad signal).

### Example 102

### 5-(1-Methylpiperidin-4-yl)thiophene-2-sulphonic acid N-methyl-N-[4-(3-phenylureido)-phenyl]amide

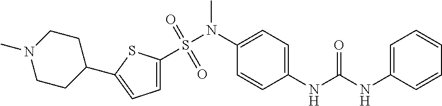

### STEP 1: 5-(1-Boc-piperidin-4-yl)thiophene-2-sulphonic acid N-methyl-N-[4-(3-phenylureido)phenyl]amide

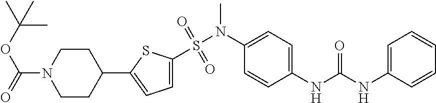

Prepared by proceeding in a similar manner to that used for Example 100 starting from 5-(1-Boc-3,6-dihydro-2H-pyridin-4-yl)thiophene-2-sulphonic acid N-methyl-N-[4-(3-phenyl-ureido)phenyl]amide (Intermediate 35).

LCMS (Method B) Rt 4.20 (M+H⁺) 571

### STEP 2: 5-(Piperidin-4-yl)thiophene-2-sulphonic acid N-methyl-N-[4-(3-phenylureido)-phenyl]amide

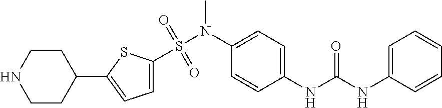

The deprotection was carried out in a similar manner to that used for Example 101.

LCMS (Method C) Rt 7.15 (M+H⁺) 471

¹H NMR (400 MHz) (DMSO-d₆) 8.85 (br s, 1H) 8.75 (br s, 1H) 7.45 (t, 4H) 7.3 (m, 3H) 7.0 (m, 3H) 6.95 (t, 1H) 3.15 (s, 3H) 3.0 (m, 3H) 2.55 (dt, 2H) 1.85 (m, 2H) 1.45 (dq, 2H).

### STEP 3: 5-(1-Methylpiperidin-4-yl)thiophene-2-sulphonic acid N-methyl-N-[4-(3-phenyl-ureido)phenyl]amide

A mixture of 5-(piperidin-4-yl)thiophene-2-sulphonic acid N-methylN-[4-(3-phenylureido)-phenyl]amide (from Step 2, 235 mg), formaldehyde (60 μl), acetic acid (60 μl), and sodium triacetoxyborohydride (60 mg) in dichloromethane (5 ml) was stirred at room temperature under nitrogen for 18 hours. The mixture was quenched with water and extracted with DCM and then with ethyl acetate. The combined organic layers were dried (Na₂SO₄), filtered and the volatiles were removed by evaporation. The residue was purified by chromatography using a 5 g silica cartridge eluting with DCM followed by a mixture of DCM and 2M ammonia in methanol (9:1) to give 5-(1-methylpiperidin-4-yl)thiophene-2-sulphonic acid N-methyl-N-[4-(3-phenylureido)phenyl]amide as a white solid (32 mg)

LCMS (Method C) Rt 7.20 (M+H⁺) 485

¹H NMR (400 MHz) (DMSO-d₆) δ 8.8 (br s, 1H) 8.7 (br s, 1H) 7.45 (m, 4H) 7.3 (m, 3H) 7.0 (m, 3H) 6.95 (t, 1H) 3.1 (s, 3H) 2.8 (m, 3H) 2.1 (s, 3H) 1.95 (m, 4H) 1.6 (m, 2H).

### Example 103

### 5-(3-Trifluoromethyl-1H-pyrazol-5-yl)thiophene-2-sulphonic acid N-methyl-N-[4-(3-phenyl-ureido)phenyl]amide

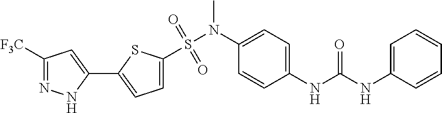

5-(4,4,4-Trifluoro-1,3-dioxobutyl)thiophene-2-sulphonic acid N-methyl-N-[4-(3-phenyl-ureido)phenyl]amide (Intermediate 9, 34 mg) was dissolved in IMS (1 ml) and hydrazine hydrate (30 μl) was added. The mixture was stirred at room temperature for 1 hour then heated at 70° C. for 3 hours and allowed to stand at room temperature overnight. The reaction was repeated using 5-(4,4,4-trifluoro-1,3-dioxobutyl)thiophene-2-sulphonic acid N-methyl-N-[4-(3-phenylureido)phenyl]amide (Intermediate 9, 96 mg), IMS (3 ml) and hydrazine hydrate (83 μl). The two crude reaction mixtures were combined and poured into water and extracted with ethyl acetate. The organic layer was washed with water, dried (MgSO₄), filtered and the volatiles were removed by evaporation. The residue was purified by HPLC eluting with a mixture of water and acetonitrile (each containing 0.1% formic acid) from 50 to 70% acetonitrile over 20 minutes to give 5-(3-trifluoromethyl-1H-pyrazol-5-yl)thiophene-2-sulphonic acid N-methyl-N-[4-(3-phenylureido)phenyl]amide (11 mg).

LCMS (Method C) Rt 11.62 (M+H⁺) 522

¹H NMR (400 MHz) ((DMSO-d₆) δ 14.5 (br s, 1H) 8.9 (s, 1H) 8.8 (s, 1H) 7.65 (d, 1H) 7.55 (d, 1H) 7.5 (m, 4H) 7.3 (m, 3H) 7.1 (d, 2H) 7.0 (t, 1H) 3.2 (s, 3H)

### Example 104

### 1-(4-Fluorophenyl)-1H-imidazole-4-sulphonic acid N-methyl-N-[4-(3-phenylureido)phenyl]-amide

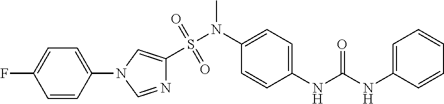

A mixture of 4-fluorophenylboronic acid (160 mg), copper acetate (65 mg) and 1H-imidazole-4-sulphonic acid N-methyl-N-[4-(3-phenylureido)phenyl]amide (Intermediate 16, 98 mg) in pyridine (2 ml) containing 4 A Molecular Sieves was stirred at room temperature for 18 hours and then heated at 40° C. further 18 hours. The mixture was filtered through Celite and the filtrate was diluted with water and extracted with DCM. The organic layer was dried (Na₂SO₄), filtered and the volatiles were removed by evaporation. The residue was purified by chromatography using a 5 g silica cartridge eluting with DCM and then a mixture of ethyl acetate and DCM (1:9 to 1:4). The resultant product was further purified by HPLC eluting with a mixture of water and acetonitrile (each containing 0.1% formic acid) from 40 to 60% acetonitrile over 20 minutes to give 1-(4-fluorophenyl)-1H-imidazole-4-sulphonic acid N-methyl-N-[4-(3-phenylureido)phenyl]amide as a white solid (28 mg).

LCMS (Method C) Rt 10.49 (M+H⁺) 466

¹H NMR (400 MHz) (DMSO-d₆) δ 8.7 (br s, 1H) 8.6 (br s, 1H) 8.4 (d, 1H) 8.2 (d, 1H) 7.8-7.7 (m, 2H) 7.4-7.3 (m, 6H) 7.2 (m, 2H) 7.1 (d, 2H) 6.9 (t, 1H) 3.2 (s, 3H).

By proceeding in a similar manner the following compounds were prepared from the appropriate starting materials:

### Example 105

### 1-Phenyl-1H-pyrazole-4-sulphonic acid N-methyl-N-[4-(3-phenylureido)phenyl]amide

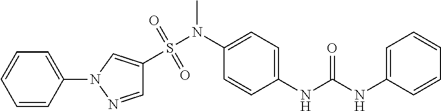

From phenylboronic acid and 1H-pyrazole-4-sulphonic acid N-methyl-N-[4-(3-phenylureido)-phenyl]amide (Intermediate 17)

LCMS (Method C) Rt 11.36 (M+H⁺) 448

¹H NMR (400 MHz) (DMSO-d₆) δ 9.0 (s, 1H) 8.8 (s, 1H) 8.7 (s, 1H) 7.9 (d, 2H) 7.8 (s, 1H) 7.5 (t, 2H) 7.4 (m, 5H) 7.3 (t, 2H) 7.2 (d, 2H) 6.9 (t, 1H) 3.1 (s, 3H).

### Example 106

### 1-(1-Methyl-1H-pyrazolyl-4-yl)-1H-pyrazole-4-sulphonic acid N-methyl-N-[4-(3-phenyl-ureido)phenyl]amide

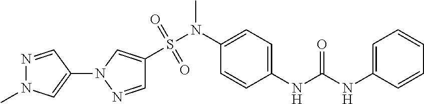

From 1-methyl-1-H-pyrazole-4-boronic acid and 1H-pyrazole-4-sulphonic acid N-methyl-N-[4-(3-phenyl ureido)phenyl]amide (Intermediate 17)

LCMS (Method C) Rt 9.51 (M+H⁺) 452

¹H NMR (400 MHz) (DMSO-d₆) δ 9.0 (br s, 1H) 8.9 (br s, 1H) 8.7 (s, 1H) 8.3 (s, 1H) 8.0 (s, 1H) 7.8 (s, 1H) 7.5 (t, 4H) 7.3 (t, 2H) 7.1 (d, 2H) 7.0 (t, 1H) 3.9 (s, 3H) 3.2 (s, 3H).

### Example 107

### 1-(2-Methoxypyridin-5-yl)-1H-pyrazole-4-sulphonic acid N-methyl-N-[4-(3-phenylureido)-phenyl]amide

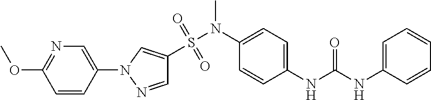

From 2-methoxy-5-pyridineboronic acid and 1H-pyrazole-4-sulphonic acid N-methyl-N-[4-(3-phenylureido)phenyl]amide (Intermediate 17)

LCMS (Method C) Rt 10.98 (M+H⁺) 479

¹H NMR (400 MHz) (DMSO-d₆) 9.0 (s, 1H) 8.8 (s, 1H) 8.7 (d, 1H) 8.65 (s, 1H) 8.2 (dd, 1H) 7.8 (s, 1H) 7.45 (m, 4H) 7.3 (m, 2H) 7.1 (m, 2H) 7.0 (m, 2H) 3.9 (s, 3H) 3.2 (s, 3H).

1. A sulphonamide derivative of formula (I) or (I′) or a physiologically acceptable salt thereof, 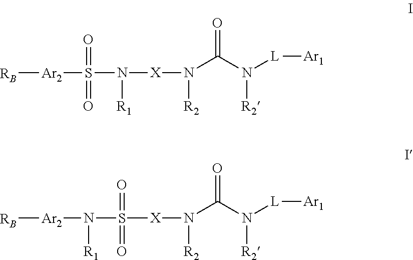
where
R₁ is H, C₁₋₆-alkyl optionally substituted with one or two hydroxyl groups, C₂₋₆-alkenyl optionally substituted with one or two hydroxyl groups, R′R″N—C₁₋₆-alkyl-, C₁₋₆-alkanoyl, R′OOC—C₁₋₆-alkyl-, R′OOC—C₁₋₆-alkoxy- or C₁₋₆-alkoxy-C₁₋₆-alkyl-;
R₂ and R_{2′} are independently selected from H and C₁₋₆-alkyl;
L is absent or a linker, which is a linear or a branched hydrocarbon chain with 1-6 carbon atoms;
X is a 5- or 6-membered aromatic ring with 0-2 heteroatoms selected from N, O and S and optionally substituted with R₃;
R₃ is OH, C₁₋₆-alkyl optionally substituted with one or two hydroxyl groups, C₂₋₆-alkenyl optionally substituted with one or two hydroxyl groups, halo-C₁₋₆-alkyl, halo-C₁₋₆-alkoxy, cyclo-C₃₋₆-alkyl, C₁₋₆-alkoxy, C₁₋₆-alkanoyl, R′OOC—C₁₋₆-alkyl-, R′OOC—C₁₋₆-alkoxy-, —NO₂, —CN, NC—C₁₋₆-alkyl-, halogen, R″R′N—C₁₋₆-alkyl-, R″R′N—C₁₋₆-alkoxy-, R″—C(O)—NR′—C₁₋₆-alkyl-, R″R′N—C(O)—C₁₋₆-alkyl, R″—C(O)—NR′—C₁₋₆-alkoxy-, R″R′N—C(O)—C₁₋₆-alkoxy, —NR′R″, —NR′—C(O)—R″, —C(O)—NHR′, C₁₋₆-alkoxy-C₁₋₆-alkyl- or C₁₋₆-alkoxy-C₁₋₆-alkoxy-;
alternatively R₂ and R₃ form together a moiety selected from the group consisting of: 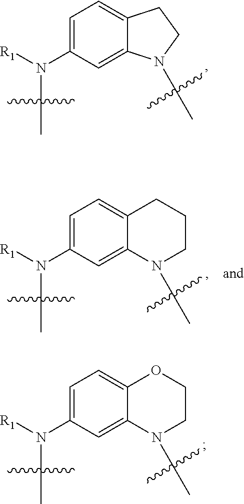
Ar₁ is a 5- or 6-membered saturated or unsaturated ring with 0 to 2 heteroatoms selected independently from N, O and S and optionally substituted with one or more groups selected from C₁₋₆-alkyl optionally substituted with one or two hydroxyl groups, C₂₋₆-alkenyl optionally substituted with one or two hydroxyl groups, halo-C₁₋₆-alkyl, cyclo-C₃₋₆-alkyl, C₁₋₆-alkoxy, halo-C₁₋₆-alkoxy, C₁₋₆-alkanoyl, R′OOC—C₁₋₆-alkyl, R′OOC—C₁₋₆-alkoxy-, —NO₂, —CN, NC—C₁₋₆-alkyl-, halogen, R″R′N—C₁₋₆-alkyl-, R″R′N—C₁₋₆-alkoxy-, R″—C(O)—NR′—C₁₋₆-alkyl-, R″R′N—C(O)—C₁₋₆-alkyl-, R″-C(O)—NR′—C₁₋₆-alkoxy-, R″R′N—C(O)—C₁₋₆-alkoxy, —NR′R″, —NR′—C(O)—R″, —C(O)—NR″R′, C₁₋₆-alkoxy-C₁₋₆-alkyl- and C₁₋₆-alkoxyC₁₋₆-alkoxy-;
Ar₂ is a ring or a fused ring system, in which the ring or the ring system is unsaturated or saturated, includes 5-12 atoms of which 0-4 are heteroatoms selected from N, O, and S, and is optionally substituted with one or more groups selected from C₁₋₆-alkyl optionally substituted with one or more hydroxyl groups, C₂₋₆-alkenyl optionally substituted with one or two hydroxyl groups, C₁₋₆-alkanoyl, C₁₋₆-alkoxy, C₁₋₆-alkoxy-C₁₋₆-alkyl- and halogen;
R_{B} is a 3-membered hydrocarbon ring or a 4-, 5-, or 6-membered saturated or unsaturated ring with 0 to 3 heteroatoms independently selected from N, O and S and optionally substituted with one or more groups selected from C₁₋₆-alkyl optionally substituted with one or two hydroxyl groups, C₂₋₆-alkenyl optionally substituted with one or two hydroxyl groups, halo-C₁₋₆-alkyl, cyklo-C₃₋₆-alkyl, C₁₋₆-alkoxy, C₁₋₆-alkanoyl, R′OOC—C₁₋₆-alkyl-, R′OOC—C₁₋₆-alkoxy-, R″R′N—C₁₋₆-alkyl-, R″R′N—C₁₋₆-alkoxy-, —NR′R″, pyrrolidyl and halogen;
alternatively R_{B} is selected from H, C₁₋₆-alkyl optionally substituted with one or two hydroxyl groups, C₂₋₆-alkenyl optionally substituted with one or two hydroxyl groups, halo-C₁₋₆-alkyl, halogen, halo-C₁₋₆-alkoxy, —NR′R″, C₁₋₆-alkoxy and —CN;
R′ and R″ are independently selected from H, C₁₋₆-alkyl optionally substituted with one or more hydroxyl groups, C₂₋₆-alkenyl optionally substituted with one or two hydroxyl groups, halo-C₁₋₆-alkyl, C₁₋₆-alkanoyl and C₁₋₆-alkoxy-C₁₋₆-alkyl;
provided that
(i) the sulphonamide derivative is not a compound of formula (I) where (a) X is methoxy-substituted phenyl and Ar₂ is pentafluorophenyl, or (b) R₁ is hydrogen and Ar₁ is substituted phenyl;
(ii) the sulphonamide derivative is not a compound of formula (I′), where L is —CH₂— and Ar₁ is phenyl; and
(iii) the sulphonamide derivative is not a compound of formula (I′) wherein L is absent, X is Cl— or methyl-phenyl, Ar₁ is CN-phenyl, Ar₂ is phenyl, R₁ is ethyl and R_{B}=R₂=R₂′=H.

2. The sulphonamide derivative according to claim 1, or a physiologically acceptable salt thereof, wherein X is selected from the group consisting of phenyl, pyrrolyl, furanyl, thiophenyl, pyridinyl and pyrimidinyl.

3. The sulphonamide derivative according to claim 1, or a physiologically acceptable salt thereof, wherein the sulphonamide derivate has the general formula Ia or Ia′ 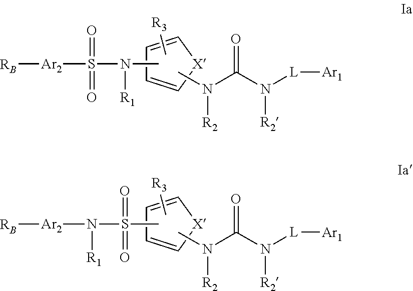
wherein x′ is selected from —CH═CH—, —CH═N— and NR′.

4. The sulphonamide derivative according to claim 1, or a physiologically acceptable salt thereof, wherein Ar₁ is phenyl optionally substituted with one or more groups selected from C₁₋₆-alkyl optionally substituted with one or two hydroxyl groups, halo-C₁₋₆-alkyl, cyclo-C₃₋₆-alkyl, C₁₋₆-alkoxy, halo-C₁₋₆-alkoxy, C₁₋₆-alkanoyl, R′OOC—C₁₋₆-alkyl-, R′OOC—C₁₋₆-alkoxy-, —NO₂, —CN, NC—C₁₋₆-alkyl- and halogen.

5. The sulphonamide derivative according to claim 1, or a physiologically acceptable salt thereof, wherein Ar₂ is an optionally substituted thiophene, pyrazolyl or phenyl.

6. The sulphonamide derivative according to claim 1, or a physiologically acceptable salt thereof, wherein R₁ is H, CH₃, hydroxyethyl or hydroxypropyl.

7. The sulphonamide derivative according to claim 3, or a physiologically acceptable salt thereof, wherein R₁ is CH₃, x′ is —CH═CH—, R₂ and R_{2′} are both H, L is absent and Ar₁ is phenyl.

8. The sulphonamide derivative according to claim 1, wherein the sulphonamide is selected from the group consisting of: 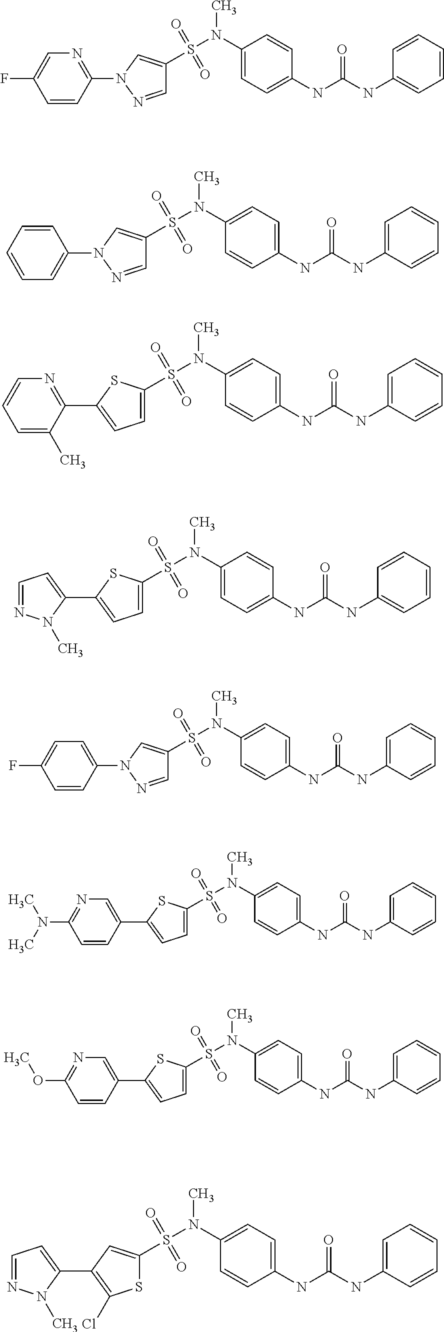 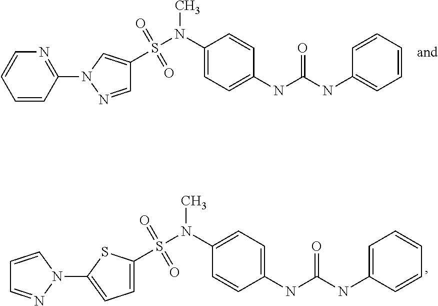
or a physiologically acceptable salt thereof.

9. The sulphonamide derivative according to claim 1, wherein the sulphonamide is 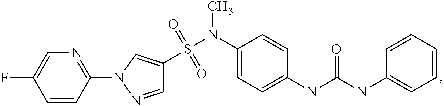
or a physiologically acceptable salt thereof.

10. A method for treating a disease that can be treated by inhibiting a collagen receptor integrin in a patient, the method comprising administering to the patient an effective amount of a sulphonamide derivative according to claim 1, or a physiologically acceptable salt thereof.

11. The method of claim 10, wherein the collagen receptor integrin is an α2β1 integrin.

12. The method of claim 10, wherein the collagen receptor integrin is an α2β1 integrin I domain.

13. (canceled)

14. A method for treating a disease selected from the group consisting of thrombosis, inflammation, cancer, vascular diseases, inflammatory bowel disease, psoriasis, arthritis, multiple sclerosis, asthma, and allergy in a patient, the method comprising administering to the patient an effective amount of a sulphonamide derivative according to claim 1, or a physiologically acceptable salt thereof.

15. A pharmaceutical composition comprising a sulphonamide derivative according to claim 1 or a physiologically acceptable salt thereof and one or more suitable adjuvants.

16. A method for preparing a sulphonamide derivative according to claim 3, comprising
reacting a compound of formula (III) 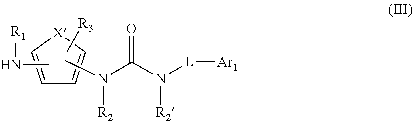
with a compound of formula (IV)
R_{B}—Ar₂-SO₂-G (IV)
where G is a leaving group;
reacting a compound of formula (V) 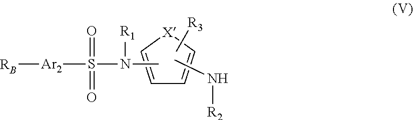
with a compound of formula (VI)
G-C(O)NR_{2′}-L-Ar₁ (VI)
where G is a leaving group; or
reacting a compound of formula (VII) 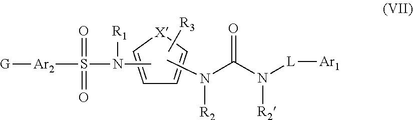
where G is a leaving group, with a compound of formula (VIII)
R_{B}-M (VIII)
where M is a leaving group such as a metal.
